(19) United States
(12) Patent Application Publication
(10) Pub. No.: US 2006/0003327 A1
(43) Pub. Date: Jan. 5, 2006

(54) Peripheral blood cell markers useful for diagnosing multiple sclerosis and methods and kits utilizing same

(76) Inventors: Anat Achiron, Tel Aviv (IL); Michael Gurevich, Rehovot (IL); Mathilda Mandel, Tel Aviv (IL); Nir Friedman, Mevaseret Zion (IL); Naftali Kaminski, Pittsburgh, PA (US)

Correspondence Address: Martin Moynihan;Anthony Castorina, Suite 207, 2001 Jefferson Davis Highway, Arlington, VA, 22202, US

(21) Appl. No.: 10/507,380

(22) PCT Filed:
(86) PCT No.: PCT/IL03/00208

Related U.S. Application Data
(60) Provisional application No. 60/365,800, filed on Mar. 21, 2002.

(51) Int. Cl. C12Q 1/68; G01N 33/53

(52) U.S. Cl. 435/6; 435/7.1

(57) ABSTRACT

Markers of multiple sclerosis and methods and kits utilizing same for diagnosing multiple sclerosis in an individual are provided.

### FIELD AND BACKGROUND OF THE INVENTION

The present invention relates generally to the field of diagnosis, treatment assessment and prognosis. More specifically, the present invention relates to peripheral blood cell expressed markers and kits and methods utilizing same for diagnosing, treating and assessing the state of multiple sclerosis (MS) in an individual. The present invention also provides cellular markers which are useful in distinguishing between different clinical courses of MS e.g.: probable, relapsing-remitting, secondary progressive or primary progressive as well as response to the therapy.

Multiple sclerosis is an autoimmune neurodegenerative disease, which is marked by inflammation within the central nervous system with lymphocyte attack against myelin produced by oligodendrocytes, plaque formation and demyelization with destruction of the myelin sheath of axons in the brain and spinal cord, leading to significant neurological disability over time. The disease frequently occurs in young adults between 20-40 years of age, is more prevalent in females than males (2:1), and has a characteristic geographical distribution—estimated prevalence in USA 120/100,000, (250,000 to 350,000 cases).

The annual cost of MS in USA was estimated about $34,000 per person, $2.2 million total lifetime cost per case or $6.8 billion yearly, in a conservative estimate of a national annual cost (Anderson D W, 1992; Whetten-Goldstain K., 1998).

Clinical Diagnosis and Evaluation of Stages of MS

Typically, at onset an otherwise healthy person presents with the acute or sub acute onset of neurological symptomatology (attack) manifested by unilateral loss of vision, vertigo, ataxia, dyscoordination, gait difficulties, sensory impairment characterized by paresthesia, dysesthesia, sensory loss, urinary disturbances until incontinence, diplopia, dysarthria or various degrees of motor weakness until paralysis. The symptoms are usually painless, remain for several days to a few weeks, and then partially or completely resolve. After a period of remission, a second attack will occur. During this period after the first attack, the patient is defined to suffer from probable MS. Probable MS patients may remain undiagnosed for years. When the second attack occurs the diagnosis of clinically definite MS (CDMS) is made (Poser criteria 1983; C. M. Poser et al., Ann. Neurol. 1983; 13, 227).

The clinical disease courses of MS are relapsing-remitting, primary or secondary progressive (Abramsky, 1997; Russell, 1998).

The relapsing-remitting course of MS (85% of patients) is characterized by acute attacks or relapses during which new neurological symptoms and signs appear, or worsen. Relapse develops within a period of several days, lasts for 6-8 weeks, than gradually resolves. During the acute relapse scattered inflammatory and demyelinating central nervous system (CNS) lesions produce varying combinations of motor, sensory, coordination, visual, and cognitive impairments, as well as symptoms of fatigue and urinary tract dysfunction. The outcome of a relapse is unpredictable in terms of neurological sequel but it is well established that with additional relapses, the probability of complete clinical remission decreases and neurological disability and handicap may develop. On average, about 60% of patients remain fully functional 10 years after the primary attack, and 25 to 30% remain fully functional 30 years after onset. Statistically, the disease does not greatly decrease life expectancy (mean decrease 12 years), although some patients become severely disabled and die from recurrent infections and complications.

Primary progressive MS (10% of patients) is characterized by slow, progressive neurological dysfunction usually in the form of a gradual myelopathy causing spasticity and ataxia. Treatment regimen varies greatly with different clinical course and severity of the disease.

The diagnosis of MS is still defined primary by clinical terms and relies on a combination of history, neurological examination and ancillary laboratory and neuro-imaging studies.

Laboratory tests for MS include: 1) CSF evaluation of IgG synthesis, oligoclonal bands; 2) MRI of the brain and spinal cord and; 3) exclusion of other autoimmune diseases by blood tests [e.g.; serum B12 level; HTLV 1 or HIV 1 titers; sedimentation rate or C-reactive protein; RA latex (Rheumatoid arthritis); ANA, anti-DNA antibodies (systemic lupus erythematosus)]. However, accurate diagnosis and prognosis in the “probable” stage, and early relapsing-remitting stages remains problematic. For example, it has been shown that positive MRI findings in the first demyelinating attack only provide a 50% successful prediction of development of clinically definite MS within 2-3 years (CHAMPS Study Group, Neurology 2002; 59:998-1005). Likewise, Villar et al (Neurology 2002; 59:877-83) found that detection of oligoclonal IgM bands with early symptoms were only partially predictive of development of clinically definite MS.

Other laboratory tests may provide some additional support for the diagnosis, but evidence of lesions disseminated in time and space remains a cardinal element of the diagnosis (Poser C M., 2001). In absence of definitive laboratory tests and pathognomonic clinical features, MS remains ultimately a diagnosis of exclusion.

Diseases that may be confused with MS are: 1) Acute disseminated encephalomyelitis (follows infections or vaccination mainly in children, fever, headaches, and meningitis common), 2) Lyme disease (antibodies to Borrelia species antigens in serum and CSF), 3) HIV associated myelopathy (HIV antibodies present), 4) HTLVI myelopathy (HTLVI antibodies present in serum/CSF), 5) Neurosyphilis (syphilis antibodies present in serum and/or CSF, 6) Progressive multifocal leukoencephalopathy (biopsy of lesions demonstrates virus by electron microscopy), 7) Systemic lupus erythematosus (CNS manifestations of lupus, antinuclear antibodies, anti-dsDNA), 8) Polyarteritis nodosa (systemic signs, micro-aneurysms demonstrated by angiographies, vasculitis demonstrated in biopsy of involved areas), 9) Sjogren's syndrome (dry eyes and mouth, antiRo and antiLa antibodies), 10) Behcet's disease (Oral/genital ulcers, antibodies to oral mucosa), 11) Sarcoidosis (CNS signs, increased protein in CSF, biopsy shows granuloma, 12) Paraneoplastic syndromes (older age group, antiYo antibodies), 13) Subacute combined degeneration of cord (peripheral neuropathy, vitamin B12 levels), 14) Sub acute myeloopticoneuropathy (adverse reaction to chlorhydroxyquinoline, mainly in Japanese), 15) Hereditary spastic paraparesis/primary lateral sclerosis (normal CSF, MRI and visual evoked potential studies), 16) Adrenomyeloneuropathy (adrenal dysfunction, neuropathy, increased plasma very long-chain fatty acids), 16) Spinocerebellar syndromes (familial, pes cavus scoliosis, abnormal reflexes, normal CSF IgG), 17) Miscellaneous—strokes, tumors, arteriovenous malformations, arachnoid cysts, Arnold-Chiari malformations, and cervical spondylosis all may lead to diagnostic dilemmas on occasion. Thus, detailed history and neurological examination must be complemented by specific laboratory tests for the correct diagnosis of MS. Clearly there is a long felt need for more powerful diagnostic tools for prediction and staging of MS.

Etiology of MS

The etiology of MS is unknown. It is suggested that a combination of genetic background and environmental factors and immune response are involved in the disease. A certain incidence of familial occurrence has been observed, with the concordance rate among monozygotic twins being 30%, a 10-fold increase over that in dizygotic twins or first-degree relatives (Steinman, 1966; Dyment et al Mol. Gen 1997; 6:1693-98). In addition, recent research indicates that the tissue damage in MS occurs as the result of pathological autoimmune responses to several myelin antigens following exposure to an as yet undefined environmental causal agent.

However, although some environmental factors have been statistically associated with the disease, none have provided correlations of any predictive value. Environmental factors seem to trigger MS in subjects who are already genetically susceptible to the illness. Most probably no one dominant gene determines genetic susceptibility, but rather many genes, each with different influence, are involved. Indeed, the initial pathogenic process could be caused by one group of genes, while others groups could be responsible for the development and progression of the disease (Oksenberg, 2001; Compston, 1997).

Microarray Analysis and MS

Microarray technology is based on hybridization of mRNA to high-density array of immobilized target sequences. Each sequence corresponds to a specific gene(s) of interest. The labeled pool of sample mRNA is subsequently hybridized to the array (chip). Application of this technology provides the capability of monitoring thousands of various genes simultaneously. Today commercial available DNA microarrays (Affymetrix, Santa Clara Calif., USA) contain elements representing 10,000, 20,000 or more genes that have been characterized in terms of function or disease association. The preparation and use of microarrays for diagnostics, research and drug development is disclosed in, inter alia, U.S. Pat. Nos. 6,324,497 and 6,468,476 to Friend et al and 6,410,229 to Lockhart et al; and Intnl Pat. Application WO 0053625C2 and A2.

Several application of microarrays in human disease have been reported, for example the identification (marker) genes involved in ovarian carcinogenesis (Ono K., 2000); classification of genes expression profiling of cutaneous malignant melanoma (Bitter M., 2000); and expression profile of Tangl-Rearing CA1 neurons in Alzheimer's disease (Stephen, 2000). Alizaden (2000) characterized gene expression in diffuse large B cell lymphoma, where two distinct gene expression patterns, characterized by different molecular forms of B cells lymphoma, were identified. In addition, microarray technology has also been applied to diagnosis and monitoring of such diverse diseases as cancer (U.S. Pat. No. 6,511,849 to Freuhauf et al), psoriasis (Intnl Pat. Application WO 20020027538 to Trepicchio et al), T-helper cell related diseases (Trepicchio et al, Intnl Pat Application WO 20020039734), Epstein-Barr disease (U.S. Pat. Nos. 6,506,553 and 6,468,476 to Smith and Parks), rheumatoid arthritis (Intnl Pat Application WO 0248310A2 to Trepicchio et al) and Reward Deficiency Syndrome, all of which are incorporated herein by reference.

In a recent review (Greenberg S A., 2001) the author discussed the potential application of DNA microarray technology for understanding neurological disorders. Using cDNA microarrays technology, brain tissue from pathology lesions and normal white matter of single MS patient were analyzed (Whitney L W., 1999). Blood genomic fingerprints were demonstrated after experimental strokes, seizures, hypoglycemia and hypoxia of rats (Yang Tang, 2001). Similarly, microarray analysis of gene expression in brainstem and spinal cord tissues from the animal models of MS (experimental autoimmune encephalomyelitis, EAE) has identified a number of differentially expressed genes from active-acute versus silent lesions (Lock C. et al Nat Med 2002; 8,500-504), and also suggested a role for the proinflammatory cytokine osteopontin in the development of EAE in mice (Chabas D et al Science 2001; 294:1731-34).

In another recent study, Ramanathan M et al (J of Immunology 2001; 116:213-19) used cDNA microarray technology to identify abnormal gene expression patterns in PBMC of relapsing-remitting MS patients. The study compared PBMC gene expression in 15 patients during remission (only) with that of 15 healthy controls, using a GeneFilters GF211 array (Research Genetics, Huntsville Ala., USA) having approximately 5200 human gene sequences. Groups of marker genes correlated with MS were disclosed, but the range of differences (fold changes) between level of gene expression in MS and control groups was only 13 to 35% for unregulated and from 11 to 43% for down regulated genes. Such small differences are probably due to the limited sensitivity of the technology employed in using GeneFilters arrays, and may not have any clinical or diagnostically mining significance. More significantly, the population of MS patients was limited, including only patients during clinical remission, who had not received any immunosupressive treatment for at least 3 months. Thus, the markers described do not provide a profile of expression patterns useful for diagnosing clinically defined MS in patients having probable MS, or for determining stages of the disease.

Trepicchio et al. (Intnl Pat. Application No. WO 02/079218 A1) also describe the use of microarray technology in determining characteristic gene expression in an animal model of MS (murine EAE) and in tissue samples from MS patients. The human samples were PBMC or brainstem tissue, collected from 60 patients manifesting a wide variety of symptoms, at different stages of MS including relapsing-remitting, primary and secondary progressive, and acute exacerbation. RNA probes prepared from these samples were hybridized to a human chip array containing approximately 14,000 gene sequences (MicroArray, Affymetrix, cat no. 510448, Santa Clara Calif.), and expression profiles compared with those of healthy controls. Determination of the panel of “MS-related” markers was based merely on fold change of greater than 2 fold (up- or downregulated), with a confidence level of p<0.01. No more stringent statistical criteria were applied. A “panel” of 300 differentially regulated genes was thus described in the PBMC samples, and another 100 in the brain lesion tissue. However, no classification of expression profiles characteristic to specific stages of the disease was provided, and the “class predictor model”, as described, using “neighborhood analysis”, was applied for attempted prediction of “MS-afflicted” or “non-diseased” samples only. Thus, the panel of markers described is not applicable to the diagnosis of stage of MS, in general, is unsuited for the prediction of clinically definite MS or probable MS patients, and is clearly non-predictive in monitoring response to treatment.

There is thus a widely recognized need for, and it would be highly advantageous to have gene expression profiles useful in distinguishing between different forms of MS e.g.: probable, relapsing-remitting, primary or secondary as well as response to the therapy, devoid of the above limitations.

### SUMMARY OF THE INVENTION

According to one aspect of the present invention there is provided a method of diagnosing a subject with multiple sclerosis, the method comprising determining a level of expression of at least one gene selected from the group consisting of the genes listed in Tables I-V in a sample obtained from the subject, wherein a substantial difference between the level of expression of the gene in the sample obtained from the subject and a normal expression level of the gene is an indication that the subject is afflicted with multiple sclerosis.

According to further features in preferred embodiments of the invention described below a method of monitoring a state of multiple sclerosis in a subject, the method comprising monitoring a level of expression of at least one gene selected from the group consisting of the genes listed in Tables I-V over a predetermined time period, wherein substantial difference between the levels of expression of the at least one gene over the predetermined time period indicates a change in a state of the multiple sclerosis in the subject.

According to further features in preferred embodiments of the invention described below monitoring the level of expression of at least one gene over the predetermined time period is effected by periodically obtaining a sample from the individual and determining the level of expression of the at least one gene in the sample.

According to still further features in the described preferred embodiments the at least one gene comprises at least 10, at least 50, at least 100, at least 250, at least 500, at least 750, at least 1000 or at least 1200 genes each independently selected from the group consisting of the genes listed in Tables I-V.

According to another aspect of the present invention there is provided a method of diagnosing a subject with multiple sclerosis, the method comprising the step of determining a level of expression of each of the genes listed in Tables I-V in a sample obtained from the subject, wherein a substantial difference between expression levels of the genes in the sample obtained from the subject and normal expression levels of the genes is an indication that the subject is afflicted with multiple sclerosis.

According to further features in preferred embodiments of the invention described below the normal expression level of the at least one gene or genes is determined by measuring the level of expression of the gene or genes in at least one control sample obtained from at least one healthy individual.

According to still further features in the described preferred embodiments the sample includes peripheral blood mononuclear cells.

According to yet further features in the described preferred embodiments the substantial difference is a difference statistically significant at a confidence level of p=0.05 as determined by at least one test selected from the group consisting of a t-test, a TNoM and an INFO score.

According to further features in preferred embodiments of the invention described below the level of expression of the at least one gene or genes is determined by quantifying a level of a protein product thereof in the sample. According to still further features in the described preferred embodiments quantifying a level of the protein is effected using a reagent which specifically binds with the protein.

According to yet further features in preferred embodiments of the invention described below the reagent comprises an antibody or fragments thereof.

According to further features in preferred embodiments of the invention described below the at least one gene or genes are selected from the genes listed in Table I.

According to still further features in preferred embodiments of the invention described below the at least one gene or genes are selected from the genes listed in Table II.

According to yet further features in preferred embodiments of the invention described below the at least one gene or genes are selected from the genes listed in Table III.

According to further features in preferred embodiments of the invention described below the at least one gene or genes are selected from the genes listed in Table IV.

According to still further features in the described preferred embodiments at least one gene or genes are selected from the genes listed in Table V.

According to yet further features in preferred embodiments of the invention described below the level of expression of the at least one gene or genes in the sample is determined by detecting the presence in the sample of a transcribed polynucleotide or portion thereof. The transcribed polynucleotide can be mRNA.

According to further features in preferred embodiments of the invention described below the transcribed polynucleotide or portion thereof is detected via a labeled probe which specifically hybridizes with the transcribed polynucleotide or portion thereof.

According to still further features in the described preferred embodiments the sample from a subject is T cells, the at least one gene or genes are selected from the genes listed in Table IV and the normal expression of the gene or genes is T-cell expression.

According to an additional aspect of the present invention there is provided a method of assessing the efficacy of a treatment regimen on multiple sclerosis in a subject, the method comprising determining a level of expression of at least one gene or genes selected from the group consisting of the genes listed in Tables I-V in samples obtained from the subject prior to, and following exposure to the treatment regimen, wherein a substantial difference in the expression level of at least one gene or genes between the samples is an indication that the treatment regimen is efficacious in treating multiple sclerosis in the subject.

According to further features in preferred embodiments of the invention described below the treatment regimen is administering at least one test compound for inhibiting multiple sclerosis.

According to still further features in the described preferred embodiments the treatment regimen is an environmental condition.

According to yet further features in the described preferred embodiments the substantial difference is a difference statistically significant at a confidence level of p=0.05 as determined by at least one test selected from the group consisting of a t-test, a TNoM and an INFO score.

According to further features in preferred embodiments of the invention described below the level of expression of the at least one gene or genes is determined by quantifying a level of a protein product thereof in the sample.

According to still further features in the described preferred embodiments quantifying a level of the protein is effected using a reagent which specifically binds with the protein.

According to yet further features in preferred embodiments of the invention described below the reagent comprises an antibody or fragments thereof.

According to further features in preferred embodiments of the invention described below the at least one gene or genes are selected from the genes listed in Table I.

According to still further features in preferred embodiments of the invention described below the at least one gene or genes are selected from the genes listed in Table II.

According to yet further features in preferred embodiments of the invention described below the at least one gene or genes are selected from the genes listed in Table III.

According to further features in preferred embodiments of the invention described below the at least one gene or genes are selected from the genes listed in Table IV.

According to still further features in the described preferred embodiments at least one gene or genes are selected from the genes listed in Table V.

According to yet further features in preferred embodiments of the invention described below the level of expression of the at least one gene or genes in the sample is determined by detecting the presence in the sample of a transcribed polynucleotide or portion thereof. The transcribed polynucleotide can be mRNA.

According to further features in preferred embodiments of the invention described below the transcribed polynucleotide or portion thereof is detected via a labeled probe which specifically hybridizes with the transcribed polynucleotide or portion thereof.

According to still further features in the described preferred embodiments the sample from a subject is T cells, the at least one gene or genes are selected from the genes listed in Table IV and the normal expression of the gene or genes is T-cell expression.

According to still further features in the described preferred embodiments the at least one gene comprises at least 10, at least 50, at least 100, at least 250, at least 500, at least 750, at least 1000 or at least 1200 genes each independently selected from the group consisting of the genes listed in Tables I-V.

According to another aspect of the present invention there is provided a kit for diagnosing multiple sclerosis in a subject, the kit comprising components suitable for determining expression levels of at least one gene selected from the group of genes listed in Tables I-V.

According to further features in the described preferred embodiments the reagents include at least one polynucleotide sequence selected capable of specifically hybridizing with an transcription product of the at least one gene and reagents for detecting and optionally quantifying a complex formed from the at least one polynucleotide sequence and said transcription product.

According to still further features in the described preferred embodiments the reagents include at least one antibody selected capable of specifically binding a polypeptide product of the at least one gene and reagents for detecting and optionally quantifying a complex formed from the at least one antibody and the polypeptide product.

According to further features in preferred embodiments of the invention described below the at least one gene is selected from the genes listed in Table I.

According to still further features in preferred embodiments of the invention described below the at least one gene is selected from the genes listed in Table II.

According to yet further features in preferred embodiments of the invention described below the at least one gene is selected from the genes listed in Table III.

According to further features in preferred embodiments of the invention described below the at least one gene is selected from the genes listed in Table IV.

According to still further features in the described preferred embodiments at least one gene is selected from the genes listed in Table V.

According to further features in preferred embodiments of the invention described below the kit further comprises packaging material identifying the kit as useful from diagnosing MS.

According to another aspect of the present invention there is provided a polynucleotide array comprising at least 10 and no more than 1500 polynucleotide sequences, wherein each of the sequences is selected capable of hybridizing with a transcription product of a polynucleotide sequence of a gene selected from the group of genes listed in Tables I-V.

According to further features in preferred embodiments of the invention described below the array is selected having polynucleotide sequences capable of diagnosing subjects suspected of suffering from multiple sclerosis. The subjects may also be suspected of suffering from probable multiple sclerosis, primary progressive multiple sclerosis, secondary progressive multiple sclerosis, and/or relapsing/remitting multiple sclerosis.

According to further features in preferred embodiments of the invention described below the gene is selected from the genes listed in Table I, II, III, IV and/or IV.

According to yet another aspect of the present invention there is provided an array comprising at least 10 and no more than 1500 antibodies or antibody fragments each capable of specifically binding a protein product of a gene selected from the group of genes listed in Tables I-V.

According to further features in preferred embodiments of the invention described below the array is selected having antibodies or antibody fragments capable of diagnosing subjects suspected of suffering from multiple sclerosis. The subjects may also be suspected of suffering from probable multiple sclerosis, primary progressive multiple sclerosis, secondary progressive multiple sclerosis, and/or relapsing/remitting multiple sclerosis.

According to further features in preferred embodiments of the invention described below the gene is selected from the genes listed in Table I, II, III, IV and/or IV.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-B are graphic representations of the differences in PMBC gene expression between MS patients and healthy subjects. RNA from Peripheral Blood Mononuclear Cells (PMBC) of 26 patients diagnosed with MS, and 18 healthy, age-matched controls was purified, labeled hybridized to a Genechip array (U95Av2, Affymetrix Inc. Santa Clara Calif., USA), scanned and analyzed according to manufacturer's recommendations. The data were normalized and fold ratios calculated for each gene of the MS samples against the geometric mean of the controls. FIG. 1A shows the number of MS specific genes detected having increased expression (fold change greater than 1.5) analyzed by t-test (red line), TNoM (green line) and INFO (blue line), compared with random occurrence (black line), at confidence levels (False Discovery Rates, FDR) of 90% (p=0.10) to 100% (p=0). Note the high level of significant MS-related gene expression at 95% FDR and above (arrows) (1249 distinguished genes). FIG. 1B is an infogram of the 1249 genes most significantly (p<0.05 on all three tests) distinguishing MS patients (MS) from (control) healthy controls, determined as above. Each spot represents expression of a specific gene; color intensity of overexpressed (green) and under-expressed (red) genes indicates fold increase as compared to controls. Gray color indicates genes showing no difference in expression between MS and controls.

FIGS. 2A-B are graphic representations of the differences in PMBC gene expression between MS patients during acute relapse, and MS patients in remission. RNA from PMBC of 12 relapsed, and 14 clinically in remission patients was purified, labeled, hybridized and analyzed as described for FIGS. 1A-B hereinabove. FIG. 2A shows the number of acute relapse-specific genes detected having increased expression in relapse, as analyzed by t-test (red line), TNoM (green line) and INFO (blue line), compared with random occurrence (black line), at confidence levels (False Discovery Rates, FDR) of 90% (p=0.10) to 100% (p=0). 735 genes were detected having significant relapsing-related gene expression at 95% FDR and above. FIG. 2B is an infogram analysis of the 735 genes most significantly (p<0.05 on all three tests) distinguishing acute relapsing MS patients (Relapse) from MS patients in remission (Remission). Note the different profiles of gene expression in patients undergoing treatment (Relapse+ and Remission+) compared with untreated patients (Relapse− and Remission−).

FIG. 3 is a pie chart diagram showing the breakdown, by functional character, of specific genes displaying up- or down-regulation in MS-derived MOG-reactive T-cell lines, as compared to normal-derived MOG-reactive T-cell lines. Significant MOG reactive MS-related genes are defined as genes with TNOM=0 and p=0.057 as compared to normal MOG-reactive T-cells.

FIG. 4 is a graphic representation of the differences in gene expression between MOG-stimulated T-cell lines from MS patients and healthy controls. RNA from MOG-stimulated T-cells of 4 MS patients and 3 matched controls was purified, labeled, hybridized and analyzed as described for FIGS. 1A-B hereinabove. Panel A shows a cluster analysis of 150 differentially expressed genes analyzed as described hereinabove (TNoM=0, p<0.05) distinguishing T-cells of MS (MS) patients from controls (Controls). Panel B shows a cluster analysis of the 43 most informative genes (TNoM=0, p<0.05, and fold change>1.5). Each row represents a gene, and each column represents a T-cell line form a different subject. Yellow color indicates genes with an increased expression relative to controls are yellow, and blue color indicates genes with relative decreased expression.

### DESCRIPTION OF THE PREFERRED EMBODIMENTS

The present invention is of methods and kits for diagnosing multiple sclerosis in subjects, using novel gene expression profiles derived from peripheral blood cells. Specifically, the present invention can be used to diagnose MS in early stages of the disease, to determine clinical stage and predict the course of the disease in patients with a unclear diagnoses, to provide definition and prognostic information in patients with probable MS, to assess and monitor MS therapies and to screen new and established drugs and treatments for MS.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the Examples and drawings. The invention is capable of other embodiments or of being practiced or carried out in various ways. Also, it is to be understood that the phraseology and terminology employed herein is for the purpose of description and should not be regarded as limiting.

The present invention provides previously unavailable accuracy in predicting and staging MS, by identifying genes and groups of genes specifically over- and under-expressed in PBMC of patients at various stages of their disease.

As is further described in the Examples section which follows, the present inventors have conducted a broad scale analysis of PMBC expressed genes using hybridization of biotin-labeled PBMC mRNA to more than 12,000 human gene sequences provided on DNA chips. By utilizing specialized statistical analysis approaches, the present inventors identified in the microarray data the most highly informative expression profiles.

As mentioned hereinabove, multiple sclerosis is a chronic, multi-factorial neurodegenerative disease of unknown etiology, the diagnosis and classification of which remains largely clinical in nature. Identification of the stages and progression of the disease, particularly definition of the probable MS stage, is crucial to determination of optimal treatment regimen and development of effective therapies. However, the complexities of autoimmune interactions, and the variability of MS in different individuals have made diagnosis and subsequent prognosis using traditional methods inexact and challenging. Methods for more accurate diagnosis of MS are greatly needed.

The profiles of MS-related genetic markers listed in Table I represent genes exhibiting differential expression in PBMCs from a large sample of MS patients, compared to that of age-matched healthy controls. Abundance of specific gene transcripts, represented by the intensity of label hybridizing to individual sequence loci of the MicroArray (Affymetrix Inc, Santa Clara Calif.), was recorded and quantified according to the manufacturers recommended protocols (such as GeneChip 3.0 software from Affymetrix). However, rather than composing the profile of differentially expressed genes based on probabilities using simple distribution of mean intensities, as has been reported by Ramanathan et al (J Immunol 2001; 116:213-219), informative genes were selected based on the degree to which they were predictive of classification of the sample as “diseased” or “not diseased”. By applying the rigorous three-pronged statistical analysis described in detail hereinbelow, 1249 genes most informative in distinguishing between diseased and otherwise not diseased patients were identified (see Table I). By applying an even more restrictive analysis of the data in Table I (see Table II, Bonfferoni analysis), a subset of the 300 highest scoring genes was identified. These MS marker genes comprise both over-expressed and downregulated genes, and represent of a diverse group of functional gene categories. Additional analysis of the markers uncovered herein also led to the identification of another restricted marker set which can be accurately utilized to diagnose probable MS patients. As is further described hereinbelow, the identification of such a marker set represents a significant breakthrough since it enables to treat individuals at a much earlier stage of MS then previously possible.

Thus, according to one aspect of the present invention there is provided a method of diagnosing a subject with multiple sclerosis by determining a level of expression of at least one gene of the genes listed in Tables I-V in a sample obtained from the subject, wherein a substantial difference between the level of expression of the gene in the sample obtained from the subject and a normal expression level of the gene is an indication that the subject is afflicted with multiple sclerosis.

Normal expression levels of a marker or markers are obtained from isolated or cultured PMBCs (e.g., T-cell cultures), or samples obtained from individuals not affected with MS. A substantial difference is preferably of a magnitude that is statistically significant (see the Examples section for more detail). In particularly preferred embodiments, the marker is increased or decreased relative to control samples by at least 2-, 3-, 4-, 5-, 6-, 7-, 8-, 9-, or 10-fold or more. Similarly, one skilled in the art will be well aware of the fact that a preferred detection methodology is one in which the resulting detection values are above the minimum detection limit of the methodology utilized.

As is further described in the Examples section which follows, the marker listed in Tables I-V were identified in peripheral blood cells. As such, the sample obtained from the individual is preferably a peripheral blood sample or any sample which includes blood cells such as T-cells. In a preferred embodiment, the sample is blood, thymus, spleen, lymph, pus, or bone marrow. However, it will be apparent to one skilled in the art that PMBCs may be present as an infiltrate in many other tissues, and that such tissues may also serve as samples in which the presence, activity, and/or quantity of the markers of the invention may be assessed. The tissue samples containing one or more of the markers themselves may be useful in the methods of the invention, and one skilled in the art will be well aware of methods by which such samples may be conveniently obtained, stored, preserved and processed. For further description relating to collection and processing of blood samples please see the Examples section which follows.

As is detailed in the Examples section below, analysis of PBMC genes differentially expressed in MS, according to the methods described herein, revealed groups of genes of specific interest in MS. Genes that are most significantly over expressed, or downregulated in MS can indicate members of pathways important to disease development or pathology. Strongly overexpressed genes, according to Tables I and II, include SLAM (signaling lymphocyte activation molecule, GenBank Accession No. U33017), LEF1 (lymphoid enhancer-binding factor 1, GenBank Accession No. AL099409), LRP5 (low density lipoprotein receptor-related protein 5, GenBank Accession No. AF077820), LILRB (leukocyte immunoglobulin-like receptor, GenBank Accession No. AF004230), LY75 (lymphocyte antigen 75, GenBank Accession No. AF011333), CDW52 (GenBank Accession No. N90866), PIP5K1-gamma (Phosphatidylinositol-4-phosphate 5-kinase, type 1, gamma, GenBank Accession No. AB011161), MAP4 (Microtubule-associated protein 4, GenBank Accession No. M64571), CTSK (Cathepsin K, GenBank Accession No. X82153) and CTSB (Cathepsin B, GenBank Accession No. L22507). Strongly down-regulated genes include IL1B (Interleukin 1 beta, GenBank Accession No. M15330), TRAF6 (GenBank Accession No. U78798), SCYA20 (GenBank Accession No. U64197), IL1R (type1 receptor, GenBank Accession No. M27492), IL1RAP (receptor accessory protein, GenBank Accession No. AB006537) and IL1RN (receptor antagonist, GenBank Accession No. X52015), TGFB1 (Transforming growth Factor beta. 1, GenBank Accession No. X05839), SKI (v-ski sarcoma viral oncogene homologue, GenBank Accession No. X15218), VEGF (Vascular endothelial growth factor, GenBank Accession No. M63978), IGFBP4 (Insulin-like growth factor binding protein 4, GenBank Accession No. U20982), EREG (epiregulin, GenBank Accession No. NM_{—}001432.1), and NR4A1, NR4A2, NR4A3 (nuclear receptor family genes, GenBank Accession Nos. NM_{—}002135.1, X75918 and U12767, respectively).

Functional groups of genes strongly represented in the profile of most significantly differentially regulated genes in MS include, inter alia, apoptosis-related genes, T-cell activation and expansion related genes, cell proliferation related genes and epidermal growth factor genes. Many of the marker genes identified are associated with other MS-related genes, according to Tables I-V.

It will be appreciated that although a single marker can be used for diagnosis, diagnostic accuracy typically increases with an increase in the number of markers utilized.

As such, the diagnostic method of the present invention preferably utilizes a marker set that can range anywhere from 2 genes to 1200 genes. For example, the present method can utilize at least 10, at least 50, at least 100, at least 250, at least 500, at least 750, at least 1000 or at least 1200 genes each independently selected from the group consisting of the genes listed in Tables I-V. Most preferably the markers utilized are selected from the sequences listed in Table II.

The markers sets utilized can be selected according to a statistical significance or fold change thereof (provided for each marker in Tables I-V), a higher significance and higher fold change indicating higher probability of marker accuracy. For example, a selected marker set can encompass markers displaying a high statistical significance (low P-value), preferably a P-value lower than 5.0E-02, more preferably lower than 5.0E-04, most preferably, lower than 5.0E-06. Alternatively, markers can be selected according to shared features of the marker gene. For example, gene markers of similar cellular function (e.g., genes of a signaling pathway such as apoptosis) or markers displaying similar activity (e.g., enzymes of the same enzyme family) can be grouped into specific marker sets.

Each marker set may be considered individually, although it is within the scope of the invention to provide combinations of two or more marker sets for use in the methods and compositions of the invention to increase the confidence of the analysis.

As used herein, the terms “polynucleotide” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

As used herein, a “gene” includes a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art, some of which are described herein. A “gene product” includes an amino acid (e.g., peptide or polypeptide) generated when a gene is transcribed and translated.

As used herein, a “probe” is defined as an oligonucleotide that is provided as a reagent to detect a target present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

As used herein, “expression” includes the process by which polynucleotides are transcribed into mRNA and translated into peptides, polypeptides, or proteins. “Differentially expressed”, as applied to a gene, includes the differential production of mRNA transcribed from a gene or a protein product encoded by the gene. A differentially expressed gene may be overexpressed or underexpressed as compared to the expression level of a normal or control cell. In one aspect, it includes a differential that is 2.5 times, preferably 5 times or preferably 10 times higher or lower than the expression level detected in a control sample. The term “differentially expressed” also includes nucleotide sequences in a cell or tissue which are expressed where silent in a control cell or not expressed where expressed in a control cell.

As used herein, the term “polypeptide” is defined as a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

As used herein, the term “marker” is defined as a polynucleotide or polypeptide molecule which is present or absent, or increased or decreased in quantity or activity in subjects afflicted with multiple sclerosis, or in cells involved in multiple sclerosis. The relative change in quantity or activity of the marker is correlated with the incidence or risk of incidence of multiple sclerosis or progression from one stage of the disease to another.

Although all of the markers listed in Tables I-V can be used in diagnosis of MS, an additional object of the present invention was to identify those markers which can be utilized to diagnose specific clinical forms and/or stages of MS.

Accurate clinical tools for specific diagnosis of disease stages in MS are presently unavailable.

As a result of comprehensive studies conducted in efforts to evaluate specific gene expression in relation to clinical disease phases, the present invention provides, for the first time, specific markers sets which can be utilized in accurate diagnosis of specific forms and stages of MS

As is illustrated in Example II of the Examples section which follows, the present invention provides marker sets which can be accurately utilized to diagnose acute relapse, remission and probable stages of MS (Tables III-V).

Of particular importance is the marker set provided in Table V. As is described in the Examples section which follows, the present inventors also uncovered cellular markers which distinct between disease-related and non-disease related T-cell myelin reactivity. Although MS appears to be caused by autoimmune T-cells activated against myelin self-antigens, myelin-reactive T-cells have been demonstrated in healthy subjects as well. Thus, distinction between disease-related and non-disease related T-cell myelin reactivity is of great clinical and investigational importance.

Cellular markers which distinct between disease-related and non-disease related T-cell myelin reactivity include down-regulating apoptosis associated genes, up regulating anti-apoptotic genes and genes responsible for increased expansion capability of autoreactive T cells and enhanced ability to penetrate the CNS. Thus, the markers of Table V include genes involved in perpetuating pathologic cellular proliferation and tissue destruction within the CNS characteristic of MS, along with increased resistance to regulation. This marker set accurately defines the requirements for an individual to develop MS, and thus has important predictive value, especially in diagnosing individuals having MS in the “probable” stage.

The identification of these markers significantly advances the field of MS diagnosis and treatment as well as provides tools which will enable elucidation of the mechanisms underlying MS formation and progression, ultimately leading to formulation of efficient, stage specific, treatment regimens.

The markers of the invention may be nucleic acid molecules (e.g., DNA, cDNA, or RNA) or the polypeptides encoded thereby. As such, detection of markers in a sample obtained from an individual can be effected using various detection methods well known to the ordinary skilled artisan.

Briefly, measurement of the relative amount of nucleic acid or polypeptide molecules can be effected by any method known in the art (see, e.g., Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989; and Current Protocols in Molecular Biology, eds. Ausubel et al. John Wiley & Sons: 1992). Typical methodologies for RNA detection include RNA extraction from a cell or tissue sample, followed by hybridization of a labeled probe (e.g., a complementary nucleic acid molecule) specific for the target RNA to the extracted RNA, and detection of the probe (e.g., Northern blotting). Typical methodologies for polypeptide detection include activity assays in cases of known enzymes, protein extraction from a cell or tissue sample, followed by hybridization of a labeled probe (e.g., an antibody) specific for the target protein to the protein sample, and detection of the probe. The label group can be a radioisotope, a fluorescent compound, an enzyme, or an enzyme co-factor. Detection of specific polypeptide and nucleic acid molecules may also be assessed by gel electrophoresis, column chromatography, direct sequencing, or quantitative PCR (in the case of nucleic acid molecules) among many other techniques well known to those skilled in the art.

Probes based on the nucleotide sequence of a marker gene or of a nucleic acid molecule encoding a marker polypeptide of the invention can be used to detect transcripts or genomic sequences corresponding to the marker gene(s) and/or marker polypeptide(s) of the invention. In preferred embodiments, the probe comprises a label group attached thereto, e.g., the label group can be a radioisotope, a fluorescent compound, an enzyme, or an enzyme co-factor. Such probes can be used as a part of a diagnostic test kit for identifying cells or tissue which misexpress (e.g., over- or under-express) a marker polypeptide of the invention, or which have greater or fewer copies of a marker gene of the invention. For example, a level of a marker polypeptide-encoding nucleic acid in a sample of cells from a subject may be detected, the amount of mRNA transcript of a gene encoding a marker polypeptide may be determined, or the presence of mutations or deletions of a marker gene of the invention may be assessed. The invention further encompasses nucleic acid molecules that differ from the nucleic acid sequences of the genes set forth in Tables I-V, due to degeneracy of the genetic code and which thus encode the same proteins as those encoded by the genes shown in Tables I-V.

An isolated marker protein, or a portion or fragment thereof, can be used as an immunogen to generate antibodies that bind marker proteins using standard techniques for polyclonal and monoclonal antibody preparation. A full-length marker protein can be used or, alternatively, the invention provides antigenic peptide fragments of these proteins for use as immunogens. The antigenic peptide of a marker protein comprises at least 8 amino acid residues of an amino acid sequence encoded by a gene set forth in Tables I-V, and encompasses an epitope of a marker protein such that an antibody raised against the peptide forms a specific immune complex with the marker protein. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of the marker protein that are located on the surface of the protein, e.g., hydrophilic regions, as well as regions with high antigenicity.

An anti-marker protein antibody (e.g., monoclonal antibody) can be used to isolate a marker protein of the invention by standard techniques, such as affinity chromatography or immunoprecipitation. An anti-marker protein antibody can facilitate the purification of natural marker proteins from cells and of recombinantly produced marker proteins expressed in host cells. Moreover, an anti-marker protein antibody can be used to detect marker protein (e.g., in a cellular lysate or cell supernatant) in order to evaluate the abundance and pattern of expression of the marker protein. Anti-marker protein antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (J. Mol. Biol. 1990; 215:403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to marker protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

It will be appreciated that non-coding sequences, such as promoter or other regulatory sequences of marker genes may be used as probes in the context of the present invention. Thus, the expression of groups of functionally related genes, responsive to similar signals important to the pathogenesis or progression of multiple sclerosis, may be assessed.

It will be appreciated that in certain cases the genes themselves can serve as markers. For example, mutations in the nucleic acid sequence of a gene (e.g., non-sense, mis-sense deletion and the like) which result in lower expression levels of the gene or lower activity of the gene product may be correlated with MS. Similarly, a duplication of the gene, which can result in higher expression levels or mutations which result in higher activity can also be correlated with MS.

Detection of the presence or number of copies of all or a part of a marker gene of the invention may be performed using any method known in the art. Typically, it is convenient to assess the presence, quantity and quality of genomic DNA by Southern analysis, in which total DNA from a cell or tissue sample is extracted, is hybridized with a labeled probe (e.g., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

In cases where detection involves discrete marker sets, the detection method of the present invention preferably employs marker probes which are conjugated to a solid support. For example, polynucleotide probes capable of specifically hybridizing with polynucleotide markers of the present invention (e.g., mRNA) may be coupled to an array (e.g., a GeneChip array for hybridization analysis), to a resin (e.g., a resin which can be packed into a column for column chromatography), or a matrix (e.g., a nitrocellulose matrix for northern blot analysis). The immobilization of molecules complementary to the marker(s), either covalently or noncovalently, permits a discrete analysis of the presence or activity of each marker in a sample. In an array, for example, polynucleotides complementary to each member of a marker set may individually be attached to different, known locations on the array (region-specific arrays). The array may be hybridized with, for example, polynucleotides extracted from a blood sample obtained from a subject. The hybridization of polynucleotides extracted from the sample with the array at any location on the array can be detected, and thus the presence or quantity of the marker in the sample can be ascertained. In a preferred embodiment, a “GeneChip” array is employed (e.g., an Affymetrix type array). Similarly, Western analyses may be performed on immobilized antibodies specific for different polypeptide markers hybridized to a protein sample from a subject.

It will also be apparent to one skilled in the art that the probes of the array need not bind with the entire marker molecule. A probe designed to bind a portion of the marker of sufficient length for detection purposes (e.g., for hybridization), for example, a portion of the marker which is 7, 10, 15, 20, 25, 30, 35, 40, 45, 50, 55, 60, 65, 70, 75, 100 or more nucleotides or amino acids in length may be sufficient for detection purposes.

Polynucleotide probes can be synthesized using any known synthesis method. Preferably, synthesis is effected using on-chip lithography methodology in a manner similar to that utilized for the synthesis of Affymetrix chips (www.affymetrix.com). Additional methods of array production and methodology are described in detail in the U.S. patent applications cited in the Background section hereinabove.

Antibody probes useful for detecting polypeptide markers can be generated using various well known techniques. For example, monoclonal antibodies which can be used per se or as a basis for antibody fragments (scFv, Fab etc) can be synthesized using isolated Hybridomas. In such an approach, a protein corresponding to a marker of the invention is isolated (e.g., by purification from a cell in which it is expressed or by transcription and translation of a nucleic acid encoding the protein in vivo or in vitro using known methods. A vertebrate, preferably a mammal such as a mouse, rat, rabbit, or sheep, is immunized using the isolated protein or protein fragment. The vertebrate may optionally (and preferably) be immunized at least one additional time with the isolated protein or protein fragment, so that the vertebrate exhibits a robust immune response to the protein or protein fragment. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein or protein fragment.

The invention also includes an array comprising a marker(s) of the present invention. The array can be used to assay expression of one or more genes in the array.

In one embodiment, the array can be used to assay gene expression in a tissue of multiple sclerosis patients at different stages of the disease to ascertain stage specificity of genes in the array. In this manner, more than about 30,000 genes can be simultaneously assayed for expression. This allows a profile to be developed showing a battery of genes specifically expressed in one or more stages of the disease.

In addition to such qualitative determination, the invention allows the quantitation of gene expression. Thus, not only stage specificity, but also the level of expression of a battery of stage specific genes is ascertainable. Thus, genes can be grouped on the basis of their expression per se, and level of expression in that stage of the disease.

The detection arrays described herein are preferably packaged in kits identified for use in detecting MS in general or for detecting specific stages of MS. The kit can further include reagents suitable for the detection of polynucleotide hybridization or antibody binding and instructions for effecting diagnosis using the kit components and suitable detection hardware (e.g., detection microscope) and software (e.g., detection and analysis software). For further description of such hardware and software and detection reagents please see www.affymetrix.com.

Thus, the present invention provides methods useful for diagnosing MS including specific stages or states of the disease and also a risk of developing the disease.

These methods involve isolating a sample from a subject (e.g., a sample containing T-cells), detecting the presence, quantity, and/or activity of one or more markers of the invention in the sample relative to a normal sample. Observing a significant increase or decrease in one or more markers in the test sample indicates the presence or risk of presence of MS.

Using specific marker sets, the present invention also provides methods of assessing the severity or stage of MS in a subject.

As detailed hereinabove, a major concern in treatment of multiple sclerosis is accurate early diagnosis following the first acute attack. At present, clinical studies indicate that only 40-50% of individuals suffering a first acute attack will progress to clinically definite MS. Thus, treatment protocols most commonly suspend treatment of these patients defined as probable MS, until the appearance of a second attack, which may entail years of waiting and uncertainty. It will be appreciated that early and accurate detection of the portion of probable MS patients likely to progress to further stages of the disease can save undue suffering and expense, and, more importantly, provide early treatment and a better prognosis for the portion of probable MS patients likely to progress to more severe stages. The present invention provides, for the first time, marker genes for probable MS, as well as for relapsing vs. remitting MS.

The present invention also provides methodology which can be used to assess the efficacy of an MS treatment regimen and/or the effect of environmental factors or diet on the progression of MS.

These methods involve isolating a sample from a subject (e.g., a sample containing T-cells) suffering from MS who is undergoing treatment which includes drug therapy, exposure to a predetermined environmental condition and/or a specific diet, detecting the presence, quantity, and/or activity of one or more markers of the invention in test samples obtained from the subject prior to and following treatment or in a test sample obtained from the subject relative to a sample obtained from an individual suffering from MS who is not undergoing any treatment and/or relative to a sample obtained from an individual not suffering from MS and undergoing treatment. The levels of markers in the samples are compared, and significant increases or decreases in one or more markers in the test sample following treatment relative to the other samples are observed, and correlated with the severity or stage of MS. By assessing whether MS has been lessened or alleviated, the ability of the treatment or therapy to treat MS is also determined.

It will be appreciated that the present invention also provides methods of treating (e.g., inhibiting) the formation or progression of MS. These methods involve isolating a sample from a subject (e.g., a sample containing PMBCs such as T-cells), detecting the presence, quantity, and/or activity of one or more markers of the invention in the sample relative to a normal sample and observing significant increases or decreases in one or more markers in the test sample. For markers that are significantly decreased in expression or activity, the subject may be administered that expressed marker protein, or may be treated by the introduction of mRNA or DNA corresponding to the decreased marker (e.g., by gene therapy), to thereby increase the levels of the marker protein in the subject. For markers that are significantly increased in expression or activity, the subject may be administered mRNA or DNA antisense to the increased marker (e.g., by gene therapy), or may be administered antibodies specific for the marker protein, to thereby decrease the levels of the marker protein in the subject. In this manner, the subject may be treated for MS or MS related condition.

In another embodiment, the methods further involve obtaining a control biological sample (e.g., nondiseased tissue) from a control subject, contacting the control sample with a compound or agent capable of detecting marker protein, mRNA, or genomic DNA, such that the presence of marker protein, mRNA or genomic DNA is detected in the biological sample, and comparing the presence of marker protein, mRNA or genomic DNA in the control sample with the presence of marker protein, mRNA or genomic DNA in the test sample.

The invention also provides methods for identifying modulators, i.e., candidate or test compounds or agents (e.g., peptides, peptidomimetics, peptoids, small molecules or other drugs) which (a) bind to the marker, or (b) have a modulatory (e.g., stimulatory or inhibitory) effect on the activity of the marker or, more specifically, (c) have a modulatory effect on the interactions of the marker with one or more of its natural substrates (e.g., peptide, protein, hormone, co-factor, or nucleic acid), or (d) have a modulatory effect on the expression of the marker. Such assays typically comprise a reaction between the marker and one or more assay components. The other components may be either the test compound itself, or a combination of test compound and a natural binding partner of the marker. The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; (see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Subjects—Blood was obtained from patients or controls after written informed consent. For comparison of healthy controls and MS patients, and between MS patients in acute relapse or remission: Gene expression profiles of 26 patients (20 females, mean age 41.0±2.5 years) with definite diagnosis of MS according to Poser criteria (8), a relapsing-remitting disease course, and brain magnetic resonance imaging ascertaining the diagnosis (9) were compared with eighteen (18) age-matched healthy subjects (16 females). For comparison of transcriptional profiles in MOG-reactive T-cells: Four MS female patients (mean age 38±4.2 years, mean disease duration 9.3±3.3 years) having a definite MS according to Poser criteria (10), a relapsing-remitting disease course, neurological disability evaluated by the expanded disability status scale (EDSS, 11) between 2 to 5.0, and brain MRI supporting the diagnosis of MS, and three age- and sex-matched healthy controls were included in the study. None of the patients received immunomodulatory drugs or steroid treatment for at least three months prior to when blood was drawn. The studies were approved by the institutional review board and the Israel Ministry of Health.

mRNA preparation—Total RNA was isolated from Ficoll™ isolated Peripheral Blood. Mononuclear Cells (PBMC) or from MOG-stimulated T cell lines (2×10⁷ cells) by ice-cold TRIZOL Reagent (Gibco, BRL). Poly-A mRNA was isolated using a mini-kit (Oligotex, Qiagen) and used as a template for double-stranded cDNA synthesis using oligo (dT)-24 primers containing a T7 RNA polymerase promoter site added to the 3′-end (Genset). After phenol/chloroform extraction cDNA was used as a template for in vitro transcription (Ambion T7 Megascript system) with biotin labeled nucleotides (Enzo Diagnostics). Labeled cRNA was fragmented, quantified by spectrophotometer, and hybridized to the microarrays.

Microarray gene analysis—Each Genechip (U95Av2) which carries probes for 12,625 (or U133A with 22,000 for patients with probable MS diagnosis) transcripts was hybridized with 10 μg/200 μl hybridization mix, stained and scanned (Hewlett Packard, GeneArray™ scanner G2500A) according to manufacturer protocol (Affymetrix Inc, Santa Clara, Calif.). Scaling procedure was performed to an average intensity of 600 per gene. A value of 20 was assigned to all measurements lower then 20. For comparison of healthy controls and MS patients, and between MS patients in acute relapse or remission: All data was normalized by dChip software and fold ratios were calculated for each gene of the samples against geometric means of the matched controls. For comparison of transcriptional profiles in MOG-reactive T-cells: Genes that did not have at least one average difference intensity value≧100 or were present at least once by Affymetrix criteria, were not included in the analysis.

Data analysis—The analysis was performed according to the analytical approach as previously described (24-26). Genechip 4 software (Affymetrix Inc, Santa Clara, Calif.) was used for analysis of the scanned arrays. Fold ratios were calculated for each gene of the samples against the geometric mean of matched controls. For comparison of transcriptional profiles in MOG-reactive T-cells: To determine the most informative genes threshold number of misclassifications (TNoM) score was applied. This score counts the number of classification errors that occur between compared groups for each gene of the dataset. The best threshold (TNoM=0) implies that no errors have been counted and the distinction between the two groups in relation to the expression level of a specific gene is maximal. To select a group of strongly differential expression, t-test p-value (comparing expression levels of genes from MS patients vs. healthy controls) were also computed. Genes with TNoM=0, fold-change>1.5 (either up or down regulated) and corresponded t-test P value<0.05, were designated as most informative. For comparison of healthy controls and MS patients, and between MS patients in acute relapse or remission: The data was analyzed by the classic parametric t-test, and the following non-parametric tests: (i) Threshold number of misclassifications (TNoM) method and (ii) INFO score that measures the misclassifications made by a simple threshold in terms of the information lost. Analysis was performed between MS patients and the control group for each gene of the dataset as well as between subgroups of patients. Only informative MS related genes (p<0.05 in all three statistical tests) were included. To retrieve the most informative genes, the False Discovery Rate (FDR) method (14) that ranks and tests all “P” values against different thresholds was used. The degree of significance by the Bonferroni threshold method, which evaluates the allowed error probability divided by the number of genes measured, and ensures that each and every validated scoring event is indeed a significant event, was also calculated.

Validation Strategy—To further assess the predictive power of the data sets, computerized analysis by the Leave-One-Out-Cross-Validation (LOOCV) statistical method was performed. The method simulates removal of a single sample every trial and trains on the rest. The procedure is repeated until each sample is left out once and the number of correct and incorrect predictions is counted.

### Example I

### Accurate Gene Expression Profiles of MS

In order to provide an accurate, reliable profile of gene markers for diagnosis and evaluation of MS, DNA chip analysis was used to compare multiple gene expression patterns of PBMCs from patients with different clinical forms of MS. After informed consent blood was obtained from 26 patients (20 females, mean age 41.0±2.5 years) with definite diagnosis of MS according to Poser criteria, a relapsing-remitting disease course, and brain magnetic resonance imaging ascertaining the diagnosis. Eighteen age-matched healthy subjects (16 females) served as controls. PBMC gene expression of 12,625 human genes was analyzed as described hereinabove, using Ficoll™ for preparation of PBMCs and total RNA purification and sample preparation according to the instructions of Affymetrix, Inc (Affymetrix, Santa Clara Calif., USA). In order to determine the most informative genes, unique computerized scoring methods, as yet not applied to analysis of data regarding MS, were employed. In brief, a gene is designated as informative based on the degree to which its tissue expression level is predictive of an independent classification of the tissue sample as “diseased” or “not diseased”, as previously described by Ben-Dor et al (J Comput Biol 2000; 7:559-63) and applied to the analysis of breast cancer and melanoma using cDNA arrays (for review see Freidman N et al Ernst Schering Res Found Wkshp 2002; 38:109-31). The scores used in this study were:

TNoM (Total Number of Misclassifications)—the number of classification errors committed when using the best simple threshold to distinguish between two classes (diseased or not diseased) based on the expression levels of a specific gene.

INFO—an estimate of the uncertainty remaining about accuracy of a sample classification (diseased or not diseased) after the incorporation of predictions based on expression of an individual gene is given (a lower “INFO” score indicates a higher predictive value for a given gene).

Gaussian (t-test)—The overlap between distributions of expression levels for genes in two classes. The score is based on normality assumptions.

One of the advantages of the analytic methods used here is their amenability to rigorous statistical benchmarking. Using this unique analysis, the number of informative genes per score expected in a random classification can be calculated, and then this estimated number of high scoring (or informative) genes can be compared to the actual number of informative genes (per score) measured in a dataset.

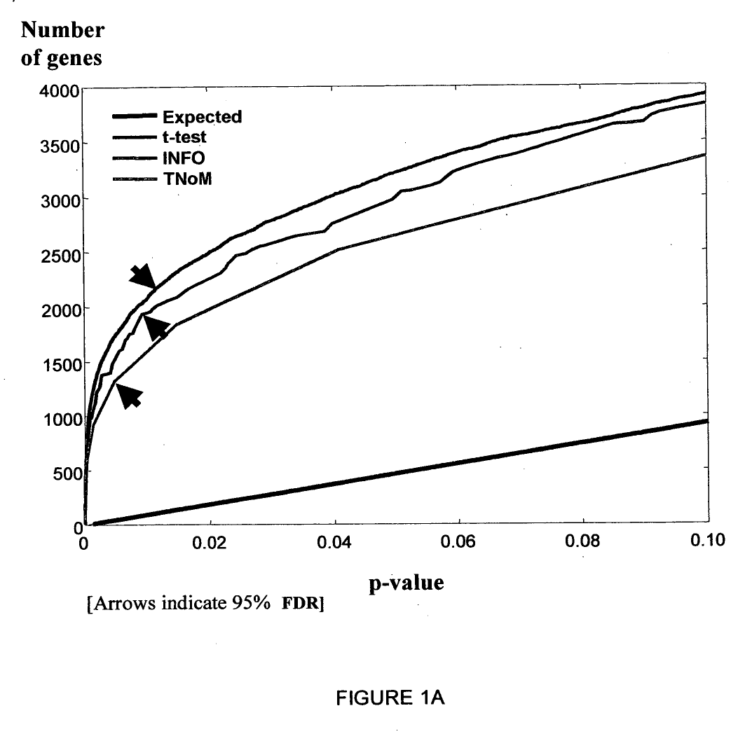

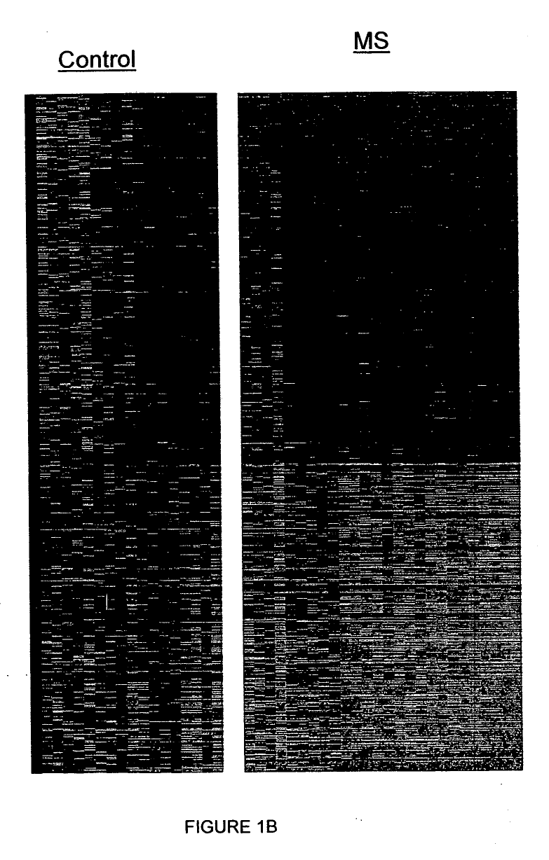

Comparison of the gene expression profiles shows that gene expression of PBMC in MS patients is significantly different from that in healthy subjects. Under the null-hypotheses that the separation of the samples is random despite genetic heterogeneity between tested groups, observed significant overabundance of informative genes was observed (FIG. 1A). The difference between expected and observed number of genes with significant p value in all 3 statistical tests (t-test, TNoM, INFO) performed, indicates that the diversity in gene expression observed in PBMC is biologically significant.

The predictive power of the data sets results was assessed by performing computerized error estimates based on leave-one-out cross validation (LOOCV) trials. The results disclosed only 3 classification errors. This low rate of error estimates suggest that the gene expression signature in MS is reliable for the diagnosis of the disease using peripheral blood and confirms that the patterns we observed accurately represent significant biologic phenomena associated with MS. The false discovery rate (FDR) method distinguished 1249 most informative genes that pass 95% FDR on all three statistical tests (t-test, TNoM, INFO) at p<0.05 (FIG. 1B and Table I).

Confirmation of gene microarray expression findings was performed by RT-PCR for the following five randomly selected genes: EGFL5, P44, GS3686, MX1 and CCR2. Significant correlations (coefficients ranged from 0.76 to 0.98) were found between the relative number of expression genes analysis and the RT-PCR profile. The data from microarray hybridizations was further tested against the strict Bonferroni threshold method from all three statistical tests, as described hereinabove, resulting in 300 top scoring genes that distinguish between MS and healthy subjects. (Table II).

The 1249 most informative genes (681 up-regulated, 569 down-regulated, Table I) consist of inflammatory, apoptosis and cell signaling pathways components, cytokines, antigen presentation molecules and chemokines as well as number of expressed sequence tags (ESTs).

Over-expressed genes in MS—The most abundant over-expressed transcripts unique to MS include: (i) SLAM (signaling lymphocyte activation molecule) a member of the immunoglobulin gene superfamily that is involved in T-cell stimulation. SLAM potentiates T-cell expansion and was described as CD28 independent co-stimulatory molecule, selectively increasing interferon gamma production and dysregulating type 1 and type 2 cytokine production in MS upon T-cell receptor activation. The surprising observation of SLAM upregulation suggests an enhanced proliferation of autoreactive T cells in MS patients; (ii) LEF1 (lymphoid enhancer-binding factor 1) one of the transcriptional factors expressed in pre-B and T cells, and known to be associated with T cell receptor (TCR) stimulation and apoptosis survival of pro-B cells (19); (iii) LRP5 (low density lipoprotein receptor-related protein 5) a of cell receptor protein required for LEF1 activation; (iv) LILRB (leukocyte immunoglobulin-like receptor), a protein that binds MHC class I molecules and delivers a negative signal inhibiting killing by natural killer and regulatory T cells; (v) LY75 (lymphocyte antigen 75) an endocytotic receptor used by dendritic cells to direct captured antigens from the extracellular space to a specialized antigen-processing compartment; and (vi) CDW52, a 21-28 kDa glycopeptide antigen expressed on lymphocytes and macrophages known to be a target for complement-mediated insult, inducing pro-inflammatory cytokine (e.g. TNF alpha and interferon gamma) production. Other up-regulated genes are members of the anti-apoptotic pathways, and include PIP5K1-gamma (Phosphatidylinositol-4-phosphate 5-kinase, type 1, gamma) and MAP4 (Microtubule-associated protein 4). Over-expression of transcripts belonging to the papain cysteine proteinase family CTSK (Cathepsin K) and CTSB (Cathepsin B) was also observed.

Down-regulated genes in MS—Abundant down-regulated transcripts unique to MS that were identified include IL1B (Interleukin 1 beta), an important inflammatory cytokine; TRAF6, which is essential for IL1 signaling; and SCYA20, known to be mediated by IL1B. Decreased mRNA expression of IL1B was strengthened by the down regulation of IL1R (type1 receptor), IL1RAP (receptor accessory protein) and IL1RN (receptor antagonist).

Other important down-regulated genes include TGFB1 (Transforming growth Factor beta 1) and SKI (v-ski sarcoma viral oncogene homologue) a component of TGFB signaling pathway, both known to inhibit cell proliferation. Thus, their under expression may contribute to autoreactive T cell expansion. Members of epidermal growth factor family such as VEGF (Vascular endothelial growth factor), IGFBP4 (Insulin-like growth factor binding protein 4) and EREG (epiregulin) were also down regulated. Additionally, mRNA expression of members of the steroid-thyroid receptors family including nuclear receptor subfamily 4, group A members 1, 2 and 3 (NR4A1, NR4A2, NR4A3) were significantly reduced. Down regulation of these genes may inhibit apoptosis through Fas ligand and tumor necrosis factor alpha or through early response of T-cell receptor induced apoptosis of thymocytes, thus mimicking positive selection.

Taken together, the identification of profiles of up- (overexpressed) and down regulated genes specific to MS indicates the suitability of the methods of the present invention for identifying validated and significant molecular signatures of PBMC gene expression in MS. While reducing the present invention to practice, it was observed that the specific disease related genes include transcripts involved in T cell activation and expansion and anti-apoptotic mediators, indicating failure of apoptosis-related elimination of autoreactive T cells.

### Example II

### Stage Specific Gene Expression Profiles of MS

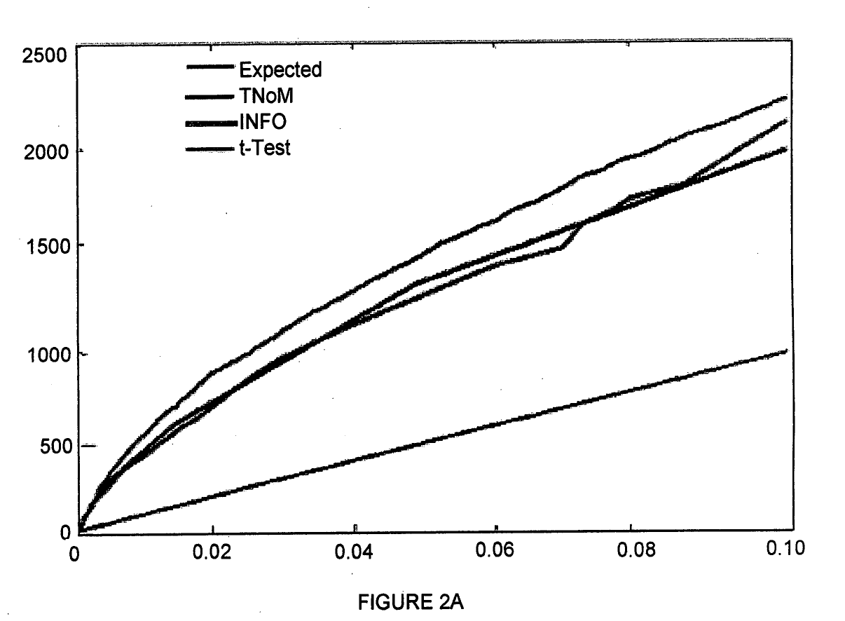

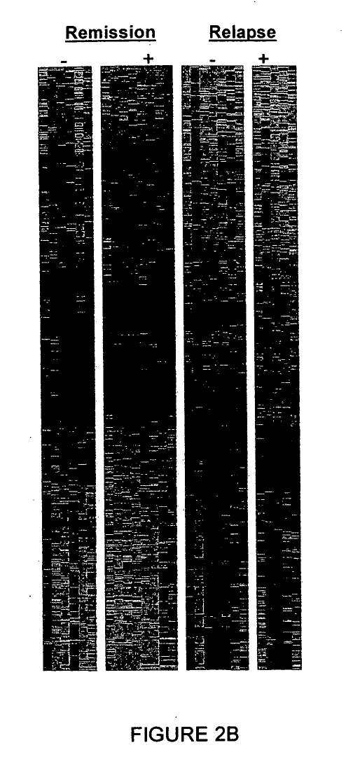

Accurate clinical tools for specific diagnosis of disease stages in MS are presently unavailable. In order to provide a useful profile of the clinically defined stages of MS, specific gene expression was evaluated in relation to clinical disease phases. Significant overabundance was found between the number of observed and expected genes expressed in MS patients during an acute relapse and in remission (FIG. 2A). Using the methods described hereinabove, the 743 most informative genes (302 up-regulated and 441 down-regulated) with p-value<0.05 in all three scores (t-test, TNoM, INFO) that differentiated relapse from remission (FIG. 2B, Table III) were identified.

Over-expressed genes in acute relapse of MS, compared to patients in remission—The most informative over-expressed genes included CTSL (Lysosomal cystein protease L, cathepsin L) known to play a role in MHC class II antigen presentation, responsible for quantitative and qualitative difference in peptide repertoires displayed by MHC class II molecules, and having a regulatory role in epitope generation for antigens subsets. Moreover, in vitro, proteolytic CTSL processed myelin basic protein into more then 60 different 20-40-mers species, and myelin-associated glycoprotein was described as a substrate for CTSL like proteases. These data, taken together with our observation that CTSL mRNA was over expressed in the active stage of MS, offer a biochemical basis for the immunodominant epitope spreading implicated in the pathogenesis of MS. Also up-regulated is SCYA2 (Monocyte specific chemoattractant protein, MCP1), essential for monocyte and NK cells recruitment to site of inflammatory injury. Augmented SCYA2 expression level in the CNS has been identified at the onset of EAE. Other abundant up-regulated transcripts identified by the method of the present invention include CD79A, DDIT3 (DNA-damage inducible transcript 3); E2-EPF (Ubiquitin carrier protein) and COX6.

Downregulated genes in acute relapse of MS, compared to patients in remission—From the downregulated gene transcripts in acute relapse vs. remission it is important to note several programmed cell death-related genes like CCNG1 (Cyclin G1) identified as p53 dependent apoptosis; PDCD2 (Programmed cell death 2) expressed in immature thymocytes; and CTLA1 (Cytotoxic T lymphocyte associated serine esterase 1), crucial for the rapid induction of apoptosis by cytotoxic cells. Also prominently down-regulated during acute relapse was JAK1 (Janus kinase 1), a protein tyrosine kinase reported to be obligatory for several cytokines receptors, important for regulation of acute cellular response.

The results of the functional annotation of the transcriptional motifs that distinguish between acute MS relapse and remission suggest that many of the genes are involved in cellular recruitment and epitope spreading, as well as important to immunologic mechanisms related to escape from regulatory surveillance and augmentation of cell survival potential. Thus, it can be suggested that during the acute inflammatory process of the disease there is a failure of the immune regulatory cells to inhibit autoreactivity and the self-expansion of the non-restrained autoreactive T cells further lead to a vicious cycle of on going inflammatory activity.

It is evident from the gene-clustering map (FIG. 2B) that during an acute relapse no significant differences are found between relapse treated vs. relapse untreated patients. Such a result is of great clinical significance, since this may indicate that during an acute MS exacerbation the major gene expression transcripts are related to relapse associated genes and the effect of therapy is negligible. However, during remission treatment effect was more pronounced and this effect on gene suppression in treated patients was evident.

Of even greater significance is the demonstration, for the first time, of a specific gene expression profile of the “probable” stage of MS. As described hereinabove, “probable” MS precedes definitive clinical diagnosis, and is characterized by diverse neurological symptoms including unilateral loss of vision, true vertigo, ataxia, paresthesia, incontinence, diplopia, dysarthria or paralysis. Probable MS patients may suffer undiagnosed for years. In order to provide a method for accurate diagnosis of probable MS, in advance of onset of clinical symptoms, gene expression in PBMC samples of 13 probable MS patients were compared with that of samples from 5 age-matched healthy controls. RNA preparation, hybridization to MicroArray and analysis of results was performed as described for Examples 1 and 2, and in the Material and Methods section hereinabove.

As is shown in Table V, a specific “probable” MS profile of gene expression distinguishes PBMCs of diseased and healthy individuals.

Thus, there is demonstrated, for the first time, gene expression profiles providing criteria for distinguishing between stages of MS in humans, for example, between relapsing and remitting MS, probable MS and healthy individuals. Further, the groups of up- and down-regulated genes identified herein may be used for investigation of mechanisms of disease and disease progression in MS.

### Example III

### Gene Expression Profiles in Treatment of MS

The effect of immunomodulatory treatment on gene expression in MS patients was investigated by comparison analysis of gene transcripts between treated and untreated patients. Suprisingly, despite the variety of immunomodulatory treatments and differences between patients in relation to treatment duration, the microarray methods described herein, treatment-related gene transcripts that differentiated between treated and untreated patients were detected. Treatment-specific gene expression is mainly associated with phosphorylation and signal transduction. Thus, gene microarray technology can be a powerful tool in evaluating and monitoring clinical correlations of effects of treatment, and determining prognosis.

Thus, data presented herein demonstrate for the first time distinct and significant fingerprint cluster in MS patients that differentiates them from healthy subjects. Moreover, the stringent and specific fingerprint is predictive for the diagnosis of MS and is suitable for guiding the selection of patients for early treatment. Additionally, separate gene expression patterns were identified between acute MS relapse and remission, and treatment effects could also be identified. The methods described herein may also be used to offer superior insight into the biological mechanisms involved in the disease as well as improving functional gene characterization and transcription sites detection, important for identification of new targets for treatment and drug identification, such as T cell activation and expansion and anti-apoptotic genes like SLAM, PIP5K1-g and the NR4A1-3 steroid-thyroid receptors subfamily.

### Example IV

Gene Expression Profiles of MOG-Reactive T-Cells from MS Patients

Although MS appears to be caused by autoimmune T cells activated against myelin self-antigens, myelin-reactive T-cells have been demonstrated in healthy subjects as well. Thus, distinction between disease-related and non-disease related T-cell myelin reactivity is of great clinical and investigational importance. In order to determine a profile of MS-related T-cell genes, gene expression in MOG-reactive T-cells from 4 MS patients having relapsing-remitting disease course, positive Poser criteria, and neurological disability, and 3 healthy age-matched controls was compared.

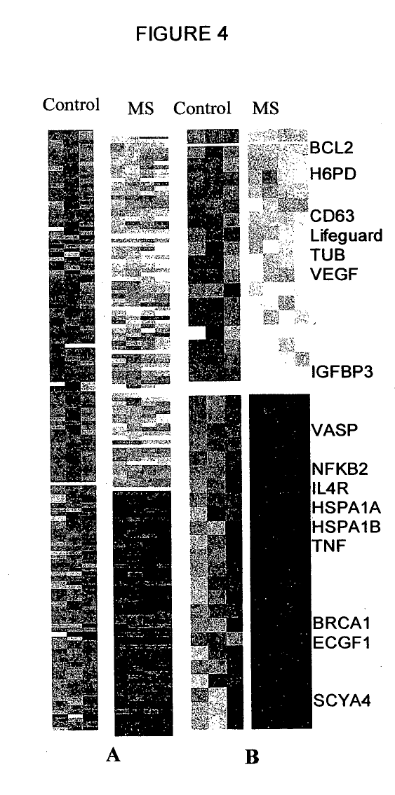

Using the microarray methods described herein, gene expression patterns obtained in MOG reactive T cell lines from MS patients detected 150 transcripts with TNoM=0, p=0.057 compared to healthy subjects (FIG. 4). These high scoring gene transcripts were defined as significant MOG reactive MS-related genes. Hierarchical clustering of gene expression patterns from MS patients and healthy controls is presented in FIG. 2, panel A. From the 150 genes with absolutely different expression levels, 43 most informative genes were further identified and clustered. These include 18 up-regulated and 25 down-regulated genes (FIG. 2, panel B).

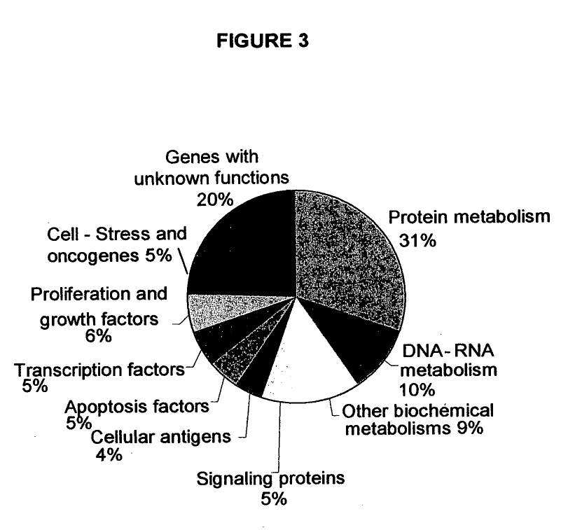

Investigation of the known biological function of these genes (Table V) shows a great diversity of activity (A Pie-chart diagram showing the functional groups of genes included in this evaluation is presented in FIG. 3). Included are genes coding for proteins involved in the regulation and execution of apoptosis, growth factors, mediators of signal transduction pathways, molecules that participate in inflammation and also genes encoding heat shock proteins, transcription factors and components of different biochemical pathways.

Upregulated Genes in MS-Derived T-cells—Up-regulated in MS patient-derived T-cell lines are several anti-apoptotic genes such as BCL2, lifeguard, and the MAP-activated kinase MAP3K12. The BCL2 gene product is an important member of the anti-apoptotic proteins. Lifeguard (LFG), is a molecule that inhibits cell death mediated by the Fas (CD95) receptor through a unique mechanism that down regulates apoptotic signals from Fas and is associated with human autoimmune lymphoproliferative syndrome (ALPS) and in lymphoproliferative lupus-like syndrome in mice.

The MAP3K12 gene is associated with programmed cell death and encodes a polypeptide that catalyzes the phosphorylation of BAD, a member of the BCL2 anti-apoptosis protein family. Increased expression of IGFBP3 and VEGF was also demonstrated in MS-derived T cells. IGFBP-3 has been implicated in the expansion of disease related T-cell, associated with acute brain lesions of MS patients. Thus, in addition to increased survival potential, our findings suggest that autoreactive T cells in MS also have an expansion advantage compared with T cells from healthy individuals.

Furthermore, migration of autoimmune T cells into the brain would be expected to be assisted by over-expression of transcripts encoding for vascular endothelial growth factor (VEGF) in lines from MS patients. VEGF enhances vascular permeability and may facilitate migration of lymphocytes into the CNS and induction of inflammatory reactions in the brain.

Downregulated Genes in MS-Derived T-cells—The profile of gene expression in MS-derived T-cells (FIG. 4, and Table V) indicates a suppression of apoptosis-related functions in the diseased state. One aspect of failure to induce apoptosis in the MS-derived T cell lines is the significant down-regulation of the gene encoding for the pro-apoptotic molecule TNF. A reduction in TNF could also contribute to a reduction in the ratio of pro- and anti-apoptotic transcript expression in the anti-MOG T cell lines from MS patients compared to healthy controls. Indeed, inadequate apoptosis present in MS autoreactive T cell lines could lead to insufficient deletion of autoimmune activated T cell clones and increase susceptibility to autoimmunity.

In addition, effectors of MHC class I presentation were revealed to be down-regulated in MS patients' cells. Such down-regulated expression includes the transcript for the proteasome PA28 complex, known to be a principal provider of MHC class I-presented peptides in antigen presenting cells, and HSP70 1A and 1B variants. TNF is also known to stimulate MHC class I presentation in addition to induction of apoptosis. The findings presented herein indicate that a weaker antigenic MHC class I presenting capability might distinguish MS-patient derived T cell lines from their healthy counterparts, and providing powerful diagnostic tools. It is conceivable that a lower expression of MHC class-I on CD4 autoimmune T cells might enable them to escape regulation by CD8 cells that recognize autoimmune idiotypes.

Taken together the combined effects of down-regulation of apoptosis associated genes, up regulation of anti-apoptotic genes, increased expansion capability by autoreactive T cells and enhanced ability to penetrate the CNS may lead to perpetuated pathologic cellular proliferation and tissue destruction within the CNS characteristic of MS, along with increased resistance to regulation. The specific gene expression profiles described herein can define some of the requirements for an individual to develop MS, and thus have important predictive value, especially in determining MS in the “probable” stage. It is noteworthy that despite activation in vitro with the same MOG epitope, anti-MOG T cells from healthy subjects did not attain the gene expression profile that characterized the MS patient-derived cells. The findings support the concept that not all autoimmune T cells are equal; autoimmune T cells from MS patients follow a unique pattern of T cell activation that appears to be more resilient to apoptosis and can support long term survival. Although T cell lines derived from MS patients and healthy donors responded to the same autoantigen, were both activated T cell populations that proliferated extensively in the presence of IL-2, the gene expression imprints that are unique to each group were preserved. These findings indicate the existence of different T-cell activation mechanisms. The nature of the stimuli that generate aberrant autoimmune T-cell gene expression has yet to be identified in order to determine whether their formation is merely the result of the chronic immune stimulation driven by other factors in MS, or whether such T cells function as primary drivers of the MS process. Characterization of such driver T cells, dictating the state of immunity/autoimmunity can also greatly contribute to understanding autoimmunity and possibly also for designing effective treatments for MS.

TABLE I
Gene Expression Profile from PBMCs of MS vs. Healthy

| | | Info | t-Test | Log Fold | |
|---|---|---|---|---|---|
| Identifier | TNOM PValue | PValue | PValue | Change | Symbol |
| U78107 | 8.55E−11 | 1.94E−11 | 4.04E−12 | −0.43769 | NAPG |
| M15330 | 8.55E−11 | 8.55E−11 | 2.49E−12 | −2.13825 | IL1B |
| X15218 | 8.55E−11 | 8.55E−11 | 1.4E−10 | −1.41501 | SKI |
| AF024710 | 8.55E−11 | 8.55E−11 | 1.13E−12 | −1.95537 | VEGF |
| U09937 | 1.84E−09 | 4.16E−10 | 2.04E−09 | −1.21578 | HSUROKR7 |
| AB018343 | 1.84E−09 | 4.16E−10 | 9.05E−12 | 0.383078 | KIAA0800 |
| X74039 | 1.84E−09 | 4.16E−10 | 1.51E−10 | −0.67381 | PLAUR |
| M64571 | 1.84E−09 | 1.84E−09 | 2.41E−11 | 0.416659 | MAP4 |
| U64197 | 1.84E−09 | 1.84E−09 | 2.95E−10 | −0.62373 | SCYA20 |
| X68452 | 2.57E−08 | 2.93E−09 | 9.12E−11 | −0.26618 | CCND2 |
| AB011161 | 2.57E−08 | 2.93E−09 | 9.64E−11 | 0.63432 | PIP5K1C |
| L47738 | 2.57E−08 | 2.93E−09 | 7.54E−09 | 0.31646 | PIR121 |
| U78798 | 2.57E−08 | 2.93E−09 | 1.11E−06 | −0.3172 | TRAF6 |
| M63904 | 2.57E−08 | 7.16E−09 | 5.38E−09 | −0.59612 | GNA15 |
| U72066 | 2.57E−08 | 7.16E−09 | 4.33E−08 | −0.34482 | RBBP8 |
| AI184802 | 2.64E−07 | 1.61E−08 | 2.67E−09 | −0.21576 | HPRP4P |
| AF077820 | 2.64E−07 | 1.61E−08 | 2.91E−08 | 0.656852 | LRP5 |
| L13740 | 2.64E−07 | 1.61E−08 | 5.83E−08 | −1.45891 | NR4A1 |
| AL008583 | 2.64E−07 | 1.61E−08 | 1.12E−08 | 0.250082 | |
| Z24724 | 2.64E−07 | 1.61E−08 | 5.96E−09 | −1.10426 | |
| D30783 | 2.57E−08 | 2.19E−08 | 8.95E−10 | −1.65011 | EREG |
| U47927 | 2.57E−08 | 2.19E−08 | 5.53E−09 | 0.545592 | USP5 |
| AI560890 | 2.57E−08 | 2.19E−08 | 1.8E−07 | 0.179028 | |
| Y00630 | 2.57E−08 | 3.69E−08 | 6.65E−09 | −2.38485 | SERPINB2 |
| N90866 | 2.64E−07 | 8.23E−08 | 2.76E−08 | 0.304525 | CDW52 |
| AF022375 | 2.64E−07 | 8.23E−08 | 1.87E−11 | −1.35847 | VEGF |
| M24895 | 2.11E−06 | 1.08E−07 | 1.72E−08 | 0.476779 | AMY2B |
| AF054176 | 2.11E−06 | 1.08E−07 | 6.47E−09 | −0.58138 | C1orf7 |
| L20941 | 2.64E−07 | 1.08E−07 | 1.78E−06 | −0.58618 | FTH1 |
| L05424 | 2.11E−06 | 1.08E−07 | 2.27E−09 | −0.58081 | HUMSCG19 |
| AB002347 | 2.11E−06 | 1.08E−07 | 7.19E−10 | 0.371731 | KIAA0349 |
| AB023153 | 2.11E−06 | 1.08E−07 | 1.82E−08 | 0.895842 | KIAA0936 |
| AF069517 | 2.11E−06 | 1.08E−07 | 4.91E−07 | 0.399638 | RBM6 |
| X69392 | 2.64E−07 | 1.08E−07 | 1.1E−08 | 0.297444 | RPL26 |
| U51920 | 2.11E−06 | 1.08E−07 | 7.01E−08 | −0.28142 | SRP54 |
| L22075 | 2.64E−07 | 1.71E−07 | 1.1E−08 | −0.55736 | GNA13 |
| X04500 | 2.64E−07 | 1.71E−07 | 3.43E−10 | −2.12121 | IL1B |
| AB028951 | 2.64E−07 | 1.71E−07 | 8.78E−09 | 0.543028 | KIAA1028 |
| AF004230 | 2.64E−07 | 1.71E−07 | 3.06E−07 | 0.349166 | LILRB1 |
| AF070582 | 2.64E−07 | 1.71E−07 | 3.23E−08 | −0.19773 | MGC13033 |
| X66363 | 2.64E−07 | 1.71E−07 | 6.53E−07 | −0.24505 | PCTK1 |
| L33881 | 2.64E−07 | 1.71E−07 | 5.06E−08 | −0.59585 | PRKCI |
| U33017 | 2.64E−07 | 1.71E−07 | 5.2E−07 | 0.373581 | SLAM |
| AJ007042 | 2.64E−07 | 1.71E−07 | 2.1E−07 | 0.170935 | WHSC1 |
| Z93930 | 2.64E−07 | 1.71E−07 | 2.42E−05 | −0.39839 | XBP1 |
| AF079167 | 2.64E−07 | 1.71E−07 | 7.37E−10 | −1.93249 | |
| AF098641 | 2.64E−07 | 1.71E−07 | 1.56E−07 | −0.41172 | |
| HG3227-HT3404 | 2.64E−07 | 1.71E−07 | 1.68E−08 | −0.25361 | |
| U78302 | 2.64E−07 | 1.71E−07 | 2.41E−08 | 0.329878 | |
| U91543 | 2.64E−07 | 2.49E−07 | 2.01E−07 | 0.478678 | CHD3 |
| M22919 | 2.64E−07 | 2.49E−07 | 9.52E−08 | −0.81053 | MYL6 |
| AB029015 | 2.64E−07 | 2.49E−07 | 5.37E−09 | 0.695063 | PLCE2 |
| Z11697 | 1.37E−05 | 4.08E−07 | 3.55E−06 | −1.21033 | CD83 |
| AL096780 | 1.37E−05 | 4.08E−07 | 2.13E−06 | 0.34487 | CHKL |
| U51205 | 1.37E−05 | 4.08E−07 | 2.65E−07 | −0.76279 | COP9 |
| Y08683 | 1.37E−05 | 4.08E−07 | 4.71E−06 | 0.492738 | CPT1B |
| S52028 | 2.11E−06 | 4.08E−07 | 9.62E−08 | −0.81662 | CTH |
| X63368 | 2.11E−06 | 4.08E−07 | 2.3E−08 | −0.55432 | DNAJB2 |
| M84443 | 1.37E−05 | 4.08E−07 | 4.08E−07 | 0.303567 | GALK2 |
| U32324 | 1.37E−05 | 4.08E−07 | 3.21E−08 | 0.334966 | IL11RA |
| AB011115 | 1.37E−05 | 4.08E−07 | 3.39E−07 | 0.382809 | KIAA0543 |
| AB014535 | 1.37E−05 | 4.08E−07 | 1.04E−06 | 0.285282 | KIAA0635 |
| X02152 | 1.37E−05 | 4.08E−07 | 4.63E−08 | −0.75601 | LDHA |
| AF007130 | 2.11E−06 | 4.08E−07 | 2.51E−06 | 0.391811 | LOC54104 |
| AF007151 | 1.37E−05 | 4.08E−07 | 3.25E−06 | 0.468343 | MMS19L |
| X82209 | 2.11E−06 | 4.08E−07 | 1.37E−09 | −0.45281 | MN1 |
| X79882 | 1.37E−05 | 4.08E−07 | 1.78E−07 | 0.520965 | MVP |
| U91616 | 1.37E−05 | 4.08E−07 | 1.27E−07 | −0.80419 | NFKBIE |
| U41815 | 1.37E−05 | 4.08E−07 | 2.16E−07 | −0.96931 | NUP98 |
| AB011108 | 1.37E−05 | 4.08E−07 | 4.39E−07 | 0.453498 | PRP4 |
| L40377 | 1.37E−05 | 4.08E−07 | 3.49E−07 | −0.79409 | SERPINB8 |
| X99656 | 1.37E−05 | 4.08E−07 | 1.68E−06 | −0.23553 | SH3GL1 |
| AJ010059 | 2.11E−06 | 4.08E−07 | 2.95E−06 | 0.2235 | SIT |
| J02973 | 1.37E−05 | 4.08E−07 | 2.93E−07 | −1.30804 | THBD |
| N90862 | 1.37E−05 | 4.08E−07 | 3.28E−08 | 0.43576 | VAMP8 |
| Y14768 | 1.37E−05 | 4.08E−07 | 7.26E−08 | 0.248383 | |
| U47414 | 2.11E−06 | 7.73E−07 | 2.31E−06 | 0.370736 | CCNG2 |
| AB002386 | 2.11E−06 | 7.73E−07 | 5.34E−09 | 0.586117 | EZH1 |
| U29344 | 2.11E−06 | 7.73E−07 | 2.35E−07 | −0.43842 | FASN |
| AF015553 | 2.11E−06 | 7.73E−07 | 2.61E−07 | 0.61214 | GTF2I |
| AB028981 | 2.11E−06 | 7.73E−07 | 5.34E−07 | 0.282288 | KIAA1058 |
| U29656 | 2.11E−06 | 7.73E−07 | 7.52E−08 | 0.353186 | NME3 |
| X00737 | 2.11E−06 | 7.73E−07 | 5.21E−08 | −0.67074 | NP |
| U29185 | 2.11E−06 | 7.73E−07 | 1.56E−07 | −1.08006 | PRNP |
| AB007960 | 2.11E−06 | 7.73E−07 | 7.96E−06 | 0.447772 | SH3GLB1 |
| U44839 | 2.11E−06 | 7.73E−07 | 2.54E−07 | −0.97008 | USP11 |
| U84007 | 7.44E−05 | 1.28E−06 | 0.000235 | 0.236422 | AGL |
| S78187 | 7.44E−05 | 1.28E−06 | 1.95E−05 | 0.203265 | CDC25B |
| X82153 | 7.44E−05 | 1.28E−06 | 2.27E−06 | 0.47844 | CTSK |
| AL050084 | 7.44E−05 | 1.28E−06 | 5.26E−05 | 0.509331 | DC8 |
| X62535 | 1.37E−05 | 1.28E−06 | 5.68E−07 | 0.243937 | DGKA |
| AB026436 | 7.44E−05 | 1.28E−06 | 0.000219 | −0.7589 | DUSP10 |
| M98833 | 7.44E−05 | 1.28E−06 | 1.52E−06 | 0.434288 | FL11 |
| AW051579 | 1.37E−05 | 1.28E−06 | 7.58E−07 | 0.593476 | FLJ10512 |
| X16706 | 7.44E−05 | 1.28E−06 | 1.23E−06 | −1.09747 | FOSL2 |
| U90917 | 1.37E−05 | 1.28E−06 | 3.89E−07 | 0.433406 | FOXM1 |
| M24194 | 7.44E−05 | 1.28E−06 | 4.38E−06 | 0.560895 | GNB2L1 |
| AJ002190 | 7.44E−05 | 1.28E−06 | 2.17E−08 | 0.33775 | GNPAT |
| X87949 | 7.44E−05 | 1.28E−06 | 4.05E−07 | −0.54468 | HSPA5 |
| U96876 | 7.44E−05 | 1.28E−06 | 3.54E−06 | −0.45317 | INSIG1 |
| AF038564 | 1.37E−05 | 1.28E−06 | 2.05E−07 | −0.40446 | ITCH |
| D80011 | 7.44E−05 | 1.28E−06 | 4.2E−07 | −0.35073 | KIAA0189 |
| AI950382 | 1.37E−05 | 1.28E−06 | 1.63E−07 | −0.74128 | KIAA0585 |
| AB023235 | 7.44E−05 | 1.28E−06 | 1.43E−05 | 0.311216 | KIAA1018 |
| AB029038 | 7.44E−05 | 1.28E−06 | 7.62E−05 | 0.364386 | KIAA1115 |
| U24166 | 7.44E−05 | 1.28E−06 | 7.52E−06 | −0.45293 | MAPRE1 |
| X61498 | 7.44E−05 | 1.28E−06 | 8.8E−07 | −0.49884 | NFKB2 |
| U12767 | 7.44E−05 | 1.28E−06 | 2.84E−07 | −1.23483 | NR4A3 |
| U85245 | 7.44E−05 | 1.28E−06 | 4.57E−07 | 0.365266 | PIP5K2B |
| U50928 | 7.44E−05 | 1.28E−06 | 4.72E−06 | 0.302213 | PKD2 |
| U13695 | 7.44E−05 | 1.28E−06 | 1.11E−05 | 0.805607 | PMS1 |
| AA203527 | 1.37E−05 | 1.28E−06 | 1.18E−07 | 0.281992 | RPP20 |
| J02939 | 7.44E−05 | 1.28E−06 | 2.16E−07 | −0.87844 | SLC3A2 |
| N30151 | 7.44E−05 | 1.28E−06 | 5.05E−05 | 0.393521 | STX16 |
| U52960 | 2.11E−06 | 1.28E−06 | 1.51E−07 | −0.84863 | SURB7 |
| AF030249 | 1.37E−05 | 1.28E−06 | 1.98E−07 | 0.534547 | |
| AL022398 | 7.44E−05 | 1.28E−06 | 8.09E−08 | 0.919627 | |
| HG1103-HT1103 | 1.37E−05 | 1.28E−06 | 1.16E−07 | −0.39165 | |
| D30758 | 2.11E−06 | 1.8E−06 | 1.58E−05 | 0.27738 | CENTB1 |
| U75968 | 2.11E−06 | 1.8E−06 | 4.36E−06 | 0.139542 | DDX11 |
| M69199 | 2.11E−06 | 1.8E−06 | 1.45E−07 | −1.9021 | G0S2 |
| U20982 | 2.11E−06 | 1.8E−06 | 1.2E−08 | −0.67125 | IGFBP4 |
| AF040707 | 2.11E−06 | 1.8E−06 | 3.57E−07 | 0.289845 | NPR2L |
| AB007927 | 2.11E−06 | 1.8E−06 | 2.12E−07 | 0.323787 | RERE |
| AA902713 | 2.11E−06 | 1.8E−06 | 1.44E−06 | 0.474378 | |
| U66063 | 2.11E−06 | 2.24E−06 | 4.7E−07 | 0.277185 | CAMK2G |
| D13891 | 2.11E−06 | 2.24E−06 | 4.57E−05 | −0.20577 | ID2 |
| AL050087 | 2.11E−06 | 2.24E−06 | 1.27E−07 | −0.31279 | KIAA1785 |
| N23137 | 2.11E−06 | 2.24E−06 | 2.06E−07 | 0.247311 | MPHOSPH9 |
| N42007 | 2.11E−06 | 2.24E−06 | 9.19E−05 | 0.167986 | NUP50 |
| M74525 | 2.11E−06 | 2.24E−06 | 3.5E−07 | −0.61792 | UBE2B |
| AF035281 | 2.11E−06 | 2.24E−06 | 4.87E−07 | 0.472445 | |
| U11732 | 1.37E−05 | 3.17E−06 | 3.04E−07 | −0.22574 | ETV6 |
| AB002348 | 1.37E−05 | 3.17E−06 | 2.49E−07 | 0.576346 | KIAA0350 |
| AB007891 | 1.37E−05 | 3.17E−06 | 3.99E−05 | 0.196376 | KIAA0431 |
| AI754391 | 1.37E−05 | 3.17E−06 | 1.72E−06 | −0.27657 | KLF12 |
| D50406 | 1.37E−05 | 3.17E−06 | 2.65E−05 | 0.461907 | RECK |
| AF070617 | 1.37E−05 | 3.17E−06 | 3.23E−07 | 0.323494 | |
| M23114 | 2.11E−06 | 4.08E−06 | 1.59E−07 | −0.96141 | ATP2A2 |
| AF014958 | 2.11E−06 | 4.08E−06 | 1.05E−07 | −0.42152 | CCRL2 |
| AF067853 | 1.37E−05 | 4.31E−06 | 5.02E−06 | 0.361707 | ADSL |
| M73547 | 1.37E−05 | 4.31E−06 | 9.2E−08 | 0.438897 | D5S346 |
| W28319 | 1.37E−05 | 4.31E−06 | 1.5E−05 | 0.294631 | FBLN1 |
| AB007895 | 1.37E−05 | 4.31E−06 | 9.61E−07 | 0.186643 | KIAA0435 |
| AB014579 | 1.37E−05 | 4.31E−06 | 6.08E−08 | 0.367966 | MGEA5 |
| AF019083 | 1.37E−05 | 4.31E−06 | 8.34E−07 | 0.17011 | PTENP1 |
| AL080141 | 1.37E−05 | 4.31E−06 | 2.42E−07 | 0.330868 | SEC31B-1 |
| AF110377 | 1.37E−05 | 4.31E−06 | 3.05E−05 | 0.361232 | TRRAP |
| AB002448 | 1.37E−05 | 4.31E−06 | 2.45E−07 | 0.468926 | |
| AL049787 | 1.37E−05 | 4.31E−06 | 7.11E−06 | 0.311278 | |
| U50527 | 1.37E−05 | 4.31E−06 | 5.11E−06 | 0.416543 | |
| Z32860 | 1.37E−05 | 4.31E−06 | 7.81E−06 | 0.133192 | |
| AF094481 | 1.37E−05 | 5.01E−06 | 2.74E−07 | −0.29045 | CGGBP1 |
| U29171 | 1.37E−05 | 5.01E−06 | 1.1E−06 | −0.6032 | CSNK1D |
| AL050196 | 1.37E−05 | 5.01E−06 | 2E−05 | −0.24688 | DKFZP586D2223 |
| U48807 | 1.37E−05 | 5.01E−06 | 4.97E−08 | −0.93178 | DUSP4 |
| U15552 | 1.37E−05 | 5.01E−06 | 1.67E−05 | −0.68094 | HSU15552 |
| L13740 | 1.37E−05 | 5.01E−06 | 9.1E−08 | −0.61928 | NR4A1 |
| AF010309 | 1.37E−05 | 5.01E−06 | 7.36E−07 | −0.28533 | PIG3 |
| Y18004 | 1.37E−05 | 5.01E−06 | 4.19E−07 | −0.9465 | SCML2 |
| R90942 | 1.37E−05 | 5.01E−06 | 1.05E−05 | −0.17696 | ST6GALNACIV |
| W28612 | 1.37E−05 | 5.01E−06 | 1.7E−06 | −0.25519 | |
| X64330 | 7.44E−05 | 6.03E−06 | 2.27E−06 | 0.297851 | ACLY |
| U49844 | 7.44E−05 | 6.03E−06 | 3.67E−07 | 0.47168 | ATR |
| AB015019 | 7.44E−05 | 6.03E−06 | 2.75E−07 | −0.24515 | BAIAP2 |
| AF006513 | 0.000344 | 6.03E−06 | 4.48E−05 | −1.45973 | CHD1 |
| U56998 | 0.000344 | 6.03E−06 | 3.7E−06 | −0.74294 | CNK |
| S68134 | 0.000344 | 6.03E−06 | 8.37E−07 | −1.64652 | CREM |
| S68134 | 0.000344 | 6.03E−06 | 4.35E−06 | −2.47105 | CREM |
| S68271 | 0.000344 | 6.03E−06 | 3.03E−06 | −2.07185 | CREM |
| AF021819 | 0.000344 | 6.03E−06 | 4.41E−05 | 0.298771 | DJ-1 |
| AF029777 | 1.37E−05 | 6.03E−06 | 8.27E−07 | 0.290159 | GCN5L2 |
| U28811 | 0.000344 | 6.03E−06 | 1.33E−06 | 0.32855 | GLG1 |
| S81914 | 0.000344 | 6.03E−06 | 4.18E−07 | −1.59146 | IER3 |
| X80821 | 0.000344 | 6.03E−06 | 8.51E−05 | −0.5606 | KIAA0874 |
| L06895 | 7.44E−05 | 6.03E−06 | 1.12E−05 | −0.1928 | MAD |
| D78579 | 1.37E−05 | 6.03E−06 | 4.25E−07 | −1.65638 | NR4A3 |
| D78579 | 7.44E−05 | 6.03E−06 | 9.62E−07 | −1.61438 | NR4A3 |
| U12767 | 0.000344 | 6.03E−06 | 2.55E−07 | −2.13744 | NR4A3 |
| M95678 | 0.000344 | 6.03E−06 | 2E−06 | 0.432923 | PLCB2 |
| X51804 | 0.000344 | 6.03E−06 | 7.23E−05 | −0.19283 | PMI |
| W28743 | 0.000344 | 6.03E−06 | 2.78E−06 | −0.28926 | PP1628 |
| X17042 | 7.44E−05 | 6.03E−06 | 6.64E−06 | −0.36481 | PRG1 |
| M80244 | 0.000344 | 6.03E−06 | 2.72E−06 | −0.8522 | SLC7A5 |
| AF001294 | 1.37E−05 | 6.03E−06 | 1.23E−06 | −0.76359 | TSSC3 |
| D49677 | 7.44E−05 | 6.03E−06 | 4.18E−06 | 0.198707 | U2AF1RS2 |
| AB011004 | 0.000344 | 6.03E−06 | 1.41E−06 | −1.34073 | UAP1 |
| AB011113 | 1.37E−05 | 6.03E−06 | 3.74E−07 | 0.444795 | WDR7 |
| AC002394 | 0.000344 | 6.03E−06 | 0.001473 | 0.17105 | |
| AL021707 | 0.000344 | 6.03E−06 | 4.95E−06 | −2.21462 | |
| AL022398 | 7.44E−05 | 6.03E−06 | 1.1E−07 | 0.79713 | |
| AL049442 | 0.000344 | 6.03E−06 | 8.09E−06 | 0.621935 | |
| U17760 | 0.000344 | 6.03E−06 | 4.25E−06 | −0.84472 | |
| L22569 | 1.37E−05 | 8.66E−06 | 1.52E−06 | 0.318129 | CTSB |
| AL031058 | 1.37E−05 | 8.66E−06 | 0.000375 | 0.149046 | DSP |
| AL080172 | 1.37E−05 | 8.66E−06 | 1.89E−05 | 0.098968 | FLJ21919 |
| M36821 | 1.37E−05 | 8.66E−06 | 2.21E−07 | −0.36334 | GRO3 |
| U06631 | 1.37E−05 | 8.66E−06 | 1.31E−05 | 0.486332 | H326 |
| L16499 | 1.37E−05 | 8.66E−06 | 5.12E−06 | 0.374296 | HHEX |
| X53586 | 1.37E−05 | 8.66E−06 | 3.4E−07 | 0.51291 | ITGA6 |
| D87466 | 1.37E−05 | 8.66E−06 | 1.49E−07 | 0.466046 | KIAA0276 |
| N98667 | 1.37E−05 | 8.66E−06 | 3.38E−07 | 0.367127 | KIAA1696 |
| X99142 | 1.37E−05 | 8.66E−06 | 1.24E−06 | −0.29773 | KRTHB6 |
| AF011333 | 1.37E−05 | 8.66E−06 | 1.55E−05 | 0.342503 | LY75 |
| U70735 | 1.37E−05 | 8.66E−06 | 1.82E−06 | 0.249185 | MOV34-34KD |
| U02020 | 1.37E−05 | 8.66E−06 | 1.37E−06 | −1.13863 | PBEF |
| M31724 | 1.37E−05 | 8.66E−06 | 0.000172 | −0.2601 | PTPN1 |
| U29175 | 1.37E−05 | 8.66E−06 | 1.9E−06 | 0.266342 | SMARCA4 |
| AL031846 | 1.37E−05 | 8.66E−06 | 0.000418 | 0.38404 | |
| Y12059 | 7.44E−05 | 1.51E−05 | 5.64E−06 | −0.46008 | BRD4 |
| U49187 | 7.44E−05 | 1.51E−05 | 1.48E−06 | 0.671467 | C6orf32 |
| X66945 | 7.44E−05 | 1.51E−05 | 1.91E−07 | −0.35494 | FGFR1 |
| M60922 | 7.44E−05 | 1.51E−05 | 4.47E−08 | 0.39657 | FLOT2 |
| AL049409 | 7.44E−05 | 1.51E−05 | 1.1E−06 | 0.714173 | LEF1 |
| L16794 | 7.44E−05 | 1.51E−05 | 2.23E−05 | −0.27553 | MEF2D |
| U77735 | 7.44E−05 | 1.51E−05 | 5.66E−06 | 0.574142 | PIM2 |
| U10117 | 7.44E−05 | 1.51E−05 | 4.07E−06 | 0.563673 | SCYE1 |
| AF023614 | 1.37E−05 | 1.51E−05 | 4.79E−07 | −0.20744 | TACI |
| S73591 | 1.37E−05 | 1.51E−05 | 4.68E−06 | 0.414777 | VDUP1 |
| AF052160 | 7.44E−05 | 1.51E−05 | 1.67E−06 | 0.623021 | |
| L76528 | 7.44E−05 | 1.51E−05 | 6.14E−06 | −0.39652 | |
| U51007 | 7.44E−05 | 1.51E−05 | 1.49E−06 | 0.309996 | |
| D10704 | 1.37E−05 | 1.75E−05 | 4.69E−07 | −0.36791 | CHK |
| U97105 | 1.37E−05 | 1.75E−05 | 6.56E−07 | 1.00615 | DPYSL2 |
| U03634 | 1.37E−05 | 1.75E−05 | 1E−06 | −0.21467 | LBC |
| L13773 | 1.37E−05 | 1.75E−05 | 6.44E−07 | 0.247919 | MLLT2 |
| M31523 | 1.37E−05 | 1.75E−05 | 2.09E−06 | 0.36898 | TCF3 |
| AL023553 | 1.37E−05 | 1.75E−05 | 2.51E−06 | 0.226635 | |
| W25984 | 7.44E−05 | 2.35E−05 | 1.42E−05 | 0.482493 | ACTA1 |
| U78521 | 0.000344 | 2.35E−05 | 2.53E−05 | 0.320909 | AIP |
| M30704 | 0.000344 | 2.35E−05 | 1.65E−05 | −0.37795 | AREG |
| X91504 | 0.001377 | 2.35E−05 | 0.00016 | 0.233217 | ARFRP1 |
| U51478 | 7.44E−05 | 2.35E−05 | 6.1E−07 | −0.58 | ATP1B3 |
| U21551 | 0.001377 | 2.35E−05 | 7.6E−05 | −0.3088 | BCAT1 |
| AB004066 | 0.000344 | 2.35E−05 | 6.57E−05 | −0.60905 | BHLHB2 |
| M59040 | 0.001377 | 2.35E−05 | 2.82E−06 | −0.46271 | CD44 |
| M91670 | 0.001377 | 2.35E−05 | 0.001649 | −0.47538 | E2-EPF |
| U43774 | 0.000344 | 2.35E−05 | 8.8E−07 | −0.39938 | FCAR |
| AW024285 | 0.000344 | 2.35E−05 | 6.99E−06 | −0.42098 | FLJ12443 |
| AA780049 | 7.44E−05 | 2.35E−05 | 7.39E−07 | 0.54912 | FLJ21439 |
| AI935146 | 0.000344 | 2.35E−05 | 2.05E−06 | −0.46726 | GALNT3 |
| AJ011679 | 0.001377 | 2.35E−05 | 4.67E−05 | 0.243248 | GAPCENA |
| AI670100 | 7.44E−05 | 2.35E−05 | 7.7E−07 | 0.22677 | GRLF1 |
| D87119 | 7.44E−05 | 2.35E−05 | 1.8E−06 | 0.425625 | GS3955 |
| M92432 | 0.000344 | 2.35E−05 | 4.31E−05 | 0.363033 | GUCY2D |
| D50405 | 0.001377 | 2.35E−05 | 0.000688 | 0.387926 | HDAC1 |
| U07563 | 7.44E−05 | 2.35E−05 | 4.91E−07 | −0.25016 | HSABLGR3 |
| Y10313 | 0.001377 | 2.35E−05 | 0.003201 | −0.35345 | IFRD1 |
| D63485 | 0.000344 | 2.35E−05 | 9.04E−05 | 0.31177 | IKKE |
| L08488 | 0.000344 | 2.35E−05 | 7.54E−06 | −0.37883 | INPP1 |
| X06256 | 1.37E−05 | 2.35E−05 | 4.89E−07 | −0.7357 | ITGA5 |
| D42084 | 0.001377 | 2.35E−05 | 7.39E−06 | 0.222195 | KIAA0094 |
| D43947 | 7.44E−05 | 2.35E−05 | 0.000104 | 0.269941 | KIAA0100 |
| AB007870 | 0.000344 | 2.35E−05 | 0.000108 | −0.64362 | KIAA0410 |
| AI950382 | 0.000344 | 2.35E−05 | 0.000122 | −0.65985 | KIAA0585 |
| AB014548 | 7.44E−05 | 2.35E−05 | 2.77E−05 | 0.431229 | KIAA0648 |
| AB018297 | 0.001377 | 2.35E−05 | 0.000836 | 0.195704 | KIAA0754 |
| AI970189 | 0.000344 | 2.35E−05 | 6.16E−07 | −0.75934 | KIAA0997 |
| L04733 | 0.001377 | 2.35E−05 | 8.84E−07 | 0.306455 | KNS2 |
| AF010193 | 7.44E−05 | 2.35E−05 | 1.26E−07 | −1.4705 | MADH7 |
| U18919 | 7.44E−05 | 2.35E−05 | 1.05E−05 | 0.271231 | NBP |
| U85430 | 0.001377 | 2.35E−05 | 0.000315 | 0.317554 | NFATC3 |
| S76638 | 7.44E−05 | 2.35E−05 | 7.47E−07 | −0.35416 | NFKB2 |
| AL050353 | 0.000344 | 2.35E−05 | 4.42E−06 | 0.179352 | OIP2 |
| L20971 | 0.001377 | 2.35E−05 | 0.00089 | −0.49725 | PDE4B |
| AF060502 | 7.44E−05 | 2.35E−05 | 0.000114 | −0.18239 | PEX10 |
| X80497 | 0.001377 | 2.35E−05 | 0.000245 | 0.313262 | PHKA2 |
| AL050371 | 0.000344 | 2.35E−05 | 3.7E−06 | 0.493288 | PISD |
| U77718 | 7.44E−05 | 2.35E−05 | 6.6E−06 | 0.352996 | PNN |
| U52427 | 0.001377 | 2.35E−05 | 0.000282 | 0.329478 | POLR2G |
| U94778 | 0.000344 | 2.35E−05 | 1.18E−05 | 0.282929 | PSTPIP1 |
| U48296 | 0.001377 | 2.35E−05 | 0.00011 | −0.89871 | PTP4A1 |
| M31166 | 0.001377 | 2.35E−05 | 0.000256 | −0.38484 | PTX3 |
| AJ001016 | 7.44E−05 | 2.35E−05 | 1.08E−05 | −0.28245 | RAMP3 |
| AF040965 | 0.001377 | 2.35E−05 | 0.001101 | −0.38591 | RES4-25 |
| J04130 | 0.000344 | 2.35E−05 | 3.02E−06 | −0.62071 | SCYA4 |
| U81800 | 0.000344 | 2.35E−05 | 4.28E−05 | −0.49523 | SLC16A3 |
| AB000734 | 0.001377 | 2.35E−05 | 0.000883 | −0.58764 | SSI-1 |
| U38847 | 7.44E−05 | 2.35E−05 | 9.91E−07 | 0.222946 | TARBP1 |
| M63180 | 0.001377 | 2.35E−05 | 1.03E−05 | −0.33301 | TARS |
| D15050 | 0.001377 | 2.35E−05 | 0.000192 | −1.12874 | TCF8 |
| M12959 | 7.44E−05 | 2.35E−05 | 1.61E−06 | 0.128482 | TRA@ |
| X00734 | 0.001377 | 2.35E−05 | 0.000384 | −0.34516 | TUBB5 |
| AJ001340 | 0.001377 | 2.35E−05 | 4.21E−05 | 0.181208 | U3-55K |
| Y08614 | 0.001377 | 2.35E−05 | 6.92E−05 | 0.305659 | XPO1 |
| AF054589 | 0.000344 | 2.35E−05 | 1.98E−06 | 0.945394 | |
| AL022398 | 7.44E−05 | 2.35E−05 | 2.4E−06 | 0.493166 | |
| AL031178 | 7.44E−05 | 2.35E−05 | 3.18E−05 | 0.410068 | |
| AL049782 | 7.44E−05 | 2.35E−05 | 7.66E−07 | 0.237794 | |
| HG1471-HT3923 | 0.001377 | 2.35E−05 | 0.000519 | 0.203133 | |
| HG4582-HT4987 | 7.44E−05 | 2.35E−05 | 4.63E−07 | −0.39588 | |
| U96629 | 0.001377 | 2.35E−05 | 1E−04 | 0.277256 | |
| D64110 | 7.44E−05 | 2.58E−05 | 7.49E−05 | −0.51036 | BTG3 |
| J04111 | 7.44E−05 | 2.58E−05 | 0.000108 | −1.60276 | JUN |
| J04111 | 7.44E−05 | 2.58E−05 | 4.68E−05 | −1.14014 | JUN |
| X56681 | 7.44E−05 | 2.58E−05 | 0.000112 | −0.48711 | JUND |
| D21853 | 7.44E−05 | 2.58E−05 | 0.000403 | −0.25594 | KIAA0111 |
| X80692 | 7.44E−05 | 2.58E−05 | 3.44E−05 | −1.1939 | MAPK6 |
| S76638 | 7.44E−05 | 2.58E−05 | 5.23E−05 | −0.46026 | NFKB2 |
| U65785 | 7.44E−05 | 2.58E−05 | 9.67E−06 | −0.2389 | ORP150 |
| AB016247 | 7.44E−05 | 2.58E−05 | 3.13E−05 | −0.57287 | SC5DL |
| M55153 | 7.44E−05 | 2.58E−05 | 4.77E−06 | −0.27465 | TGM2 |
| U02570 | 1.37E−05 | 2.81E−05 | 1.26E−06 | 0.432431 | ARHGAP1 |
| X04366 | 1.37E−05 | 2.81E−05 | 5.11E−06 | 0.346076 | CAPN1 |
| L10413 | 1.37E−05 | 2.81E−05 | 6.46E−06 | 0.207231 | FNTA |
| AF055001 | 1.37E−05 | 2.81E−05 | 9.78E−06 | −0.9457 | HERPUD1 |
| AI523538 | 1.37E−05 | 2.81E−05 | 0.004471 | −0.1584 | HIPK3 |
| X59373 | 1.37E−05 | 2.81E−05 | 1.31E−05 | −0.22992 | HOXD10 |
| X99209 | 1.37E−05 | 2.81E−05 | 2.65E−05 | 0.239777 | HRMT1L1 |
| M65217 | 1.37E−05 | 2.81E−05 | 1.02E−05 | 0.33377 | HSF2 |
| X17025 | 1.37E−05 | 2.81E−05 | 1.45E−05 | −0.44351 | IDI1 |
| M35878 | 1.37E−05 | 2.81E−05 | 4.29E−05 | −0.25267 | IGFBP3 |
| D63486 | 1.37E−05 | 2.81E−05 | 9.69E−06 | 0.235319 | KIAA0152 |
| AB002303 | 1.37E−05 | 2.81E−05 | 1.86E−05 | −0.39642 | KIAA0305 |
| U20816 | 1.37E−05 | 2.81E−05 | 4.92E−05 | −0.20145 | NFKB2 |
| M61906 | 1.37E−05 | 2.81E−05 | 5.93E−06 | −0.39875 | PIK3R1 |
| U13695 | 1.37E−05 | 2.81E−05 | 1.31E−05 | 0.362255 | PMS1 |
| U38979 | 1.37E−05 | 2.81E−05 | 3.95E−05 | 0.158105 | PMS2L9 |
| X70218 | 1.37E−05 | 2.81E−05 | 2.44E−06 | −0.74691 | PPP4C |
| AC002400 | 1.37E−05 | 2.81E−05 | 2.28E−06 | −0.25834 | |
| AC005390 | 1.37E−05 | 2.81E−05 | 2.99E−05 | −0.24231 | |
| AF070606 | 1.37E−05 | 2.81E−05 | 1.48E−06 | 0.89337 | |
| HG2724-HT2820 | 1.37E−05 | 2.81E−05 | 5.17E−06 | −1.33814 | |
| X84194 | 7.44E−05 | 4.67E−05 | 6.38E−05 | 0.23578 | ACYP1 |
| AF039656 | 0.00482 | 4.67E−05 | 0.000251 | −0.73273 | BASP1 |
| AB002384 | 0.00482 | 4.67E−05 | 4.22E−05 | 0.548091 | C6orf32 |
| X98172 | 7.44E−05 | 4.67E−05 | 5.29E−07 | 0.507556 | CASP8 |
| U60521 | 7.44E−05 | 4.67E−05 | 8.13E−06 | −0.36762 | CASP9 |
| U11791 | 0.00482 | 4.67E−05 | 0.000363 | −1.0232 | CCNH |
| U67615 | 0.00482 | 4.67E−05 | 0.000948 | 1.23433 | CHS1 |
| AF037339 | 0.000344 | 4.67E−05 | 1.59E−05 | −0.33549 | CLPTM1 |
| U65928 | 7.44E−05 | 4.67E−05 | 2.85E−07 | 0.408918 | COPS5 |
| U37408 | 7.44E−05 | 4.67E−05 | 3.06E−05 | 0.157458 | CTBP1 |
| AB023143 | 0.00482 | 4.67E−05 | 0.001982 | 0.215415 | DEFCAP |
| AB014888 | 0.001377 | 4.67E−05 | 0.000204 | −0.34841 | DNAJB6 |
| M60278 | 0.00482 | 4.67E−05 | 3.33E−05 | −0.9007 | DTR |
| U88629 | 0.000344 | 4.67E−05 | 9.58E−07 | −0.32607 | ELL2 |
| M31899 | 0.000344 | 4.67E−05 | 0.000339 | 0.274507 | ERCC3 |
| M94856 | 7.44E−05 | 4.67E−05 | 4.99E−06 | −0.23847 | FABP5 |
| X86779 | 0.001377 | 4.67E−05 | 1.08E−05 | 0.140032 | FASTK |
| L00634 | 0.00482 | 4.67E−05 | 0.00019 | 0.205256 | FNTA |
| AF078077 | 0.000344 | 4.67E−05 | 1.44E−05 | −1.47649 | GADD45B |
| D87119 | 7.44E−05 | 4.67E−05 | 4.62E−06 | 0.557116 | GS3955 |
| X17644 | 7.44E−05 | 4.67E−05 | 6.72E−06 | −0.71963 | GSPT1 |
| L19314 | 0.00482 | 4.67E−05 | 0.000922 | −0.35113 | HRY |
| U05681 | 7.44E−05 | 4.67E−05 | 3.37E−06 | −0.35383 | HSBCL3S2 |
| J00139 | 0.00482 | 4.67E−05 | 0.000196 | −0.12797 | HUMFOL5 |
| M24283 | 0.000344 | 4.67E−05 | 3.71E−06 | −1.32611 | ICAM1 |
| M62403 | 7.44E−05 | 4.67E−05 | 5.57E−07 | −0.53749 | IGFBP4 |
| M28130 | 7.44E−05 | 4.67E−05 | 8.02E−07 | −2.27292 | IL8 |
| Z56281 | 0.001377 | 4.67E−05 | 0.000243 | 0.309173 | IRF3 |
| L12002 | 7.44E−05 | 4.67E−05 | 1.23E−06 | 0.286717 | ITGA4 |
| K00558 | 0.001377 | 4.67E−05 | 0.002498 | 0.12909 | K-ALPHA-1 |
| AL044599 | 0.001377 | 4.67E−05 | 8.81E−05 | 0.321294 | KIAA0222 |
| AB002344 | 0.000344 | 4.67E−05 | 1.04E−05 | −0.39307 | KIAA0346 |
| AB007889 | 7.44E−05 | 4.67E−05 | 2.33E−05 | 0.255643 | KIAA0429 |
| AB007916 | 0.00482 | 4.67E−05 | 0.000147 | 0.493018 | KIAA0447 |
| AB014538 | 0.000344 | 4.67E−05 | 1.98E−06 | −0.63923 | KIAA0638 |
| AF055004 | 7.44E−05 | 4.67E−05 | 9.29E−05 | 0.200537 | KIAA0763 |
| AI148772 | 0.000344 | 4.67E−05 | 4.18E−06 | −1.02619 | KYNU |
| AF064491 | 0.00482 | 4.67E−05 | 0.000148 | −0.54215 | LDB1 |
| L78132 | 7.44E−05 | 4.67E−05 | 5.15E−07 | 0.358576 | LGALS8 |
| X83441 | 7.44E−05 | 4.67E−05 | 6.75E−06 | −0.17796 | LIG4 |
| AF055581 | 7.44E−05 | 4.67E−05 | 5.69E−06 | −1.05728 | LNK |
| AL049963 | 0.000344 | 4.67E−05 | 8.36E−07 | −0.74421 | LOC64116 |
| AF014837 | 0.00482 | 4.67E−05 | 0.000636 | 0.325349 | M6A |
| D14497 | 0.001377 | 4.67E−05 | 3.68E−05 | −0.58619 | MAP3K8 |
| X75346 | 7.44E−05 | 4.67E−05 | 1.99E−05 | −0.37877 | MAPKAPK2 |
| M62324 | 0.001377 | 4.67E−05 | 5.46E−05 | −0.44552 | MRF-1 |
| AB023208 | 0.000344 | 4.67E−05 | 1.37E−05 | 0.293901 | MSF |
| AF072928 | 0.001377 | 4.67E−05 | 1.13E−05 | −0.3089 | MTMR6 |
| AF045451 | 0.000344 | 4.67E−05 | 6.34E−06 | −0.40149 | NAB1 |
| M58603 | 7.44E−05 | 4.67E−05 | 1.28E−06 | −0.73537 | NFKB1 |
| U07132 | 0.00482 | 4.67E−05 | 0.001821 | −0.14679 | NR1H2 |
| X75918 | 7.44E−05 | 4.67E−05 | 3.5E−05 | −1.61126 | NR4A2 |
| S77154 | 0.00482 | 4.67E−05 | 0.000304 | −1.33785 | NR4A2 |
| AB020657 | 0.00482 | 4.67E−05 | 2.75E−05 | −0.50544 | NS1-BP |
| D88674 | 7.44E−05 | 4.67E−05 | 6.26E−06 | −0.99818 | OAZIN |
| U27459 | 0.00482 | 4.67E−05 | 3.39E−05 | 0.430016 | ORC2L |
| AF000545 | 7.44E−05 | 4.67E−05 | 3.48E−06 | −0.85393 | P2Y10 |
| AF005043 | 7.44E−05 | 4.67E−05 | 2.7E−06 | 0.408592 | PARG |
| AF026086 | 0.000344 | 4.67E−05 | 2.66E−06 | 0.297942 | PEX1 |
| AJ001625 | 7.44E−05 | 4.67E−05 | 9.91E−05 | 0.36837 | PEX3 |
| U30255 | 0.001377 | 4.67E−05 | 0.000826 | 0.325906 | PGD |
| M61906 | 0.000344 | 4.67E−05 | 0.000611 | −0.2492 | PIK3R1 |
| M60483 | 0.000344 | 4.67E−05 | 3.17E−05 | −0.32565 | PPP2CA |
| U14603 | 7.44E−05 | 4.67E−05 | 4.46E−05 | 0.427268 | PTP4A2 |
| AF069517 | 0.001377 | 4.67E−05 | 0.000441 | 0.330897 | RBM6 |
| M83221 | 0.000344 | 4.67E−05 | 1.58E−05 | −0.26782 | RELB |
| AF037195 | 0.00482 | 4.67E−05 | 8.27E−05 | 0.959619 | RGS14 |
| L07597 | 0.00482 | 4.67E−05 | 0.000169 | 0.277243 | RPS6KA1 |
| X15217 | 7.44E−05 | 4.67E−05 | 3.77E−07 | −0.2371 | SKIL |
| M20681 | 0.001377 | 4.67E−05 | 1.92E−05 | −0.99917 | SLC2A3 |
| AF030409 | 7.44E−05 | 4.67E−05 | 7.66E−06 | 0.412043 | SLC9A6 |
| AJ224358 | 0.00482 | 4.67E−05 | 0.009613 | 0.14432 | SURF5 |
| U49928 | 0.000344 | 4.67E−05 | 6.31E−06 | 0.352648 | TAB1 |
| X89750 | 7.44E−05 | 4.67E−05 | 7.38E−06 | −1.51687 | TGIF |
| AA453183 | 0.001377 | 4.67E−05 | 4.48E−05 | −0.61646 | TIM17 |
| M31165 | 7.44E−05 | 4.67E−05 | 1.38E−06 | −0.34617 | TNFAIP6 |
| AF064090 | 0.001377 | 4.67E−05 | 4.05E−05 | −0.38921 | TNFSF14 |
| AF082557 | 0.001377 | 4.67E−05 | 2.23E−06 | 0.226994 | TNKS |
| D87448 | 0.00482 | 4.67E−05 | 0.000735 | 0.468196 | TOPBP1 |
| X05276 | 0.00482 | 4.67E−05 | 8.97E−05 | −0.50457 | TPM4 |
| D50919 | 0.00482 | 4.67E−05 | 4.02E−05 | 0.332326 | TRIM14 |
| J03258 | 0.00482 | 4.67E−05 | 8.78E−05 | −0.33021 | VDR |
| AB007973 | 0.00482 | 4.67E−05 | 0.000146 | 0.271053 | |
| AF041081 | 0.00482 | 4.67E−05 | 5.92E−05 | 0.26539 | |
| AI889718 | 7.44E−05 | 4.67E−05 | 0.000143 | −0.15002 | |
| AL021154 | 0.000344 | 4.67E−05 | 2.19E−06 | −0.82935 | |
| AL049340 | 0.000344 | 4.67E−05 | 4.87E−05 | −0.91769 | |
| AL050078 | 0.000344 | 4.67E−05 | 1.63E−05 | −0.2875 | |
| AL050378 | 0.000344 | 4.67E−05 | 5.72E−06 | 0.360577 | |
| D50525 | 0.000344 | 4.67E−05 | 3.02E−06 | 0.486698 | |
| J04755 | 7.44E−05 | 4.67E−05 | 6.75E−05 | −0.37296 | |
| M60784 | 7.44E−05 | 4.67E−05 | 1.24E−06 | 0.559903 | |
| M63978 | 0.000344 | 4.67E−05 | 1.77E−06 | −0.44762 | |
| U90909 | 0.00482 | 4.67E−05 | 3.74E−05 | −0.64272 | |
| X63547 | 0.001377 | 4.67E−05 | 0.000303 | 0.505712 | |
| AA135683 | 0.000344 | 5.23E−05 | 0.000289 | −0.69258 | BASP1 |
| S78771 | 0.000344 | 5.23E−05 | 2.55E−06 | −0.31389 | BRD2 |
| AL080156 | 0.000344 | 5.23E−05 | 3.52E−05 | −0.94419 | DKFZP434J214 |
| D14838 | 0.000344 | 5.23E−05 | 7.34E−06 | −0.50648 | FGF9 |
| W28281 | 0.000344 | 5.23E−05 | 8.96E−06 | −1.09149 | GABARAIPL1 |
| AB002344 | 7.44E−05 | 5.23E−05 | 8.48E−07 | −1.00068 | KIAA0346 |
| U23070 | 0.000344 | 5.23E−05 | 3.62E−05 | −0.12321 | NMA |
| U04636 | 0.000344 | 5.23E−05 | 2.81E−06 | −1.85123 | PTGS2 |
| U47634 | 0.000344 | 5.23E−05 | 0.002405 | −0.21686 | TUBB4 |
| S73149 | 0.000344 | 5.23E−05 | 0.003714 | −0.15741 | |
| M63256 | 0.000344 | 5.92E−05 | 6.54E−07 | 0.454561 | CDR2 |
| U94905 | 0.000344 | 5.92E−05 | 2.08E−05 | 0.388608 | DGKZ |
| AF012023 | 7.44E−05 | 5.92E−05 | 1.02E−06 | 0.50623 | ICAP-1A |
| L10717 | 0.000344 | 5.92E−05 | 0.000158 | 0.345558 | ITK |
| D29642 | 0.000344 | 5.92E−05 | 8.3E−06 | 0.327019 | KIAA0053 |
| AB011128 | 0.000344 | 5.92E−05 | 0.000584 | 0.151161 | KIAA0556 |
| AF075587 | 0.000344 | 5.92E−05 | 7.55E−06 | 0.4405 | KIAA0916 |
| U66464 | 0.000344 | 5.92E−05 | 2.93E−05 | 0.255675 | MAP4K1 |
| U18919 | 0.000344 | 5.92E−05 | 0.000573 | 0.277847 | NBP |
| X58965 | 0.000344 | 5.92E−05 | 7.34E−05 | 0.231912 | NME2 |
| X13403 | 7.44E−05 | 5.92E−05 | 4.21E−07 | 0.146032 | POU2F1 |
| D89859 | 0.000344 | 5.92E−05 | 1.56E−05 | 0.375402 | ZFP161 |
| AF052100 | 0.000344 | 5.92E−05 | 1.37E−05 | 0.290021 | |
| N53547 | 7.44E−05 | 7.24E−05 | 1.8E−07 | 0.296678 | MGC5508 |
| L35013 | 0.000344 | 7.24E−05 | 0.000112 | −0.17331 | SF3B4 |
| Y17829 | 7.44E−05 | 7.24E−05 | 5.49E−06 | −0.6508 | SYN47 |
| AL049987 | 7.44E−05 | 7.24E−05 | 2.39E−05 | 0.193082 | |
| X66436 | 0.000344 | 7.24E−05 | 1.88E−06 | −0.26662 | |
| Z80345 | 7.44E−05 | 9.64E−05 | 7.31E−06 | 0.412137 | ACADS |
| U27467 | 7.44E−05 | 9.64E−05 | 5.65E−06 | −0.56637 | BCL2A1 |
| AI961669 | 7.44E−05 | 9.64E−05 | 0.000107 | −0.1656 | BIG2 |
| X61123 | 7.44E−05 | 9.64E−05 | 4.17E−07 | −1.15256 | BTG1 |
| U49187 | 7.44E−05 | 9.64E−05 | 3.53E−06 | 0.511392 | C6orf32 |
| D13639 | 7.44E−05 | 9.64E−05 | 8.56E−06 | −0.64255 | CCND2 |
| AL035398 | 7.44E−05 | 9.64E−05 | 0.000153 | 0.353395 | CGI-51 |
| U15932 | 7.44E−05 | 9.64E−05 | 0.00031 | −1.26603 | DUSP5 |
| AD001530 | 7.44E−05 | 9.64E−05 | 3.06E−05 | −0.37019 | DXS9928E |
| Y07909 | 7.44E−05 | 9.64E−05 | 0.000161 | −0.23489 | EMP1 |
| W27152 | 7.44E−05 | 9.64E−05 | 0.000502 | 0.186359 | FLJ10569 |
| L17131 | 7.44E−05 | 9.64E−05 | 1.48E−05 | −0.24039 | HMGIY |
| X04430 | 7.44E−05 | 9.64E−05 | 4.15E−05 | −0.21816 | IL6 |
| AB014608 | 7.44E−05 | 9.64E−05 | 4.59E−06 | 0.41494 | KIAA0708 |
| AF061258 | 7.44E−05 | 9.64E−05 | 1.58E−06 | 0.622201 | LIM |
| U90919 | 7.44E−05 | 9.64E−05 | 7.23E−06 | −0.50014 | LOC57862 |
| J05037 | 7.44E−05 | 9.64E−05 | 0.000185 | −0.19243 | SDS |
| X70944 | 7.44E−05 | 9.64E−05 | 2.08E−05 | −0.72892 | SFPQ |
| L41887 | 7.44E−05 | 9.64E−05 | 6.74E−06 | −0.52203 | SFRS7 |
| X59871 | 7.44E−05 | 9.64E−05 | 1.91E−05 | 0.376648 | TCF7 |
| AI742846 | 7.44E−05 | 9.64E−05 | 0.000374 | −0.48069 | VAPA |
| HG2007-HT2056 | 7.44E−05 | 9.64E−05 | 4.01E−06 | −0.41408 | |
| X58141 | 7.44E−05 | 9.64E−05 | 1.75E−06 | 0.384254 | |
| AB018323 | 7.44E−05 | 0.000106 | 2.41E−05 | 0.432301 | GASC1 |
| AB023192 | 7.44E−05 | 0.000106 | 0.000138 | 0.196185 | I-1 |
| AB020638 | 7.44E−05 | 0.000106 | 5.26E−05 | 0.233629 | KIAA0831 |
| U49395 | 7.44E−05 | 0.000106 | 0.001916 | 0.169175 | P2RX5 |
| M23379 | 7.44E−05 | 0.000106 | 3.22E−05 | 0.42571 | RASA1 |
| AF034176 | 7.44E−05 | 0.000106 | 0.000333 | 0.332105 | |
| AJ012755 | 7.44E−05 | 0.000106 | 0.000296 | 0.26445 | |
| AB007934 | 7.44E−05 | 0.000119 | 6.08E−06 | 0.345799 | ACF7 |
| M80899 | 7.44E−05 | 0.000119 | 2.48E−05 | 0.419409 | AHNAK |
| AB014529 | 7.44E−05 | 0.000119 | 1.84E−05 | 0.43403 | AKAP11 |
| U37547 | 7.44E−05 | 0.000119 | 6.74E−06 | −0.71736 | BIRC2 |
| U72649 | 7.44E−05 | 0.000119 | 0.000207 | −0.30079 | BTG2 |
| D49738 | 7.44E−05 | 0.000119 | 0.000136 | 0.292742 | CKAP1 |
| AJ006267 | 7.44E−05 | 0.000119 | 7.19E−06 | 0.427023 | CLPX |
| W28167 | 7.44E−05 | 0.000119 | 1.16E−05 | 0.214921 | COPS7A |
| U18300 | 7.44E−05 | 0.000119 | 2.43E−06 | 0.183171 | DDB2 |
| AI133727 | 7.44E−05 | 0.000119 | 1.43E−06 | 0.181464 | FLB6421 |
| AA526812 | 7.44E−05 | 0.000119 | 0.000106 | 0.259476 | FLJ10326 |
| D64142 | 7.44E−05 | 0.000119 | 1.66E−05 | 0.528036 | H1FX |
| U60319 | 7.44E−05 | 0.000119 | 0.001064 | 0.194324 | HFE |
| M17017 | 7.44E−05 | 0.000119 | 1.43E−06 | −1.74073 | IL8 |
| D32053 | 7.44E−05 | 0.000119 | 0.003279 | 0.222661 | KARS |
| AB007914 | 7.44E−05 | 0.000119 | 7.13E−05 | 0.302838 | KIAA0445 |
| U10485 | 7.44E−05 | 0.000119 | 7.4E−06 | 0.270352 | LRMP |
| U29656 | 7.44E−05 | 0.000119 | 4.31E−06 | 0.471876 | NME3 |
| AB014604 | 7.44E−05 | 0.000119 | 1.72E−05 | 0.425787 | OSBPL3 |
| U41745 | 7.44E−05 | 0.000119 | 0.00204 | 0.230274 | PDAP1 |
| S90469 | 7.44E−05 | 0.000119 | 5.56E−06 | −0.2636 | POR |
| M26683 | 7.44E−05 | 0.000119 | 3.7E−06 | −0.16179 | SCYA2 |
| X81789 | 7.44E−05 | 0.000119 | 2.23E−05 | 0.143079 | SF3A3 |
| L14595 | 7.44E−05 | 0.000119 | 3.55E−05 | −0.1953 | SLC1A4 |
| AL079286 | 7.44E−05 | 0.000119 | 0.000245 | 0.165851 | STAU2 |
| AA845349 | 7.44E−05 | 0.000119 | 7.78E−07 | 0.457176 | TRIP7 |
| X59303 | 7.44E−05 | 0.000119 | 0.000124 | 0.224891 | VARS2 |
| AB023219 | 7.44E−05 | 0.000119 | 1.41E−05 | 0.316475 | |
| M58603 | 7.44E−05 | 0.000129 | 9.08E−06 | −0.56835 | NFKB1 |
| X77723 | 7.44E−05 | 0.000129 | 0.006788 | −0.24317 | RAB5EP |
| AF117829 | 7.44E−05 | 0.000129 | 2.61E−06 | −0.57516 | RIPK2 |
| U52960 | 7.44E−05 | 0.000129 | 0.001042 | −0.24648 | SURB7 |
| U84011 | 0.00482 | 0.000149 | 0.000134 | 0.286331 | AGL |
| U90552 | 0.000344 | 0.000149 | 0.000182 | 0.288509 | BTN3A1 |
| M16336 | 0.00482 | 0.000149 | 0.000224 | 0.218007 | CD2 |
| U03106 | 0.000344 | 0.000149 | 0.000252 | −0.87784 | CDKN1A |
| AB009285 | 0.001377 | 0.000149 | 0.000137 | 0.235726 | CFDP1 |
| U63289 | 0.001377 | 0.000149 | 0.001722 | −0.43517 | CUGBP1 |
| AF000430 | 0.00482 | 0.000149 | 0.000694 | −0.19887 | DNM1L |
| L11329 | 0.001377 | 0.000149 | 0.000142 | −0.56584 | DUSP2 |
| AB007619 | 0.00482 | 0.000149 | 0.002073 | 0.198391 | EBAG9 |
| X81625 | 0.00482 | 0.000149 | 6.92E−05 | −0.80689 | ETF1 |
| AL050128 | 0.000344 | 0.000149 | 1.81E−05 | 0.459416 | FAM8A1 |
| L49169 | 0.001377 | 0.000149 | 8.18E−05 | −2.09549 | FOSB |
| L25665 | 0.000344 | 0.000149 | 3.34E−06 | −0.4513 | GNL1 |
| AI494623 | 0.00482 | 0.000149 | 0.000304 | 0.187206 | HCDI |
| D89678 | 0.001377 | 0.000149 | 3.03E−05 | 0.197298 | HNRPDL |
| U07563 | 0.000344 | 0.000149 | 1.02E−05 | −0.23627 | HSABLGR3 |
| W28589 | 0.00482 | 0.000149 | 0.000129 | 0.170457 | HSPD1 |
| N29665 | 0.000344 | 0.000149 | 3.34E−05 | 0.593294 | KIAA0618 |
| AB023207 | 0.000344 | 0.000149 | 8.64E−06 | −0.4056 | KIAA0990 |
| AL079277 | 0.00482 | 0.000149 | 0.000161 | 0.200656 | LOC54103 |
| Z14138 | 0.001377 | 0.000149 | 0.000197 | −0.85008 | MAP3K8 |
| N23137 | 0.001377 | 0.000149 | 4.12E−06 | 0.244083 | MPHOSPH9 |
| AF050640 | 0.001377 | 0.000149 | 6.03E−05 | 0.324021 | NDUFS2 |
| AF069987 | 0.001377 | 0.000149 | 4.44E−05 | 0.203382 | NIT1 |
| AF043325 | 0.000344 | 0.000149 | 1.06E−05 | 0.328186 | NMT2 |
| M10901 | 0.001377 | 0.000149 | 1.91E−05 | −0.58982 | NR3C1 |
| M12267 | 0.000344 | 0.000149 | 4.07E−06 | −0.3279 | OAT |
| U02882 | 0.00482 | 0.000149 | 0.000223 | −0.99878 | PDE4D |
| AF059531 | 0.000344 | 0.000149 | 6.73E−06 | 0.546441 | PRMT3 |
| M29893 | 0.001377 | 0.000149 | 9.96E−05 | −0.15688 | RALA |
| AB029028 | 0.001377 | 0.000149 | 9.11E−06 | 0.482258 | RAP140 |
| AB007448 | 0.00482 | 0.000149 | 0.000777 | −0.319 | SLC22A4 |
| D87969 | 0.00482 | 0.000149 | 0.001578 | 0.401991 | SLC35A1 |
| U66615 | 0.00482 | 0.000149 | 0.000196 | 0.235993 | SMARCC1 |
| U46691 | 0.00482 | 0.000149 | 1.48E−05 | −0.85179 | SUPT6H |
| AF049910 | 0.00482 | 0.000149 | 0.000373 | −0.32787 | TACC1 |
| X14787 | 0.001377 | 0.000149 | 2.88E−05 | −0.19161 | THBS1 |
| AI375913 | 0.00482 | 0.000149 | 0.000905 | −0.12102 | TOP2A |
| X02344 | 0.001377 | 0.000149 | 0.00414 | −0.20405 | TUBB2 |
| AF104421 | 0.000344 | 0.000149 | 6.5E−06 | 0.349373 | UROD |
| J03258 | 0.000344 | 0.000149 | 1.21E−06 | −0.58295 | VDR |
| M58297 | 0.000344 | 0.000149 | 1.57E−05 | 0.185829 | ZNF42 |
| Y11681 | 0.000344 | 0.000149 | 1.92E−05 | 0.234481 | |
| AF104942 | 0.001377 | 0.00019 | 4.83E−05 | 0.464438 | ABCC5 |
| L07261 | 0.000344 | 0.00019 | 0.003564 | 0.29763 | ADD1 |
| L19871 | 0.001377 | 0.00019 | 0.000105 | −0.19867 | ATF3 |
| J04027 | 0.000344 | 0.00019 | 0.000133 | −0.42466 | ATP2B1 |
| M83363 | 0.001377 | 0.00019 | 0.004471 | 0.177565 | ATP2B4 |
| AF038195 | 0.000344 | 0.00019 | 0.000134 | 0.281425 | BCS1L |
| S78771 | 0.001377 | 0.00019 | 0.000145 | −0.24109 | BRD2 |
| L07044 | 0.001377 | 0.00019 | 0.000284 | 0.186013 | CAMK2G |
| M28170 | 0.000344 | 0.00019 | 2.96E−05 | 0.356602 | CD19 |
| Y08682 | 0.000344 | 0.00019 | 0.000118 | 0.17398 | CPT1B |
| AF046059 | 0.001377 | 0.00019 | 0.000665 | 0.204072 | CREME9 |
| L06797 | 0.001377 | 0.00019 | 0.000455 | −0.93505 | CXCR4 |
| L39874 | 0.000344 | 0.00019 | 0.000354 | 0.353702 | DCTD |
| AC004475 | 0.000344 | 0.00019 | 2.86E−05 | 0.25205 | DKFZP434E2216 |
| AI538172 | 0.001377 | 0.00019 | 0.000621 | 0.243057 | DKFZp761B2423 |
| AF010187 | 0.000344 | 0.00019 | 1E−05 | 0.361895 | FIBP |
| AW051579 | 0.000344 | 0.00019 | 0.000258 | 0.390285 | FLJ10512 |
| M22632 | 0.001377 | 0.00019 | 1.34E−05 | 0.157239 | GOT2 |
| X59372 | 0.001377 | 0.00019 | 0.000528 | −0.12959 | HOXD9 |
| X12433 | 0.000344 | 0.00019 | 1.07E−05 | −0.39946 | HS1-2 |
| X15183 | 0.000344 | 0.00019 | 0.000645 | −0.22973 | HSPCA |
| AI912041 | 0.001377 | 0.00019 | 5.21E−05 | −0.38517 | HSPE1 |
| X75315 | 0.000344 | 0.00019 | 0.010841 | −0.64335 | HSRNASEB |
| L42324 | 0.000344 | 0.00019 | 0.000262 | −0.31758 | HUMFRCG |
| X69433 | 0.001377 | 0.00019 | 0.002925 | 0.209735 | IDH2 |
| Y00093 | 0.000344 | 0.00019 | 2.6E−05 | −0.39318 | ITGAX |
| M88458 | 0.001377 | 0.00019 | 0.002031 | −0.15998 | KDELR2 |
| AB011114 | 0.000344 | 0.00019 | 3.13E−05 | 0.278271 | KIAA0542 |
| AB011135 | 0.000344 | 0.00019 | 0.000149 | 0.247752 | KIAA0563 |
| U57721 | 0.001377 | 0.00019 | 3.47E−05 | −0.23188 | KYNU |
| Y11395 | 0.001377 | 0.00019 | 8.58E−05 | 0.34059 | LANCL1 |
| AI652660 | 0.000344 | 0.00019 | 2.28E−05 | 0.385107 | LOC51112 |
| AB026118 | 0.001377 | 0.00019 | 4.47E−06 | −0.24886 | MALT1 |
| AB011144 | 0.000344 | 0.00019 | 9.36E−05 | 0.26851 | MCM3AP |
| AI620381 | 0.000344 | 0.00019 | 8.06E−06 | 0.29605 | MGC3077 |
| AI525633 | 0.000344 | 0.00019 | 2.44E−05 | 0.170916 | MGC5576 |
| X16396 | 0.000344 | 0.00019 | 3.27E−06 | −0.6151 | MTHFD2 |
| V00568 | 0.000344 | 0.00019 | 0.000769 | 0.549224 | MYC |
| AL050281 | 0.000344 | 0.00019 | 2.85E−06 | 0.30517 | NAG |
| AI985272 | 0.000344 | 0.00019 | 0.000474 | −0.2571 | NMB |
| D38524 | 0.000344 | 0.00019 | 0.001313 | 0.228851 | NT5B |
| AJ225089 | 0.000344 | 0.00019 | 0.000531 | −0.2589 | OASL |
| Z82200 | 0.000344 | 0.00019 | 0.000136 | −0.28579 | P2Y10 |
| X63564 | 0.001377 | 0.00019 | 1.7E−05 | −0.28202 | POLR2A |
| S57501 | 0.001377 | 0.00019 | 0.002179 | 0.267744 | PPP1CA |
| X07109 | 0.000344 | 0.00019 | 0.000694 | 0.167774 | PRKCB1 |
| M28209 | 0.000344 | 0.00019 | 0.000392 | −0.52456 | RAB1 |
| M87339 | 0.000344 | 0.00019 | 3.41E−05 | 0.248151 | RFC4 |
| Z14000 | 0.000344 | 0.00019 | 3.91E−06 | −0.33734 | RING1 |
| X06815 | 0.000344 | 0.00019 | 3.5E−05 | 0.293968 | SNRP70 |
| L23959 | 0.000344 | 0.00019 | 1.82E−05 | −0.36834 | TFDP1 |
| AB018262 | 0.000344 | 0.00019 | 0.000241 | 0.319056 | TOMM70A |
| X00437 | 0.001377 | 0.00019 | 0.00022 | 0.248344 | TRB@ |
| AF061016 | 0.000344 | 0.00019 | 0.000264 | 0.349913 | UGDH |
| U62392 | 0.000344 | 0.00019 | 2.74E−05 | −0.65983 | ZNF193 |
| X78925 | 0.001377 | 0.00019 | 0.001253 | −0.28003 | ZNF267 |
| AI655015 | 0.001377 | 0.00019 | 0.00444 | 0.74681 | |
| AL049387 | 0.001377 | 0.00019 | 5.12E−06 | 0.379296 | |
| AL050376 | 0.000344 | 0.00019 | 0.00026 | 0.410405 | |
| AB008775 | 0.000344 | 0.000304 | 1.88E−06 | −0.80745 | AQP9 |
| AI141670 | 0.000344 | 0.000304 | 1.6E−06 | −0.2494 | CLCN2 |
| AL080071 | 0.000344 | 0.000304 | 3.12E−06 | 0.237367 | DKFZP564M082 |
| AB028964 | 0.000344 | 0.000304 | 5.07E−05 | 0.351352 | KIAA1041 |
| M16801 | 0.001377 | 0.000304 | 0.000458 | 0.412733 | NR3C2 |
| N36842 | 0.001377 | 0.000304 | 0.000577 | 0.172944 | UPF3A |
| AL096752 | 0.000344 | 0.000304 | 0.000323 | −0.20419 | |
| U76421 | 0.000344 | 0.000402 | 0.000278 | 0.226301 | ADARB1 |
| L13939 | 0.001377 | 0.000402 | 0.000215 | 0.180874 | AP1B1 |
| X97074 | 0.001377 | 0.000402 | 0.001924 | 0.298218 | AP2S1 |
| U72936 | 0.000344 | 0.000402 | 1.03E−05 | 0.356824 | ATRX |
| X94910 | 0.000344 | 0.000402 | 0.000204 | 0.249294 | C12orf8 |
| U18291 | 0.000344 | 0.000402 | 2.45E−05 | 0.594377 | CDC16 |
| L22005 | 0.001377 | 0.000402 | 0.000111 | −0.15257 | CDC34 |
| M59287 | 0.00482 | 0.000402 | 0.000276 | −0.72279 | CLK1 |
| U25435 | 0.000344 | 0.000402 | 0.000648 | 0.264876 | CTCF |
| L39874 | 0.000344 | 0.000402 | 2.41E−05 | 0.211923 | DCTD |
| X52104 | 0.000344 | 0.000402 | 0.000159 | 0.317963 | DDX5 |
| AL050062 | 0.000344 | 0.000402 | 0.000377 | 0.36401 | DKFZP566K023 |
| AL080081 | 0.00482 | 0.000402 | 0.000103 | −0.60871 | DNAJB9 |
| X63741 | 0.001377 | 0.000402 | 0.000175 | −0.59207 | EGR3 |
| D13988 | 0.001377 | 0.000402 | 0.000371 | 0.14676 | GDI2 |
| M27492 | 0.000344 | 0.000402 | 2.01E−06 | −0.32619 | IL1R1 |
| S66213 | 0.000344 | 0.000402 | 9.56E−05 | 0.247863 | ITGA6 |
| AJ005896 | 0.000344 | 0.000402 | 5.38E−05 | 0.210462 | JM4 |
| Y10745 | 0.00482 | 0.000402 | 6.39E−05 | −0.30524 | KCNJ15 |
| AB002374 | 0.00482 | 0.000402 | 0.000916 | 0.20284 | KIAA0376 |
| AB007874 | 0.001377 | 0.000402 | 0.000181 | −0.21662 | KIAA0414 |
| AB011133 | 0.00482 | 0.000402 | 0.000521 | 0.302843 | KIAA0561 |
| AB018335 | 0.00482 | 0.000402 | 9.03E−05 | 0.234274 | KIAA0792 |
| M13452 | 0.00482 | 0.000402 | 0.00148 | −0.28339 | LMNA |
| X68836 | 0.00482 | 0.000402 | 2.15E−05 | −0.57967 | MAT2A |
| U79256 | 0.000344 | 0.000402 | 2.24E−05 | 0.328028 | MGC14258 |
| X76538 | 0.001377 | 0.000402 | 5.62E−05 | 0.408464 | MPV17 |
| AB011093 | 0.000344 | 0.000402 | 0.000101 | 0.612928 | P114-RHO-GEF |
| X66363 | 0.001377 | 0.000402 | 6.8E−05 | −0.24041 | PCTK1 |
| U13695 | 0.00482 | 0.000402 | 2.31E−05 | 0.31531 | PMS1 |
| D87078 | 0.000344 | 0.000402 | 3.88E−05 | 0.497225 | PUM2 |
| Z97074 | 0.001377 | 0.000402 | 3.97E−05 | 0.296662 | RAB9P40 |
| X90530 | 0.000344 | 0.000402 | 3.52E−05 | 0.254197 | RAGB |
| U75679 | 0.001377 | 0.000402 | 0.000139 | −0.29594 | SLBP |
| AF007142 | 0.000344 | 0.000402 | 3.15E−06 | 0.678734 | |
| AL021977 | 0.00482 | 0.000402 | 8.82E−05 | −0.82538 | |
| AL080192 | 0.001377 | 0.000402 | 4.26E−05 | 0.201319 | |
| HG1980-HT2023 | 0.00482 | 0.000402 | 0.003775 | −0.48359 | |
| U47924 | 0.001377 | 0.000402 | 0.000134 | 0.52195 | |
| U83661 | 0.000344 | 0.000444 | 9.97E−06 | 0.270218 | ABCC5 |
| AI961929 | 0.000344 | 0.000444 | 1.88E−05 | 0.461528 | ARHGAP1 |
| X78817 | 0.000344 | 0.000444 | 2.82E−05 | 0.281835 | ARHGAP4 |
| AL080164 | 0.000344 | 0.000444 | 0.000218 | 0.268161 | DKFZP564C1940 |
| X90392 | 0.000344 | 0.000444 | 0.000208 | 0.150242 | DNASE1L1 |
| AI561196 | 0.000344 | 0.000444 | 0.000156 | 0.302434 | FLJ11806 |
| AJ008112 | 0.000344 | 0.000444 | 0.000246 | −0.32126 | FMNL |
| M94630 | 0.000344 | 0.000444 | 0.000244 | 0.274532 | HNRPD |
| M38180 | 0.000344 | 0.000444 | 0.003037 | −0.15741 | HSD3B1 |
| U79274 | 0.000344 | 0.000444 | 9.67E−05 | 0.285563 | HSU79274 |
| AB014585 | 0.000344 | 0.000444 | 6.48E−05 | 0.460196 | KIAA0685 |
| AB029001 | 0.000344 | 0.000444 | 0.000183 | −0.33324 | KIAA1078 |
| AA045160 | 0.000344 | 0.000444 | 4.62E−05 | 0.179556 | MRPS14 |
| M96824 | 0.000344 | 0.000444 | 2.18E−05 | 0.139326 | NUCB1 |
| Y10055 | 0.000344 | 0.000444 | 9.91E−06 | 0.176067 | PIK3CD |
| Z54367 | 0.000344 | 0.000444 | 1.3E−05 | −0.39738 | PLEC1 |
| AF014402 | 0.000344 | 0.000444 | 7.63E−05 | 0.147061 | PPAP2A |
| M30773 | 0.000344 | 0.000444 | 0.001161 | 0.41229 | PPP3R1 |
| M29386 | 0.000344 | 0.000444 | 6.26E−05 | −0.23554 | PRL |
| X02910 | 0.000344 | 0.000444 | 0.001378 | −0.17579 | TNF |
| S76792 | 0.000344 | 0.000444 | 0.000211 | −0.16737 | TNFRSF4 |
| Y09008 | 0.000344 | 0.000444 | 0.000352 | 0.168444 | UNG |
| U18009 | 0.000344 | 0.000444 | 0.002896 | 0.204706 | VATI |
| D14533 | 0.000344 | 0.000444 | 0.000837 | 0.246085 | XPA |
| W27419 | 0.000344 | 0.000444 | 2.83E−05 | −0.44121 | |
| Z85986 | 0.000344 | 0.000444 | 0.000407 | −0.2149 | |
| Z99716 | 0.000344 | 0.000444 | 5.14E−05 | 0.324642 | |
| U50939 | 0.001377 | 0.000525 | 1.19E−05 | 0.235552 | APPBP1 |
| Y15521 | 0.00482 | 0.000525 | 0.000492 | −0.28889 | ASMTL |
| J05682 | 0.00482 | 0.000525 | 0.000291 | −0.33004 | ATP6C |
| D26362 | 0.00482 | 0.000525 | 0.000707 | 0.247252 | BRD3 |
| AL120687 | 0.001377 | 0.000525 | 1.21E−05 | −0.55731 | CSH1 |
| U20350 | 0.00482 | 0.000525 | 0.010386 | 0.383475 | CX3CR1 |
| X04011 | 0.00482 | 0.000525 | 0.000154 | 0.348439 | CYBB |
| U78524 | 0.001377 | 0.000525 | 6.56E−05 | −0.36872 | DDXBP1 |
| U87947 | 0.001377 | 0.000525 | 5.05E−05 | −0.29673 | EMP3 |
| AL035252 | 0.00482 | 0.000525 | 0.003735 | 0.074075 | ENTPD6 |
| X04828 | 0.00482 | 0.000525 | 0.0015 | 0.256297 | GNAI2 |
| X56841 | 0.001377 | 0.000525 | 4.37E−05 | 0.338907 | HLA-E |
| D49410 | 0.00482 | 0.000525 | 0.00016 | −0.21753 | HUMIL3RA12 |
| L40586 | 0.00482 | 0.000525 | 3.02E−05 | −0.20891 | IDS |
| X52015 | 0.00482 | 0.000525 | 0.00032 | −0.54051 | IL1RN |
| D31888 | 0.00482 | 0.000525 | 7.5E−06 | −0.44687 | KIAA0071 |
| D42047 | 0.001377 | 0.000525 | 3.5E−05 | 0.226884 | KIAA0089 |
| AB007958 | 0.00482 | 0.000525 | 0.00023 | 0.259725 | KIAA0489 |
| AB011100 | 0.00482 | 0.000525 | 0.000193 | 0.418151 | KIAA0528 |
| AB014553 | 0.00482 | 0.000525 | 0.002002 | −0.25439 | KIAA0653 |
| AI888084 | 0.001377 | 0.000525 | 3.57E−05 | 0.391754 | KIAA1624 |
| X61118 | 0.00482 | 0.000525 | 0.001631 | 0.292879 | LMO2 |
| AJ004832 | 0.00482 | 0.000525 | 0.001715 | 0.30393 | NTE |
| AB020631 | 0.001377 | 0.000525 | 0.000212 | 0.379354 | PCF11 |
| AB002359 | 0.00482 | 0.000525 | 2.36E−05 | 0.271468 | PFAS |
| AB012229 | 0.001377 | 0.000525 | 0.000168 | −0.59579 | PFKFB3 |
| M83088 | 0.001377 | 0.000525 | 3.5E−05 | 0.439367 | PGM1 |
| X84908 | 0.001377 | 0.000525 | 2.25E−05 | 0.331887 | PHKB |
| U48250 | 0.001377 | 0.000525 | 8.51E−05 | −0.2234 | PRKCBP2 |
| AB007851 | 0.000344 | 0.000525 | 1.95E−05 | 0.481768 | PRPSAP2 |
| X97267 | 0.00482 | 0.000525 | 0.000303 | 0.211707 | PTPRCAP |
| M64595 | 0.00482 | 0.000525 | 0.004436 | 0.170959 | RAC2 |
| S59049 | 0.001377 | 0.000525 | 0.000915 | −0.61362 | RGS1 |
| AL050267 | 0.00482 | 0.000525 | 0.000204 | 0.311003 | SAMHD1 |
| W28498 | 0.00482 | 0.000525 | 1.7E−05 | −0.57386 | SAR1 |
| W27050 | 0.00482 | 0.000525 | 2.37E−05 | −0.587 | SFPQ |
| X92762 | 0.00482 | 0.000525 | 0.000116 | 0.283179 | TAZ |
| U18422 | 0.001377 | 0.000525 | 0.000279 | −0.14486 | TFDP2 |
| D87127 | 0.001377 | 0.000525 | 0.000126 | −0.32216 | TLOC1 |
| U12595 | 0.001377 | 0.000525 | 1.07E−05 | 0.347309 | TRAP1 |
| AF046024 | 0.00482 | 0.000525 | 0.000469 | 0.405378 | UBE1C |
| AF032456 | 0.001377 | 0.000525 | 5.89E−05 | 0.269833 | UBE2G2 |
| Y09723 | 0.00482 | 0.000525 | 0.000854 | −0.23772 | ZNF151 |
| AL031778 | 0.00482 | 0.000525 | 0.000239 | 0.178561 | |
| AL049218 | 0.00482 | 0.000525 | 0.001951 | 0.238837 | |
| AL080216 | 0.00482 | 0.000525 | 0.000349 | 0.311531 | |
| L00352 | 0.00482 | 0.000525 | 0.005084 | −0.39882 | |
| S79267 | 0.00482 | 0.000525 | 0.000908 | −0.19945 | |
| U94902 | 0.00482 | 0.000525 | 0.002556 | −0.17833 | |
| AA206524 | 0.000344 | 0.000567 | 0.000172 | 0.161868 | BART1 |
| AA926959 | 0.000344 | 0.000567 | 8.47E−05 | 0.169915 | CKS1 |
| M27543 | 0.000344 | 0.000567 | 0.000319 | −0.48924 | GNAI3 |
| AF019386 | 0.000344 | 0.000567 | 0.000399 | −0.18103 | HS3ST1 |
| AB006537 | 0.000344 | 0.000567 | 0.000658 | −0.1547 | IL1RAP |
| AJ001306 | 0.000344 | 0.000567 | 5.7E−05 | 0.338818 | INADL |
| AB011116 | 0.000344 | 0.000567 | 0.000355 | 0.25593 | KIAA0544 |
| AB029014 | 0.000344 | 0.000567 | 0.001352 | −0.1261 | KIAA1091 |
| AB029027 | 0.000344 | 0.000567 | 0.00209 | 0.180974 | KIAA1104 |
| M10901 | 0.000344 | 0.000567 | 4E−05 | −0.42455 | NR3C1 |
| D30036 | 0.000344 | 0.000567 | 5.62E−05 | −0.15539 | PITPN |
| U47077 | 0.000344 | 0.000567 | 0.001173 | 0.30799 | PRKDC |
| AF006751 | 0.000344 | 0.000567 | 3.4E−05 | −0.23462 | RRBP1 |
| AB006198 | 0.000344 | 0.000567 | 0.001465 | 0.256734 | SART1 |
| D63780 | 0.000344 | 0.000567 | 0.00021 | 0.374406 | STK25 |
| W28892 | 0.000344 | 0.000567 | 8.26E−05 | 0.803602 | SUI1 |
| M74524 | 0.000344 | 0.000567 | 0.000421 | −0.31531 | UBE2A |
| AL031230 | 0.000344 | 0.000567 | 6.83E−05 | 0.272378 | |
| AF057160 | 0.001377 | 0.000588 | 0.000279 | 0.307281 | ADPRTL1 |
| M74491 | 0.001377 | 0.000588 | 3.02E−05 | 0.170825 | ARF3 |
| AL120559 | 0.001377 | 0.000588 | 4.8E−05 | −0.64478 | ARPP-19 |
| D13630 | 0.001377 | 0.000588 | 3.01E−05 | −0.42457 | BZAP45 |
| U83246 | 0.001377 | 0.000588 | 0.003502 | 0.133045 | CPNE1 |
| AL050390 | 0.001377 | 0.000588 | 0.000139 | 0.231898 | DKFZP564O043 |
| D13315 | 0.001377 | 0.000588 | 0.000203 | 0.371377 | GLO1 |
| H12458 | 0.001377 | 0.000588 | 5.42E−05 | −0.22578 | H12458 yj12d03.s1 |
| AI347088 | 0.001377 | 0.000588 | 0.000151 | 0.321012 | HMG17L3 |
| X59770 | 0.001377 | 0.000588 | 0.001417 | −0.36292 | IL1R2 |
| AB007855 | 0.000344 | 0.000588 | 1.02E−05 | 0.086396 | KIAA0395 |
| AB016816 | 0.001377 | 0.000588 | 0.000751 | 0.146218 | MASL1 |
| U07132 | 0.001377 | 0.000588 | 0.002035 | −0.27336 | NR1H2 |
| AB019409 | 0.001377 | 0.000588 | 0.001479 | 0.154377 | PDL-108 |
| AB020641 | 0.001377 | 0.000588 | 0.00342 | 0.174568 | PFTK1 |
| AL050259 | 0.001377 | 0.000588 | 0.001864 | 0.272972 | RAB2L |
| AA099265 | 0.001377 | 0.000588 | 0.000614 | 0.38275 | RECK |
| X75042 | 0.001377 | 0.000588 | 6.85E−05 | −0.39572 | REL |
| AL050290 | 0.001377 | 0.000588 | 0.002426 | −0.28771 | SAT |
| AJ006417 | 0.001377 | 0.000588 | 0.000125 | −0.18595 | TBCD |
| X02812 | 0.001377 | 0.000588 | 1.78E−05 | −0.16423 | TGFB1 |
| AL050262 | 0.001377 | 0.000588 | 0.0031 | 0.348226 | TLR1 |
| X16576 | 0.001377 | 0.000588 | 9.49E−05 | 0.431692 | ZNF46 |
| X91249 | 0.000344 | 0.000609 | 1.04E−05 | −0.3925 | ABCG1 |
| Y00486 | 0.000344 | 0.000609 | 0.000297 | 0.259418 | APRT |
| U10473 | 0.000344 | 0.000609 | 0.000103 | −0.15424 | B4GALT1 |
| AB014595 | 0.000344 | 0.000609 | 5.19E−05 | 0.320955 | CUL4B |
| Y15227 | 0.000344 | 0.000609 | 4.08E−05 | 0.222481 | DLEU1 |
| U85267 | 0.000344 | 0.000609 | 0.000131 | 0.142894 | DSCR1 |
| AB019036 | 0.000344 | 0.000609 | 0.000336 | 0.177649 | GGPS1 |
| U90313 | 0.000344 | 0.000609 | 0.001838 | −0.25377 | GSTTLp28 |
| L42243 | 0.000344 | 0.000609 | 0.000201 | 0.403838 | HUMIFNAM08 |
| X16983 | 0.000344 | 0.000609 | 0.000317 | 0.232935 | ITGA4 |
| AB002368 | 0.000344 | 0.000609 | 0.001709 | 0.215217 | KIAA0370 |
| AI521453 | 0.000344 | 0.000609 | 0.000707 | −0.22735 | PC4 |
| Y08110 | 0.000344 | 0.000609 | 9.87E−05 | 0.260436 | SORL1 |
| D38122 | 0.000344 | 0.000609 | 6.27E−05 | −0.61781 | TNFSF6 |
| U49278 | 0.000344 | 0.000609 | 0.000173 | 0.204424 | UBE2V1 |
| X99050 | 0.000344 | 0.000609 | 7.72E−05 | 0.289751 | UVRAG |
| Z93930 | 0.000344 | 0.000609 | 0.00015 | −0.26558 | XBP1 |
| AF015767 | 0.000344 | 0.000659 | 0.000199 | 0.578977 | BRE |
| M34677 | 0.000344 | 0.000659 | 0.000647 | 0.198622 | F8A |
| J00210 | 0.000344 | 0.000659 | 0.002453 | −0.18828 | IFNA1 |
| AJ007583 | 0.000344 | 0.000659 | 0.00506 | −0.12644 | LARGE |
| M36881 | 0.000344 | 0.000659 | 0.000302 | 0.328248 | LCK |
| X70326 | 0.000344 | 0.000659 | 0.000132 | −0.58974 | MACMARCKS |
| M64571 | 0.000344 | 0.000659 | 0.000158 | 0.157573 | MAP4 |
| AI345944 | 0.000344 | 0.000659 | 0.000363 | 0.311507 | NDUFB1 |
| D23662 | 0.000344 | 0.000659 | 0.000171 | 0.289452 | NEDD8 |
| M14630 | 0.000344 | 0.000659 | 1.26E−05 | −0.1626 | PTMA |
| D64015 | 0.000344 | 0.000659 | 0.001012 | 0.195679 | TIAL1 |
| M63582 | 0.000344 | 0.000659 | 2.66E−05 | −0.39175 | |
| U79300 | 0.000344 | 0.000659 | 0.000196 | −0.16218 | |
| D29805 | 0.00482 | 0.000812 | 0.000289 | −0.23044 | B4GALT1 |
| U47414 | 0.001377 | 0.000812 | 0.000137 | 0.262974 | CCNG2 |
| L33930 | 0.001377 | 0.000812 | 5.56E−06 | 0.343203 | CD24 |
| AL050164 | 0.00482 | 0.000812 | 0.000345 | 0.307729 | CDYL |
| D10040 | 0.001377 | 0.000812 | 1.49E−05 | −0.45708 | FACL2 |
| M36820 | 0.00482 | 0.000812 | 7.67E−05 | −0.49075 | GRO2 |
| U77948 | 0.00482 | 0.000812 | 0.000511 | 0.286776 | GTF2I |
| X56681 | 0.00482 | 0.000812 | 0.000503 | −0.18359 | JUND |
| AF070569 | 0.00482 | 0.000812 | 0.000446 | −0.6104 | MGC14376 |
| W28205 | 0.00482 | 0.000812 | 0.00017 | −0.21741 | MKLN1 |
| U61981 | 0.001377 | 0.000812 | 0.000725 | 0.203996 | MSH3 |
| AB014547 | 0.001377 | 0.000812 | 7.73E−05 | 0.217806 | MTMR4 |
| AL050366 | 0.00482 | 0.000812 | 0.001126 | 0.421541 | OGT |
| U89606 | 0.001377 | 0.000812 | 6.13E−05 | −0.19512 | PDXK |
| D10495 | 0.00482 | 0.000812 | 0.000433 | 0.290156 | PRKCD |
| D42063 | 0.001377 | 0.000812 | 0.000346 | −0.52828 | RANBP2 |
| H68340 | 0.00482 | 0.000812 | 0.004081 | −0.3419 | RNAHP |
| AF059617 | 0.001377 | 0.000812 | 0.00012 | −0.27807 | SNK |
| AB028950 | 0.00482 | 0.000812 | 0.000365 | 0.313606 | TLN1 |
| L41690 | 0.001377 | 0.000812 | 0.000109 | 0.401776 | TRADD |
| X95384 | 0.00482 | 0.000812 | 0.00053 | 0.327055 | UK114 |
| X98054 | 0.00482 | 0.001094 | 4.71E−05 | −0.12615 | CREBL1 |
| J05036 | 0.00482 | 0.001094 | 0.00171 | 0.064463 | CTSE |
| AF001434 | 0.00482 | 0.001094 | 0.000161 | −0.26223 | EHD1 |
| L18960 | 0.00482 | 0.001094 | 3.26E−05 | −0.38369 | EIF1A |
| AB014555 | 0.00482 | 0.001094 | 0.001608 | −0.18202 | KIAA0655 |
| X76057 | 0.00482 | 0.001094 | 0.000352 | 0.193745 | MPI |
| X74594 | 0.00482 | 0.001094 | 0.000352 | 0.439326 | RBL2 |
| AF044309 | 0.00482 | 0.001094 | 0.000217 | −0.2163 | STX11 |
| U07158 | 0.00482 | 0.001094 | 0.000122 | −0.2301 | STX4A |
| L40386 | 0.00482 | 0.001094 | 7.97E−05 | −0.19863 | TFDP2 |
| H97470 | 0.00482 | 0.001094 | 0.000624 | −0.10587 | |
| U78027 | 0.00482 | 0.001094 | 0.000804 | 0.340784 | |
| U50534 | 0.001377 | 0.001345 | 0.00039 | 0.250627 | 13CDNA73 |
| X55330 | 0.001377 | 0.001345 | 9.3E−05 | 0.493025 | AGA |
| L19605 | 0.001377 | 0.001345 | 0.004442 | 0.183134 | ANXA11 |
| Y00097 | 0.001377 | 0.001345 | 4.42E−05 | 0.409932 | ANXA6 |
| U26455 | 0.00482 | 0.001345 | 0.000705 | 0.499049 | ATM |
| AF047473 | 0.001377 | 0.001345 | 5.14E−05 | 0.226 | BUB3 |
| M95724 | 0.00482 | 0.001345 | 0.002166 | −0.46553 | CENPC1 |
| AB014558 | 0.001377 | 0.001345 | 0.004662 | −0.44793 | CRY2 |
| R38263 | 0.001377 | 0.001345 | 0.00048 | −0.12843 | DJ347H13.4 |
| AI434146 | 0.001377 | 0.001345 | 0.000397 | 0.187485 | DKFZp570I0164 |
| D12686 | 0.00482 | 0.001345 | 0.008744 | −0.11456 | EIF4G1 |
| AF059611 | 0.00482 | 0.001345 | 0.000694 | −0.27343 | ENC1 |
| X59834 | 0.001377 | 0.001345 | 0.004986 | −0.34836 | GLUL |
| D64142 | 0.001377 | 0.001345 | 0.000169 | 0.293999 | H1FX |
| U51333 | 0.001377 | 0.001345 | 0.000376 | 0.273402 | HK3 |
| M59488 | 0.001377 | 0.001345 | 0.001357 | −0.13313 | HUMS100B3 |
| X58529 | 0.001377 | 0.001345 | 0.000417 | 1.04789 | IGHM |
| D79983 | 0.001377 | 0.001345 | 2.97E−05 | 0.387491 | KIAA0161 |
| AB002370 | 0.00482 | 0.001345 | 0.00052 | 0.425557 | KIAA0372 |
| AB007863 | 0.001377 | 0.001345 | 0.000128 | 0.29668 | KIAA0403 |
| AB014549 | 0.001377 | 0.001345 | 0.001505 | 0.42387 | KIAA0649 |
| AB020711 | 0.00482 | 0.001345 | 0.002079 | 0.222346 | KIAA0904 |
| AB002357 | 0.001377 | 0.001345 | 0.001045 | 0.317849 | KIF3B |
| U09284 | 0.00482 | 0.001345 | 0.000792 | −0.23635 | LIMS1 |
| D50810 | 0.001377 | 0.001345 | 5.17E−05 | −0.1859 | LNPEP |
| U18259 | 0.001377 | 0.001345 | 0.000153 | 0.229322 | MHC2TA |
| AF041080 | 0.00482 | 0.001345 | 0.001964 | 0.367098 | MN7 |
| X70991 | 0.001377 | 0.001345 | 0.00203 | −0.14032 | NAB2 |
| AC002045 | 0.00482 | 0.001345 | 0.00028 | 0.326033 | NPIP |
| U92538 | 0.001377 | 0.001345 | 0.00149 | 0.2372 | ORC5L |
| U24153 | 0.001377 | 0.001345 | 0.00018 | −0.36291 | PAK2 |
| Z49194 | 0.001377 | 0.001345 | 0.000519 | 0.215733 | POU2AF1 |
| AF016371 | 0.001377 | 0.001345 | 0.001059 | 0.240562 | PPIH |
| AF020736 | 0.001377 | 0.001345 | 6.26E−05 | −0.32893 | PSMC4 |
| D11327 | 0.001377 | 0.001345 | 0.00019 | −0.74969 | PTPN7 |
| AF098799 | 0.00482 | 0.001345 | 0.001893 | −0.3646 | RANBP7 |
| M22995 | 0.001377 | 0.001345 | 0.005586 | 0.270032 | RAP1A |
| L11566 | 0.001377 | 0.001345 | 0.000291 | 0.17032 | RPL18 |
| U71364 | 0.001377 | 0.001345 | 0.000276 | −0.24064 | SERPINB9 |
| X07834 | 0.00482 | 0.001345 | 0.000362 | −0.21917 | SOD2 |
| X05839 | 0.001377 | 0.001345 | 0.000779 | −0.20819 | TGFB1 |
| AB000509 | 0.001377 | 0.001345 | 3.63E−05 | 0.460686 | TRAF5 |
| U82130 | 0.001377 | 0.001345 | 4.69E−05 | −0.36064 | TSG101 |
| L16842 | 0.001377 | 0.001345 | 0.001533 | 0.189597 | UQCRC1 |
| X51521 | 0.001377 | 0.001345 | 0.000379 | −0.62845 | VIL2 |
| M86400 | 0.001377 | 0.001345 | 0.000132 | −0.30595 | YWHAZ |
| AF041259 | 0.001377 | 0.001345 | 0.001393 | 0.202001 | ZNF217 |
| AA977136 | 0.001377 | 0.001345 | 0.001953 | 0.095364 | |
| AI624038 | 0.001377 | 0.001345 | 0.001833 | −0.16137 | |
| AL050148 | 0.00482 | 0.001345 | 0.000905 | 0.266795 | |
| HG2709-HT2805 | 0.001377 | 0.001345 | 0.000134 | −0.22645 | |
| HG3227-HT3404 | 0.001377 | 0.001345 | 4.02E−05 | −0.23244 | |
| M28225 | 0.00482 | 0.001345 | 0.001444 | −0.95152 | |
| U80017 | 0.001377 | 0.001345 | 0.004917 | 0.171432 | |
| X55544 | 0.001377 | 0.001467 | 0.001049 | −0.12406 | ATF1 |
| X52560 | 0.001377 | 0.001467 | 8.17E−05 | −0.50375 | CEBPB |
| AA044787 | 0.001377 | 0.001467 | 0.001147 | 0.289086 | CNOT8 |
| AF017790 | 0.001377 | 0.001467 | 6.88E−06 | 0.382661 | HEC |
| D00749 | 0.001377 | 0.001467 | 0.000131 | −0.10539 | HUMCD7G3 |
| AB007890 | 0.001377 | 0.001467 | 0.000838 | 0.200677 | KIAA0430 |
| L35251 | 0.001377 | 0.001467 | 0.000873 | 0.12909 | MFAP3 |
| AF098638 | 0.001377 | 0.001467 | 0.000684 | −0.18761 | RAB5EP |
| AB004857 | 0.001377 | 0.001467 | 0.000471 | 0.23048 | SLC11A2 |
| U53347 | 0.001377 | 0.001467 | 0.001367 | −0.13658 | SLC1A5 |
| U04847 | 0.001377 | 0.001467 | 0.000403 | 0.117176 | SMARCB1 |
| M92843 | 0.001377 | 0.001467 | 3.3E−05 | −1.37866 | ZFP36 |
| AF033199 | 0.001377 | 0.001467 | 0.00019 | 0.237743 | ZNF204 |
| AC004893 | 0.001377 | 0.001467 | 0.000617 | −0.25759 | |
| AL050151 | 0.001377 | 0.001467 | 8.03E−06 | −0.80887 | |
| U80770 | 0.001377 | 0.001467 | 0.006738 | −0.12644 | |
| W27675 | 0.00482 | 0.001614 | 0.005157 | 0.468709 | CDA02 |
| AI056696 | 0.00482 | 0.001614 | 0.000665 | 0.215941 | CETN3 |
| AF062536 | 0.00482 | 0.001614 | 0.005001 | 0.197482 | CUL1 |
| D29643 | 0.00482 | 0.001614 | 0.0005 | 0.157183 | DDOST |
| AA181196 | 0.00482 | 0.001614 | 0.000166 | 0.119162 | FLJ11712 |
| W07033 | 0.001377 | 0.001614 | 0.000136 | 0.347648 | GMFG |
| Z18859 | 0.00482 | 0.001614 | 0.000684 | 0.181514 | GNAT2 |
| U83660 | 0.00482 | 0.001614 | 0.00114 | 0.136411 | HSU83660 |
| AA628946 | 0.00482 | 0.001614 | 0.002684 | 0.337197 | KHSRP |
| D13626 | 0.00482 | 0.001614 | 0.005837 | 0.254138 | KIAA0001 |
| AB002340 | 0.00482 | 0.001614 | 0.002977 | 0.168464 | KIAA0342 |
| AB002353 | 0.001377 | 0.001614 | 0.000119 | 0.305921 | KIAA0355 |
| U32849 | 0.00482 | 0.001614 | 0.000272 | 0.345048 | NMI |
| S79219 | 0.00482 | 0.001614 | 0.000119 | 0.167463 | PCCA |
| L37127 | 0.00482 | 0.001614 | 0.010173 | 0.103446 | POLR2J |
| M35416 | 0.00482 | 0.001614 | 0.001433 | 0.33505 | RALB |
| X76061 | 0.00482 | 0.001614 | 0.000273 | 0.378113 | RBL2 |
| AF061741 | 0.00482 | 0.001614 | 0.004586 | 0.221278 | SDR1 |
| D31891 | 0.001377 | 0.001614 | 0.000819 | 0.161458 | SETDB1 |
| W26406 | 0.00482 | 0.001614 | 0.000479 | 0.300512 | SIAH1 |
| X84002 | 0.00482 | 0.001614 | 0.000699 | 0.143479 | TAF2J |
| U81006 | 0.00482 | 0.001614 | 0.003151 | 0.255479 | TM9SF2 |
| U69108 | 0.00482 | 0.001614 | 0.000657 | 0.208286 | TRAF5 |
| S66666 | 0.00482 | 0.001614 | 0.002417 | 0.119478 | |
| U84388 | 0.00482 | 0.001719 | 0.000197 | −0.18606 | CRADD |
| L08069 | 0.00482 | 0.001719 | 0.001985 | −0.31866 | DNAJA1 |
| U41514 | 0.00482 | 0.001719 | 9.38E−05 | −0.44803 | GALNT1 |
| M69013 | 0.001377 | 0.001719 | 6.26E−05 | −0.1948 | GNA11 |
| L11706 | 0.00482 | 0.001719 | 0.001422 | −0.16675 | LIPE |
| R92331 | 0.00482 | 0.001719 | 0.000198 | −0.24196 | MT1E |
| X64318 | 0.00482 | 0.001719 | 0.006253 | −0.37391 | NFIL3 |
| X12458 | 0.00482 | 0.001719 | 0.001489 | −0.33668 | P3 |
| M25393 | 0.00482 | 0.001719 | 0.000958 | −0.23304 | PTPN2 |
| M59465 | 0.00482 | 0.001719 | 0.0002 | −0.96074 | TNFAIP3 |
| AF084260 | 0.00482 | 0.001719 | 0.001063 | −0.39491 | TRIP15 |
| HG2149-HT2219 | 0.00482 | 0.001719 | 0.001437 | −0.14432 | |
| AB021663 | 0.00482 | 0.001963 | 0.00027 | −0.13923 | ATF5 |
| AL080209 | 0.00482 | 0.001963 | 0.000337 | 0.437957 | DKFZP586F2423 |
| M34641 | 0.00482 | 0.001963 | 0.002352 | −0.14552 | FGFR1 |
| AL096714 | 0.001377 | 0.001963 | 0.000469 | 0.224782 | FLJ20113 |
| AB011124 | 0.001377 | 0.001963 | 0.000101 | −0.17709 | KIAA0552 |
| AB020633 | 0.001377 | 0.001963 | 0.00082 | 0.308616 | KIAA0826 |
| AB029020 | 0.001377 | 0.001963 | 0.00061 | 0.3824 | KIAA1097 |
| X76220 | 0.001377 | 0.001963 | 7.21E−05 | 0.444366 | MAL |
| AF040964 | 0.00482 | 0.001963 | 0.001161 | −0.54746 | MGC4701 |
| U91512 | 0.001377 | 0.001963 | 0.00035 | −0.55826 | NINJ1 |
| U60325 | 0.00482 | 0.001963 | 0.000288 | −0.14386 | POLG |
| Z15108 | 0.001377 | 0.001963 | 0.000107 | 0.176424 | PRKCZ |
| Y08262 | 0.001377 | 0.001963 | 0.000183 | 0.377974 | SCA2 |
| U30246 | 0.001377 | 0.001963 | 0.000209 | −0.25952 | SLC12A2 |
| J04137 | 0.001377 | 0.001963 | 0.000641 | −0.22175 | SSA2 |
| M38449 | 0.001377 | 0.001963 | 0.0004 | −0.29059 | TGFB1 |
| AC005757 | 0.00482 | 0.001963 | 0.000169 | 0.387439 | |
| HG825-HT825 | 0.001377 | 0.001963 | 0.000553 | −0.19964 | |
| AF047348 | 0.001377 | 0.002207 | 0.000292 | 0.202669 | APBA2 |
| AF053977 | 0.001377 | 0.002207 | 0.003143 | 0.134266 | CDC23 |
| AF083322 | 0.001377 | 0.002207 | 0.000344 | 0.272282 | CEP1 |
| AL050369 | 0.001377 | 0.002207 | 0.001224 | 0.241992 | DKFZP566J153 |
| D32257 | 0.001377 | 0.002207 | 0.000238 | 0.300058 | GTF3A |
| M65217 | 0.001377 | 0.002207 | 0.000232 | 0.249614 | HSF2 |
| AB014574 | 0.001377 | 0.002207 | 0.000606 | 0.130056 | KIAA0674 |
| AB029023 | 0.001377 | 0.002207 | 0.000219 | 0.219428 | KIAA1100 |
| Z34975 | 0.001377 | 0.002207 | 8.88E−05 | 0.41432 | LDLC |
| D83597 | 0.001377 | 0.002207 | 0.000136 | 0.249838 | LY64 |
| U09759 | 0.001377 | 0.002207 | 0.000842 | 0.330751 | MAPK9 |
| U59302 | 0.001377 | 0.002207 | 0.000241 | 0.309348 | NCOA1 |
| AJ005698 | 0.001377 | 0.002207 | 0.004173 | 0.139618 | PARN |
| X54871 | 0.001377 | 0.002207 | 0.010035 | 0.119258 | RAB5B |
| AL080198 | 0.001377 | 0.002207 | 0.002866 | 0.251598 | RENT2 |
| M74447 | 0.001377 | 0.002207 | 0.000444 | 0.093537 | TAP2 |
| J04973 | 0.001377 | 0.002207 | 0.011696 | 0.141705 | UQCRC2 |
| U90902 | 0.001377 | 0.002207 | 0.001336 | 0.246217 | |
| U94333 | 0.001377 | 0.002323 | 0.004818 | −0.13898 | C1QR |
| U60808 | 0.001377 | 0.002323 | 0.000374 | −0.12217 | CDS1 |
| L08069 | 0.001377 | 0.002323 | 0.002161 | −0.29982 | DNAJA1 |
| AA552140 | 0.001377 | 0.002323 | 0.003368 | −0.22604 | E2F4 |
| M31210 | 0.001377 | 0.002323 | 0.000124 | −0.33555 | EDG1 |
| AI189287 | 0.001377 | 0.002323 | 0.002445 | −0.24115 | H1F2 |
| W25934 | 0.001377 | 0.002323 | 0.003385 | −0.32382 | JTV1 |
| Z98046 | 0.001377 | 0.002323 | 0.000122 | −0.33551 | MAGED2 |
| L76571 | 0.001377 | 0.002323 | 0.009985 | −0.12617 | NR0B2 |
| AF071504 | 0.001377 | 0.002323 | 0.000191 | −0.14267 | STX11 |
| X56687 | 0.001377 | 0.002323 | 0.00011 | −0.29728 | UBTF |
| AI097085 | 0.001377 | 0.002323 | 0.000941 | −0.16209 | |
| AA114830 | 0.001377 | 0.002503 | 0.000293 | 0.272601 | AKAP10 |
| AI991631 | 0.001377 | 0.002503 | 0.000132 | −0.11786 | BRD4 |
| U04343 | 0.001377 | 0.002503 | 4.45E−05 | −0.25478 | CD86 |
| M12824 | 0.001377 | 0.002503 | 0.008271 | −0.34597 | CD8A |
| U89896 | 0.001377 | 0.002503 | 0.006997 | −0.2182 | CSNK1G2 |
| AI432401 | 0.001377 | 0.002503 | 0.006072 | 0.32631 | FGL2 |
| AA176780 | 0.001377 | 0.002503 | 0.001296 | 0.14235 | HSA249128 |
| M21188 | 0.001377 | 0.002503 | 0.000165 | 0.251899 | IDE |
| U43572 | 0.001377 | 0.002503 | 0.000128 | 0.318327 | NAGLU |
| X02751 | 0.001377 | 0.002503 | 0.000359 | −0.2229 | NRAS |
| AF069250 | 0.001377 | 0.002503 | 0.00135 | 0.476217 | OA48-18 |
| D25328 | 0.001377 | 0.002503 | 0.000171 | 0.125335 | PFKP |
| AF010312 | 0.001377 | 0.002503 | 0.001216 | −0.47628 | PIG7 |
| M34668 | 0.001377 | 0.002503 | 0.000421 | 0.181315 | PTPRA |
| AF061836 | 0.001377 | 0.002503 | 0.001026 | 0.21847 | RASSF1 |
| AI535653 | 0.001377 | 0.002503 | 0.001712 | 0.34571 | SC4MOL |
| X75755 | 0.001377 | 0.002503 | 0.003813 | −0.2236 | SFRS2 |
| W16505 | 0.001377 | 0.002503 | 0.001699 | 0.101763 | SNRPD2 |
| L31529 | 0.001377 | 0.002503 | 0.000325 | 0.144265 | SNTB1 |
| D86970 | 0.001377 | 0.002503 | 0.000219 | 0.218777 | TIAF1 |
| AL050223 | 0.001377 | 0.002503 | 0.002632 | 0.2458 | VAMP2 |
| AA877215 | 0.001377 | 0.002503 | 0.008439 | −0.17878 | |
| AL049435 | 0.001377 | 0.002503 | 0.000111 | 0.194323 | |
| M76180 | 0.001377 | 0.002575 | 0.000698 | 0.162775 | DDC |
| M94065 | 0.001377 | 0.002575 | 0.000426 | 0.156894 | DHODH |
| J04988 | 0.001377 | 0.002575 | 6.17E−05 | −0.22304 | HSPCB |
| Z68907 | 0.001377 | 0.002575 | 0.000305 | 0.391111 | IDH3G |
| J03909 | 0.001377 | 0.002575 | 0.000837 | −0.35709 | IFI30 |
| AB011104 | 0.001377 | 0.002575 | 0.000729 | 0.227798 | KIAA0532 |
| AB011173 | 0.001377 | 0.002575 | 0.000585 | 0.283714 | KIAA0601 |
| U70322 | 0.001377 | 0.002575 | 0.000177 | −0.41259 | KPNB2 |
| D86961 | 0.001377 | 0.002575 | 0.001925 | −0.19403 | LHFPL2 |
| AF052111 | 0.001377 | 0.002575 | 0.000738 | 0.249468 | LOC51172 |
| AJ224875 | 0.001377 | 0.002575 | 0.005091 | 0.139606 | MGC2840 |
| M21985 | 0.001377 | 0.002575 | 0.001409 | −0.102 | NR2C1 |
| J05448 | 0.001377 | 0.002575 | 0.004982 | −0.15329 | POLR2C |
| AB006572 | 0.001377 | 0.002575 | 0.000169 | 0.213636 | RMP |
| AJ011712 | 0.001377 | 0.002575 | 0.011372 | 0.066711 | TNNT1 |
| AJ006973 | 0.001377 | 0.002575 | 0.000101 | −0.31773 | TOM1 |
| U67122 | 0.001377 | 0.002575 | 0.000364 | −0.14274 | UBL1 |
| U71598 | 0.001377 | 0.002575 | 0.003508 | 0.128607 | ZNF274 |
| M81118 | 0.001377 | 0.002575 | 0.00023 | 0.333526 | |
| U61166 | 0.001377 | 0.002575 | 0.003055 | −0.14488 | |
| U94902 | 0.001377 | 0.002575 | 0.000137 | −0.23298 | |
| Z82244 | 0.001377 | 0.002575 | 0.000258 | −0.53938 | |
| M36341 | 0.001377 | 0.002788 | 0.00056 | −0.38498 | ARF4 |
| L09159 | 0.001377 | 0.002788 | 0.00112 | 0.474985 | ARHA |
| U68485 | 0.00482 | 0.002788 | 0.006797 | 0.224774 | BIN1 |
| Z22555 | 0.001377 | 0.002788 | 0.005455 | −0.16351 | CD36L1 |
| D44497 | 0.00482 | 0.002788 | 0.004453 | 0.131654 | CORO1A |
| L37042 | 0.00482 | 0.002788 | 0.000582 | −0.33273 | CSNK1A1 |
| M74099 | 0.001377 | 0.002788 | 0.00014 | 0.389638 | CUTL1 |
| AL080159 | 0.001377 | 0.002788 | 0.002335 | −0.11101 | DKFZP434M154 |
| AF004292 | 0.001377 | 0.002788 | 0.001306 | −0.2375 | DKFZP566C134 |
| AF088982 | 0.001377 | 0.002788 | 0.001264 | −0.22098 | DNAJB5 |
| U73704 | 0.001377 | 0.002788 | 0.001412 | −0.17508 | FAP48 |
| M77810 | 0.00482 | 0.002788 | 0.000209 | −0.15854 | GATA2 |
| U67369 | 0.00482 | 0.002788 | 0.00347 | 0.137095 | GFI1 |
| D00632 | 0.001377 | 0.002788 | 0.001138 | −0.15517 | GPX3 |
| X99270 | 0.001377 | 0.002788 | 0.00065 | 0.191612 | HSXQ28ORF |
| D42041 | 0.00482 | 0.002788 | 0.003705 | 0.195279 | KIAA0088 |
| AA524058 | 0.001377 | 0.002788 | 0.000288 | 0.360599 | LOC51020 |
| U77604 | 0.001377 | 0.002788 | 0.00233 | 0.296247 | MGST2 |
| J04031 | 0.00482 | 0.002788 | 0.000148 | 0.290038 | MTHFD1 |
| AF025794 | 0.001377 | 0.002788 | 0.006763 | 0.107466 | MTRR |
| D86326 | 0.001377 | 0.002788 | 0.008217 | 0.124987 | P115 |
| U14417 | 0.001377 | 0.002788 | 0.000521 | −0.13999 | RALGDS |
| U85611 | 0.001377 | 0.002788 | 0.000126 | −0.3879 | SIP2-28 |
| U66617 | 0.001377 | 0.002788 | 0.001462 | −0.14653 | SMARCD1 |
| X59960 | 0.001377 | 0.002788 | 0.005127 | −0.11069 | SMPD1 |
| AF031166 | 0.001377 | 0.002788 | 0.001219 | 0.110457 | SRP46 |
| U86136 | 0.001377 | 0.002788 | 0.000542 | 0.149235 | TEP1 |
| U16296 | 0.001377 | 0.002788 | 0.00138 | 0.136848 | TIAM1 |
| D50917 | 0.001377 | 0.002788 | 0.000467 | 0.402091 | TRIP-Br2 |
| AC004770 | 0.001377 | 0.002788 | 0.001042 | −0.10615 | |
| J03071 | 0.001377 | 0.002788 | 0.0116 | 0.17732 | |
| D67031 | 0.00482 | 0.004163 | 0.000204 | 0.543743 | ADD3 |
| U68030 | 0.00482 | 0.004163 | 0.000279 | −0.16075 | CCR6 |
| U41387 | 0.00482 | 0.004163 | 8.67E−05 | −0.29576 | DDX21 |
| AF084535 | 0.00482 | 0.004163 | 0.002306 | 0.159095 | EPM2A |
| AI417075 | 0.00482 | 0.004163 | 0.000538 | 0.330385 | FLJ14040 |
| D82348 | 0.00482 | 0.004349 | 0.006063 | 0.245422 | ATIC |
| AA648295 | 0.00482 | 0.004349 | 0.002422 | 0.337484 | CBX3 |
| U79270 | 0.00482 | 0.004349 | 0.001345 | 0.460807 | COX11 |
| AF071748 | 0.00482 | 0.004349 | 0.002471 | 0.170315 | CTSF |
| AL080088 | 0.00482 | 0.004349 | 0.000207 | 0.165357 | DKFZP564K2062 |
| AI540318 | 0.00482 | 0.004349 | 0.00055 | −0.13789 | DNAJB6 |
| U03272 | 0.00482 | 0.004349 | 0.003893 | 0.101031 | FBN2 |
| Z97989 | 0.00482 | 0.004349 | 0.001126 | −0.41969 | FYN |
| AF042379 | 0.00482 | 0.004349 | 0.008308 | 0.176604 | GCP2 |
| U73737 | 0.00482 | 0.004349 | 0.00262 | −0.14396 | HUMMSH06 |
| AF031167 | 0.00482 | 0.004349 | 0.000117 | 0.201914 | IL15 |
| D83778 | 0.00482 | 0.004349 | 0.000504 | −0.2149 | KIAA0194 |
| AB028965 | 0.00482 | 0.004349 | 0.007277 | 0.125446 | KIAA1042 |
| M79321 | 0.00482 | 0.004349 | 0.003247 | −0.21992 | LYN |
| L11284 | 0.00482 | 0.004349 | 0.003994 | −0.09508 | MAP2K1 |
| AJ000882 | 0.00482 | 0.004349 | 0.00042 | 0.180413 | NCOA1 |
| L41067 | 0.00482 | 0.004349 | 0.000388 | 0.370635 | NFATC3 |
| AF057297 | 0.00482 | 0.004349 | 0.001191 | 0.521103 | OAZ2 |
| X66360 | 0.00482 | 0.004349 | 0.001123 | −0.17473 | PCTK2 |
| U24183 | 0.00482 | 0.004349 | 0.001156 | 0.141168 | PFKM |
| L42373 | 0.00482 | 0.004349 | 0.006706 | 0.165885 | PPP2R5A |
| AB018288 | 0.00482 | 0.004349 | 0.003777 | 0.184227 | RANBP16 |
| M58459 | 0.00482 | 0.004349 | 0.008923 | −1.04752 | RPS4Y |
| M60725 | 0.00482 | 0.004349 | 0.001349 | −0.10092 | RPS6KB1 |
| Y10931 | 0.00482 | 0.004349 | 0.001246 | 0.194156 | SPK |
| AB004904 | 0.00482 | 0.004349 | 0.000233 | −0.31373 | SSI-3 |
| AF060798 | 0.00482 | 0.004349 | 0.000809 | 0.142845 | STK16 |
| U66867 | 0.00482 | 0.004349 | 0.011318 | 0.150812 | UBE2I |
| AB028980 | 0.00482 | 0.004349 | 0.001471 | 0.265042 | USP24 |
| AF052107 | 0.00482 | 0.004349 | 0.002884 | 0.197902 | |
| AL031985 | 0.00482 | 0.004349 | 0.000181 | −0.24089 | |
| D26121 | 0.00482 | 0.004349 | 0.00387 | −0.16268 | |
| W28667 | 0.00482 | 0.004349 | 0.004391 | 0.476395 | |
| AL050157 | 0.00482 | 0.004467 | 0.000289 | 0.269949 | DKFZPS86O0120 |
| U31930 | 0.00482 | 0.004467 | 0.000244 | 0.349997 | DUT |
| AI951946 | 0.00482 | 0.004467 | 8.71E−05 | 0.401112 | HBOA |
| AB002354 | 0.00482 | 0.004467 | 0.001517 | −0.13368 | KIAA0356 |
| M36067 | 0.00482 | 0.004467 | 8.15E−05 | 0.277858 | LIG1 |
| J02783 | 0.00482 | 0.004467 | 0.002151 | −0.21979 | P4HB |
| M37238 | 0.00482 | 0.004467 | 0.005333 | −0.15474 | PLCG2 |
| M99438 | 0.00482 | 0.004467 | 6.2E−05 | −0.36844 | TLE3 |
| Z97630 | 0.00482 | 0.004467 | 0.002794 | 0.217849 | |
| D14874 | 0.00482 | 0.005608 | 0.000611 | −0.55358 | ADM |
| L08177 | 0.00482 | 0.005608 | 0.000434 | −0.49252 | EBI2 |
| U09510 | 0.00482 | 0.005608 | 5.27E−05 | −0.57567 | GARS |
| L05424 | 0.00482 | 0.005608 | 0.000114 | −0.39048 | HUMSCG19 |
| X13956 | 0.00482 | 0.005608 | 0.004098 | 0.187622 | MGC10471 |
| U88620 | 0.00482 | 0.005608 | 0.00074 | 0.345628 | OGG1 |
| M29551 | 0.00482 | 0.005608 | 0.000471 | 0.319301 | PPP3CB |
| AF068836 | 0.00482 | 0.005608 | 0.000323 | −0.23628 | PSCDBP |
| U08316 | 0.00482 | 0.005608 | 0.0006 | 0.205899 | RPS6KA3 |
| J02966 | 0.00482 | 0.005608 | 0.000498 | −0.11291 | SLC25A4 |
| AF107463 | 0.00482 | 0.005608 | 0.002824 | −0.36924 | SPF30 |
| AB000450 | 0.00482 | 0.005608 | 0.000256 | −0.24717 | VRK2 |
| AF070590 | 0.00482 | 0.005608 | 0.000834 | 0.127523 | |
| AF001383 | 0.00482 | 0.006065 | 0.002075 | 0.16512 | BIN1 |
| AF026291 | 0.00482 | 0.006065 | 0.000385 | −0.16859 | CCT4 |
| D63877 | 0.00482 | 0.006065 | 0.005226 | −0.13956 | KIAA0157 |
| U14383 | 0.00482 | 0.006065 | 0.002623 | −0.18669 | MUC8 |
| U68140 | 0.00482 | 0.006065 | 0.000677 | 0.172443 | NVL |
| L25441 | 0.00482 | 0.006065 | 0.000613 | −0.17207 | PGGT1B |
| U46751 | 0.00482 | 0.006065 | 0.000578 | −0.38675 | SQSTM1 |
| HG4740-HT5187 | 0.00482 | 0.006065 | 0.00867 | 0.146562 | |
| W26851 | 0.00482 | 0.006065 | 0.002052 | 0.312992 | |
| U78735 | 0.00482 | 0.006347 | 0.000529 | −0.09197 | ABCA3 |
| Y12226 | 0.00482 | 0.006347 | 0.000397 | −0.21303 | AP1G1 |
| D38293 | 0.00482 | 0.006347 | 0.002894 | −0.24373 | AP3M2 |
| X14046 | 0.00482 | 0.006347 | 0.001084 | 0.134786 | CD37 |
| AF026004 | 0.00482 | 0.006347 | 0.008622 | −0.07494 | CLCN2 |
| U46023 | 0.00482 | 0.006347 | 0.000273 | −0.17969 | CXorf6 |
| AL080178 | 0.00482 | 0.006347 | 0.000683 | 0.260343 | DKFZP434K171 |
| AL080118 | 0.00482 | 0.006347 | 0.001904 | −0.28696 | DKFZP564F1123 |
| AL050197 | 0.00482 | 0.006347 | 0.004294 | 0.233045 | DKFZP586D0623 |
| X68277 | 0.00482 | 0.006347 | 0.011411 | −0.42385 | DUSP1 |
| X03674 | 0.00482 | 0.006347 | 0.008478 | 0.174463 | G6PD |
| Y13286 | 0.00482 | 0.006347 | 0.004068 | 0.134985 | GDI2 |
| U19247 | 0.00482 | 0.006347 | 0.000589 | −0.29688 | HSINFGRA7 |
| AB023163 | 0.00482 | 0.006347 | 0.002537 | 0.194491 | HYPH |
| L36818 | 0.00482 | 0.006347 | 0.007182 | 0.204818 | INPPL1 |
| U51127 | 0.00482 | 0.006347 | 0.003952 | 0.108702 | IRF5 |
| M15395 | 0.00482 | 0.006347 | 0.001863 | 0.402323 | ITGB2 |
| U51336 | 0.00482 | 0.006347 | 0.008615 | 0.336527 | ITPK1 |
| AJ000008 | 0.00482 | 0.006347 | 0.000256 | −0.14181 | PIK3C2G |
| AI126004 | 0.00482 | 0.006347 | 0.000954 | 0.262925 | SAS10 |
| AF051325 | 0.00482 | 0.006347 | 0.000144 | −0.43952 | SH2D2A |
| U79528 | 0.00482 | 0.006347 | 0.002518 | 0.158101 | SR-BP1 |
| U52426 | 0.00482 | 0.006347 | 9.31E−05 | 0.411984 | STIM1 |
| AB018339 | 0.00482 | 0.006347 | 0.000751 | 0.199758 | SYNE-1B |
| D43642 | 0.00482 | 0.006347 | 0.0005 | 0.305805 | TCFL1 |
| D29767 | 0.00482 | 0.006347 | 0.003934 | −0.09702 | TEC |
| M92383 | 0.00482 | 0.006347 | 0.001466 | 0.219769 | TMSB10 |
| AA192359 | 0.00482 | 0.006347 | 0.00028 | 0.17619 | TRN-SR |
| AC004472 | 0.00482 | 0.006347 | 0.002169 | −0.15115 | |
| AF052138 | 0.00482 | 0.006347 | 0.000189 | 0.441167 | |
| X15674 | 0.00482 | 0.006347 | 0.007899 | −0.10738 | |
| Z82215 | 0.00482 | 0.006347 | 0.002527 | 0.153792 | |
| AF070523 | 0.00482 | 0.006634 | 0.00037 | 0.437983 | JWA |
| D13641 | 0.00482 | 0.006634 | 0.000776 | 0.275308 | KIAA0016 |
| X79204 | 0.00482 | 0.006634 | 0.000182 | 0.256049 | SCA1 |
| AB015718 | 0.00482 | 0.006634 | 0.001172 | 0.202412 | STK10 |
| AF059575 | 0.00482 | 0.006634 | 0.000563 | −0.18074 | |
| M74089 | 0.00482 | 0.006634 | 0.00076 | 0.187888 | |
| U44111 | 0.00482 | 0.006634 | 0.003845 | 0.105361 | |
| AJ243310 | 0.00482 | 0.006921 | 0.000945 | −0.97643 | C14orf3 |
| W26854 | 0.00482 | 0.006921 | 0.011098 | −0.13774 | DKFZP434D156 |
| U88629 | 0.00482 | 0.006921 | 0.001778 | −0.16763 | ELL2 |
| M59830 | 0.00482 | 0.006921 | 0.000221 | −1.12882 | HSPA1B |
| M95929 | 0.00482 | 0.006921 | 0.004606 | −0.34536 | PMX1 |
| M57399 | 0.00482 | 0.006921 | 0.010231 | −0.14331 | PTN |
| N25117 | 0.00482 | 0.006921 | 0.002068 | −0.16335 | RPS26 |
| AL049940 | 0.00482 | 0.006921 | 0.001149 | −0.42489 | RYBP |
| U39318 | 0.00482 | 0.006921 | 0.001097 | −0.24533 | UBE2D3 |
| Z29331 | 0.00482 | 0.006921 | 0.000193 | −0.15851 | UBE2H |
| M55682 | 0.00482 | 0.006921 | 0.010264 | −0.10921 | |
| S58544 | 0.00482 | 0.006921 | 0.005501 | −0.11193 | |
| L13687 | 0.00482 | 0.007311 | 0.002185 | 0.114008 | ARL2 |
| M88714 | 0.00482 | 0.007311 | 0.002075 | 0.114833 | BDKRB2 |
| AL050173 | 0.00482 | 0.007311 | 0.001866 | 0.128954 | C21orf25 |
| M33680 | 0.00482 | 0.007311 | 0.002612 | 0.134487 | CD81 |
| X05299 | 0.00482 | 0.007311 | 0.003837 | 0.171613 | CENPB |
| X16832 | 0.00482 | 0.007311 | 0.000578 | 0.177395 | CTSH |
| U83410 | 0.00482 | 0.007311 | 0.005207 | 0.219569 | CUL2 |
| AL050018 | 0.00482 | 0.007311 | 0.003938 | 0.220539 | DKFZP564B116 |
| AL080063 | 0.00482 | 0.007311 | 0.006562 | 0.186332 | DKFZP564I052 |
| AL050286 | 0.00482 | 0.007311 | 0.000767 | 0.221397 | DKFZP586A011 |
| X63692 | 0.00482 | 0.007311 | 0.003174 | 0.172997 | DNMT1 |
| AA522537 | 0.00482 | 0.007311 | 0.002762 | 0.113812 | ELAC2 |
| AI183417 | 0.00482 | 0.007311 | 0.006167 | 0.101739 | GABPB1 |
| X62534 | 0.00482 | 0.007311 | 0.000973 | 0.195089 | HMG2 |
| D50532 | 0.00482 | 0.007311 | 0.001268 | 0.159735 | HML2 |
| AJ006591 | 0.00482 | 0.007311 | 0.001379 | 0.1682 | HSA6591 |
| Y00796 | 0.00482 | 0.007311 | 0.000438 | 0.386166 | ITGAL |
| AB018301 | 0.00482 | 0.007311 | 0.008701 | 0.138344 | KIAA0758 |
| AB020694 | 0.00482 | 0.007311 | 0.002526 | 0.205561 | KIAA0887 |
| AB023198 | 0.00482 | 0.007311 | 0.000409 | 0.275051 | KIAA0981 |
| AB028958 | 0.00482 | 0.007311 | 0.001533 | 0.117614 | KIAA1035 |
| U66711 | 0.00482 | 0.007311 | 0.006567 | 0.260368 | LY6E |
| L13744 | 0.00482 | 0.007311 | 0.007658 | 0.19599 | MLLT3 |
| Y09631 | 0.00482 | 0.007311 | 0.000769 | 0.309898 | PIBF1 |
| L77213 | 0.00482 | 0.007311 | 0.001122 | 0.247214 | PMVK |
| X73478 | 0.00482 | 0.007311 | 0.000681 | 0.242238 | PPP2R4 |
| U94319 | 0.00482 | 0.007311 | 0.000688 | 0.337656 | PSIP2 |
| U27516 | 0.00482 | 0.007311 | 0.000222 | 0.194938 | RAD52 |
| W25793 | 0.00482 | 0.007311 | 0.000438 | 0.258505 | RNF3 |
| X06617 | 0.00482 | 0.007311 | 0.002581 | 0.116631 | RPS11 |
| Z25749 | 0.00482 | 0.007311 | 0.001419 | 0.123333 | RPS7 |
| U80760 | 0.00482 | 0.007311 | 0.007371 | 0.161214 | TNRC1 |
| L27071 | 0.00482 | 0.007311 | 0.000638 | 0.372837 | TXK |
| AL031427 | 0.00482 | 0.007311 | 0.000541 | 0.367004 | |
| L109722 | 0.00482 | 0.007311 | 0.00187 | 0.134304 | |
| X15675 | 0.00482 | 0.007311 | 0.011165 | 0.131908 | |
| AL050089 | 0.00482 | 0.007852 | 0.001906 | −0.23061 | BAZ1A |
| L22005 | 0.00482 | 0.007852 | 0.002439 | −0.22532 | CDC34 |
| AB014679 | 0.00482 | 0.007852 | 0.003059 | −0.13664 | CHST2 |
| X77956 | 0.00482 | 0.007852 | 0.000689 | −0.22743 | ID1 |
| AI814466 | 0.00482 | 0.007852 | 0.001127 | −0.1955 | VAMP5 |
| HG4074-HT4344 | 0.00482 | 0.007852 | 0.000964 | −0.17461 | |
| AF005050 | 0.00482 | 0.008059 | 0.001761 | 0.230395 | DNPEP |
| J03909 | 0.00482 | 0.008059 | 0.000125 | −0.18353 | IFI30 |
| X59841 | 0.00482 | 0.008059 | 0.000226 | 0.265756 | PBX3 |
| AI819942 | 0.00482 | 0.009314 | 0.002286 | 0.326115 | 2-Sep |
| D86981 | 0.00482 | 0.009314 | 0.003441 | 0.319525 | APPBP2 |
| Y10805 | 0.00482 | 0.009314 | 0.002583 | 0.183496 | HRMT1L2 |
| U51127 | 0.00482 | 0.009314 | 0.00212 | 0.282678 | IRF5 |
| U14970 | 0.00482 | 0.009314 | 0.000561 | 0.144991 | RPS5 |
| AI813532 | 0.00482 | 0.009314 | 0.00037 | −0.41933 | TNFRSF1B |
| Y15228 | 0.00482 | 0.010363 | 0.002026 | −0.17032 | DLEU2 |
| AA926957 | 0.00482 | 0.010363 | 0.000909 | −0.22483 | FLJ10534 |
| AA554945 | 0.00482 | 0.010363 | 0.001827 | −0.14301 | FLJ10803 |
| AJ001383 | 0.00482 | 0.010363 | 0.001968 | −0.3226 | LY94 |
| M97676 | 0.00482 | 0.010363 | 0.010011 | −0.16313 | MSX1 |
| AF002020 | 0.00482 | 0.010363 | 0.001278 | −0.1736 | NPC1 |
| U25975 | 0.00482 | 0.010363 | 0.000764 | −0.24651 | PAK2 |
| X66363 | 0.00482 | 0.010363 | 0.000837 | −0.4179 | PCTK1 |
| D87957 | 0.00482 | 0.010363 | 0.004418 | −0.14751 | RQCD1 |
| AI610467 | 0.00482 | 0.010363 | 0.000699 | −0.17683 | SMG1 |
| AJ012008 | 0.00482 | 0.010363 | 0.002571 | −0.32997 | |
| AJ012008 | 0.00482 | 0.010363 | 0.001638 | −0.16204 | |

TABLE II
Gene Expression Profile from PBMCs of MS vs. Healthy-Highest
Scoring Genes (Bonfferoni analysis)

| | TNOM | Info | t-Test | Log | |
|---|---|---|---|---|---|
| Identifier | PValue | PValue | PValue | FoldChange | Symbol |
| AA203527 | 1.37131E−05 | 1.613E−06 | 1.18E−07 | 0.281992 | RPP20 |
| AA780049 | 7.44428E−05 | 2.35E−05 | 7.39E−07 | 0.54912 | FLJ21439 |
| AA845349 | 7.44428E−05 | 0.0001187 | 7.78E−07 | 0.457176 | TRIP7 |
| AA902713 | 2.10971E−06 | 1.993E−06 | 1.44E−06 | 0.474378 | |
| AB002344 | 7.44428E−05 | 5.915E−05 | 8.48E−07 | −1.00068 | KIAA0346 |
| AB002347 | 2.10971E−06 | 1.329E−07 | 7.19E−10 | 0.371731 | KIAA0349 |
| AB002348 | 1.37131E−05 | 3.861E−06 | 2.49E−07 | 0.576346 | KIAA0350 |
| AB002386 | 2.10971E−06 | 7.732E−07 | 5.34E−09 | 0.586117 | EZH1 |
| AB002448 | 1.37131E−05 | 5.009E−06 | 2.45E−07 | 0.468926 | |
| AB007891 | 1.37131E−05 | 3.861E−06 | 3.99E−05 | 0.196376 | KIAA0431 |
| AB007895 | 1.37131E−05 | 5.009E−06 | 9.61E−07 | 0.186643 | KIAA0435 |
| AB007927 | 2.10971E−06 | 1.993E−06 | 2.12E−07 | 0.323787 | RERE |
| AB007960 | 2.10971E−06 | 9.536E−07 | 7.96E−06 | 0.447772 | SH3GLB1 |
| AB008775 | 0.000344298 | 0.0003041 | 1.88E−06 | −0.80745 | AQP9 |
| AB011004 | 0.000344298 | 7.048E−06 | 1.41E−06 | −1.34073 | UAP1 |
| AB011108 | 1.37131E−05 | 5.045E−07 | 4.39E−07 | 0.453498 | PRP4 |
| AB011113 | 1.37131E−05 | 7.048E−06 | 3.74E−07 | 0.444795 | WDR7 |
| AB011115 | 1.37131E−05 | 5.045E−07 | 3.39E−07 | 0.382809 | KIAA0543 |
| AB011161 | 2.57282E−08 | 4.013E−09 | 9.64E−11 | 0.63432 | PIP5K1C |
| AB014535 | 1.37131E−05 | 5.045E−07 | 1.04E−06 | 0.285282 | KIAA0635 |
| AB014538 | 0.000344298 | 4.67E−05 | 1.98E−06 | −0.63923 | KIAA0638 |
| AB014579 | 1.37131E−05 | 5.009E−06 | 6.08E−08 | 0.367966 | MGEA5 |
| AB014608 | 7.44428E−05 | 9.644E−05 | 4.59E−06 | 0.41494 | KIAA0708 |
| AB015019 | 7.44428E−05 | 7.048E−06 | 2.75E−07 | −0.24515 | BAIAP2 |
| AB018343 | 1.83773E−09 | 4.157E−10 | 9.05E−12 | 0.383078 | KIAA0800 |
| AB023153 | 2.10971E−06 | 1.329E−07 | 1.82E−08 | 0.895842 | KIAA0936 |
| AB023235 | 7.44428E−05 | 1.613E−06 | 1.43E−05 | 0.311216 | KIAA1018 |
| AB026118 | 0.00137719 | 0.0001897 | 4.47E−06 | −0.24886 | MALT1 |
| AB026436 | 7.44428E−05 | 1.613E−06 | 0.000219 | −0.7589 | DUSP10 |
| AB028951 | 2.63714E−07 | 1.715E−07 | 8.78E−09 | 0.543028 | KIAA1028 |
| AB028981 | 2.10971E−06 | 7.732E−07 | 5.34E−07 | 0.282288 | KIAA1058 |
| AB029015 | 2.63714E−07 | 2.488E−07 | 5.37E−09 | 0.695063 | PLCE2 |
| AB029038 | 7.44428E−05 | 1.613E−06 | 7.62E−05 | 0.364386 | KIAA1115 |
| AC002400 | 1.37131E−05 | 3.06E−05 | 2.28E−06 | −0.25834 | UBPH |
| AF000545 | 7.44428E−05 | 5.226E−05 | 3.48E−06 | −0.85393 | P2Y10 |
| AF001294 | 1.37131E−05 | 7.048E−06 | 1.23E−06 | −0.76359 | TSSC3 |
| AF004230 | 2.63714E−07 | 1.715E−07 | 3.06E−07 | 0.349166 | LILRB1 |
| AF005043 | 7.44428E−05 | 5.226E−05 | 2.7E−06 | 0.408592 | PARG |
| AF007130 | 2.10971E−06 | 5.045E−07 | 2.51E−06 | 0.391811 | LOC54104 |
| AF007142 | 0.000344298 | 0.0004443 | 3.15E−06 | 0.678734 | |
| AF007151 | 1.37131E−05 | 5.045E−07 | 3.25E−06 | 0.468343 | MMS19L |
| AF010193 | 7.44428E−05 | 2.35E−05 | 1.26E−07 | −1.4705 | MADH7 |
| AF010309 | 1.37131E−05 | 5.009E−06 | 7.36E−07 | −0.28533 | PIG3 |
| AF012023 | 7.44428E−05 | 5.915E−05 | 1.02E−06 | 0.50623 | ICAP-1A |
| AF014958 | 2.10971E−06 | 4.309E−06 | 1.05E−07 | −0.42152 | CCRL2 |
| AF015553 | 2.10971E−06 | 9.536E−07 | 2.61E−07 | 0.61214 | GTF2I |
| AF019083 | 1.37131E−05 | 5.009E−06 | 8.34E−07 | 0.17011 | PTENP1 |
| AF022375 | 2.63714E−07 | 8.227E−08 | 1.87E−11 | −1.35847 | VEGF |
| AF023614 | 1.37131E−05 | 1.511E−05 | 4.79E−07 | −0.20744 | TACI |
| AF024710 | 8.54758E−11 | 8.548E−11 | 1.13E−12 | −1.95537 | VEGF |
| AF026086 | 0.000344298 | 4.67E−05 | 2.66E−06 | 0.297942 | PEX1 |
| AF029777 | 1.37131E−05 | 7.048E−06 | 8.27E−07 | 0.290159 | GCN5L2 |
| AF030249 | 1.37131E−05 | 1.613E−06 | 1.98E−07 | 0.534547 | ECH1 |
| AF035281 | 2.10971E−06 | 2.484E−06 | 4.87E−07 | 0.472445 | |
| AF038564 | 1.37131E−05 | 1.613E−06 | 2.05E−07 | −0.40446 | ITCH |
| AF040707 | 2.10971E−06 | 1.993E−06 | 3.57E−07 | 0.289845 | NPR2L |
| AF042386 | 1.37131E−05 | 5.009E−06 | 0.000107 | 0.137192 | PPIE |
| AF052160 | 7.44428E−05 | 1.511E−05 | 1.67E−06 | 0.623021 | |
| AF054176 | 2.10971E−06 | 1.329E−07 | 6.47E−09 | −0.58138 | C1orf7 |
| AF054589 | 0.000344298 | 2.35E−05 | 1.98E−06 | 0.945394 | |
| AF061258 | 7.44428E−05 | 9.644E−05 | 1.58E−06 | 0.622201 | LIM |
| AF067853 | 1.37131E−05 | 5.009E−06 | 5.02E−06 | 0.361707 | ADSL |
| AF069517 | 2.10971E−06 | 1.329E−07 | 4.91E−07 | 0.399638 | RBM6 |
| AF070582 | 2.63714E−07 | 1.715E−07 | 3.23E−08 | −0.19773 | MGC13033 |
| AF070606 | 1.37131E−05 | 3.06E−05 | 1.48E−06 | −0.89337 | |
| AF070617 | 1.37131E−05 | 3.861E−06 | 3.23E−07 | 0.323494 | |
| AF077820 | 2.63714E−07 | 2.188E−08 | 2.91E−08 | 0.656852 | LRP5 |
| AF079167 | 2.63714E−07 | 2.488E−07 | 7.37E−10 | −1.93249 | OLR1 |
| AF082557 | 0.00137719 | 4.67E−05 | 2.23E−06 | 0.226994 | TNKS |
| AF094481 | 1.37131E−05 | 5.009E−06 | 2.74E−07 | −0.29045 | CGGBP1 |
| AF098641 | 2.63714E−07 | 1.715E−07 | 1.56E−07 | −0.41172 | |
| AF110377 | 1.37131E−05 | 5.009E−06 | 3.05E−05 | 0.361232 | TRRAP |
| AF117829 | 7.44428E−05 | 0.000129 | 2.61E−06 | −0.57516 | RIPK2 |
| AI133727 | 7.44428E−05 | 0.0001187 | 1.43E−06 | 0.181464 | FLB6421 |
| AI141670 | 0.000344298 | 0.0003041 | 1.6E−06 | −0.2494 | CLCN2 |
| AI148772 | 0.000344298 | 4.67E−05 | 4.18E−06 | −1.02619 | KYNU |
| AI184802 | 2.63714E−07 | 2.188E−08 | 2.67E−09 | −0.21576 | HPRP4P |
| AI560890 | 2.57282E−08 | 2.829E−08 | 1.8E−07 | 0.179028 | |
| AI670100 | 7.44428E−05 | 2.577E−05 | 7.7E−07 | 0.22677 | GRLF1 |
| AI754391 | 1.37131E−05 | 3.861E−06 | 1.72E−06 | −0.27657 | KLF12 |
| AI935146 | 0.000344298 | 2.35E−05 | 2.05E−06 | −0.46726 | GALNT3 |
| AI950382 | 1.37131E−05 | 1.613E−06 | 1.63E−07 | −0.74128 | KIAA0585 |
| AI970189 | 0.000344298 | 2.35E−05 | 6.16E−07 | −0.75934 | KIAA0997 |
| AJ002190 | 7.44428E−05 | 1.613E−06 | 2.17E−08 | 0.33775 | GNPAT |
| AJ007042 | 2.63714E−07 | 1.715E−07 | 2.1E−07 | 0.170935 | WHSC1 |
| AJ010059 | 2.10971E−06 | 5.045E−07 | 2.95E−06 | 0.2235 | SIT |
| AL008583 | 2.63714E−07 | 2.188E−08 | 1.12E−08 | 0.250082 | CBX6 |
| AL021154 | 0.000344298 | 4.67E−05 | 2.19E−06 | −0.82935 | ID3 |
| AL021707 | 0.000344298 | 7.048E−06 | 4.95E−06 | −2.21462 | |
| AL022398 | 7.44428E−05 | 1.613E−06 | 8.09E−08 | 0.919627 | |
| AL022398 | 7.44428E−05 | 7.048E−06 | 1.1E−07 | 0.79713 | DJ434O14.3 |
| AL022398 | 7.44428E−05 | 2.577E−05 | 2.4E−06 | 0.493166 | |
| AL023553 | 1.37131E−05 | 1.753E−05 | 2.51E−06 | 0.226635 | PMM1 |
| AL049387 | 0.00137719 | 0.0001897 | 5.12E−06 | 0.379296 | |
| AL049409 | 7.44428E−05 | 1.511E−05 | 1.1E−06 | 0.714173 | LEF1 |
| AL049782 | 7.44428E−05 | 2.577E−05 | 7.66E−07 | 0.237794 | |
| AL049787 | 1.37131E−05 | 5.009E−06 | 7.11E−06 | 0.311278 | |
| AL049963 | 0.000344298 | 4.67E−05 | 8.36E−07 | −0.74421 | LOC64116 |
| AL050084 | 7.44428E−05 | 1.613E−06 | 5.26E−05 | 0.509331 | DC8 |
| AL050087 | 2.10971E−06 | 2.484E−06 | 1.27E−07 | −0.31279 | KIAA1785 |
| AL050196 | 1.37131E−05 | 5.009E−06 | 2E−05 | −0.24688 | DKFZP586D2223 |
| AL050281 | 0.000344298 | 0.0002051 | 2.85E−06 | 0.30517 | NAG |
| AL050353 | 0.000344298 | 2.35E−05 | 4.42E−06 | 0.179352 | OIP2 |
| AL050371 | 0.000344298 | 2.35E−05 | 3.7E−06 | 0.493288 | PISD |
| AL080071 | 0.000344298 | 0.0003041 | 3.12E−06 | 0.237367 | DKFZP564M082 |
| AL080141 | 1.37131E−05 | 5.009E−06 | 2.42E−07 | 0.330868 | SEC31B-1 |
| AL096780 | 1.37131E−05 | 5.045E−07 | 2.13E−06 | 0.34487 | CHKL |
| AW051579 | 1.37131E−05 | 1.613E−06 | 7.58E−07 | 0.593476 | FLJ10512 |
| D10704 | 1.37131E−05 | 1.753E−05 | 4.69E−07 | −0.36791 | CHK |
| D13891 | 2.10971E−06 | 2.484E−06 | 4.57E−05 | −0.20577 | ID2 |
| D30758 | 2.10971E−06 | 1.993E−06 | 1.58E−05 | 0.27738 | CENTB1 |
| D30783 | 2.57282E−08 | 2.829E−08 | 8.95E−10 | −1.65011 | EREG |
| D49677 | 7.44428E−05 | 7.048E−06 | 4.18E−06 | 0.198707 | U2AF1RS2 |
| D50406 | 1.37131E−05 | 3.861E−06 | 2.65E−05 | 0.461907 | RECK |
| D50525 | 0.000344298 | 4.67E−05 | 3.02E−06 | 0.486698 | |
| D78579 | 1.37131E−05 | 7.048E−06 | 4.25E−07 | −1.65638 | NR4A3 |
| D78579 | 7.44428E−05 | 7.048E−06 | 9.62E−07 | −1.61438 | NR4A3 |
| D80011 | 7.44428E−05 | 1.613E−06 | 4.2E−07 | −0.35073 | KIAA0189 |
| D87119 | 7.44428E−05 | 2.35E−05 | 1.8E−06 | 0.425625 | GS3955 |
| D87119 | 7.44428E−05 | 5.226E−05 | 4.62E−06 | 0.557116 | GS3955 |
| D87466 | 1.37131E−05 | 8.661E−06 | 1.49E−07 | 0.466046 | KIAA0276 |
| HG1103-HT1103 | 1.37131E−05 | 1.613E−06 | 1.16E−07 | −0.39165 | |
| HG2007-HT2056 | 7.44428E−05 | 9.644E−05 | 4.01E−06 | −0.41408 | |
| HG2724-HT2820 | 1.37131E−05 | 3.06E−05 | 5.17E−06 | −1.33814 | |
| HG3227-HT3404 | 2.63714E−07 | 1.715E−07 | 1.68E−08 | −0.25361 | |
| HG4582-HT4987 | 7.44428E−05 | 2.35E−05 | 4.63E−07 | −0.39588 | |
| J02939 | 7.44428E−05 | 1.613E−06 | 2.16E−07 | −0.87844 | SLC3A2 |
| J02973 | 1.37131E−05 | 5.045E−07 | 2.93E−07 | −1.30804 | THBD |
| J03258 | 0.000344298 | 0.0001695 | 1.21E−06 | −0.58295 | VDR |
| J04130 | 0.000344298 | 2.35E−05 | 3.02E−06 | −0.62071 | SCYA4 |
| L04733 | 0.00137719 | 2.35E−05 | 8.84E−07 | 0.306455 | KNS2 |
| L05424 | 2.10971E−06 | 1.329E−07 | 2.27E−09 | −0.58081 | CD44 |
| L12002 | 7.44428E−05 | 4.67E−05 | 1.23E−06 | 0.286717 | ITGA4 |
| L13740 | 2.63714E−07 | 2.188E−08 | 5.83E−08 | −1.45891 | NR4A1 |
| L13740 | 1.37131E−05 | 5.009E−06 | 9.1E−08 | −0.61928 | NR4A1 |
| L13773 | 1.37131E−05 | 1.753E−05 | 6.44E−07 | 0.247919 | MLLT2 |
| L16499 | 1.37131E−05 | 8.661E−06 | 5.12E−06 | 0.374296 | HHEX |
| L20941 | 2.63714E−07 | 1.329E−07 | 1.78E−06 | −0.58618 | FTH1 |
| L22075 | 2.63714E−07 | 2.488E−07 | 1.1E−08 | −0.55736 | GNA13 |
| L22569 | 1.37131E−05 | 8.661E−06 | 1.52E−06 | 0.318129 | CTSB |
| L25665 | 0.000344298 | 0.0001695 | 3.34E−06 | −0.4513 | GNL1 |
| L33881 | 2.63714E−07 | 1.715E−07 | 5.06E−08 | −0.59585 | PRKCI |
| L40377 | 1.37131E−05 | 5.045E−07 | 3.49E−07 | −0.79409 | SERPINB8 |
| L47738 | 2.57282E−08 | 4.013E−09 | 7.54E−09 | 0.31646 | PIR121 |
| L78132 | 7.44428E−05 | 5.226E−05 | 5.15E−07 | 0.358576 | LGALS8 |
| M12267 | 0.000344298 | 0.0001695 | 4.07E−06 | −0.3279 | OAT |
| M12959 | 7.44428E−05 | 2.577E−05 | 1.61E−06 | 0.128482 | TRA@ |
| M15330 | 8.54758E−11 | 8.548E−11 | 2.49E−12 | −2.13825 | IL1B |
| M17017 | 7.44428E−05 | 0.0001187 | 1.43E−06 | −1.74073 | IL8 |
| M22919 | 2.63714E−07 | 3.119E−07 | 9.52E−08 | −0.81053 | MYL6 |
| M23114 | 2.10971E−06 | 4.309E−06 | 1.59E−07 | −0.96141 | ATP2A2 |
| M24194 | 7.44428E−05 | 1.613E−06 | 4.38E−06 | 0.560895 | GNB2L1 |
| M24283 | 0.000344298 | 4.67E−05 | 3.71E−06 | −1.32611 | ICAM1 |
| M24895 | 2.10971E−06 | 1.329E−07 | 1.72E−08 | 0.476779 | AMY2B |
| M26683 | 7.44428E−05 | 0.0001187 | 3.7E−06 | −0.16179 | SCYA2 |
| M27492 | 0.000344298 | 0.0004017 | 2.01E−06 | −0.32619 | IL1R1 |
| M28130 | 7.44428E−05 | 4.67E−05 | 8.02E−07 | −2.27292 | IL8 |
| M31165 | 7.44428E−05 | 5.226E−05 | 1.38E−06 | −0.34617 | TNFAIP6 |
| M31523 | 1.37131E−05 | 1.753E−05 | 2.09E−06 | 0.36898 | TCF3 |
| M36821 | 1.37131E−05 | 8.661E−06 | 2.21E−07 | −0.36334 | GRO3 |
| M55153 | 7.44428E−05 | 2.577E−05 | 4.77E−06 | −0.27465 | TGM2 |
| M58603 | 7.44428E−05 | 5.226E−05 | 1.28E−06 | −0.73537 | NFKB1 |
| M59040 | 0.00137719 | 2.35E−05 | 2.82E−06 | −0.46271 | CD44 |
| M60784 | 7.44428E−05 | 5.226E−05 | 1.24E−06 | 0.559903 | SNRPA |
| M60922 | 7.44428E−05 | 1.511E−05 | 4.47E−08 | 0.39657 | FLOT2 |
| M62403 | 7.44428E−05 | 5.226E−05 | 5.57E−07 | −0.53749 | IGFBP4 |
| M63256 | 0.000344298 | 5.915E−05 | 6.54E−07 | 0.454561 | CDR2 |
| M63904 | 2.57282E−08 | 1.031E−08 | 5.38E−09 | −0.59612 | GNA15 |
| M63978 | 0.000344298 | 4.67E−05 | 1.77E−06 | −0.44762 | VEGF |
| M64571 | 1.83773E−09 | 1.838E−09 | 2.41E−11 | 0.416659 | MAP4 |
| M69199 | 2.10971E−06 | 1.993E−06 | 1.45E−07 | −1.9021 | G0S2 |
| M73547 | 1.37131E−05 | 5.009E−06 | 9.2E−08 | 0.438897 | D5S346 |
| M74525 | 2.10971E−06 | 2.484E−06 | 3.5E−07 | −0.61792 | UBE2B |
| M80244 | 0.000344298 | 7.048E−06 | 2.72E−06 | −0.8522 | SLC7A5 |
| M84443 | 1.37131E−05 | 5.045E−07 | 4.08E−07 | 0.303567 | GALK2 |
| M94856 | 7.44428E−05 | 5.226E−05 | 4.99E−06 | −0.23847 | FABP5 |
| M95678 | 0.000344298 | 7.048E−06 | 2E−06 | 0.432923 | PLCB2 |
| M98833 | 7.44428E−05 | 1.613E−06 | 1.52E−06 | 0.434288 | FLI1 |
| N23137 | 2.10971E−06 | 2.484E−06 | 2.06E−07 | 0.247311 | MPHOSPH9 |
| N23137 | 0.00137719 | 0.0001695 | 4.12E−06 | 0.244083 | MPHOSPH9 |
| N30151 | 7.44428E−05 | 1.613E−06 | 5.05E−05 | 0.393521 | STX16 |
| N42007 | 2.10971E−06 | 2.484E−06 | 9.19E−05 | 0.167986 | NUP50 |
| N53547 | 7.44428E−05 | 8.556E−05 | 1.8E−07 | 0.296678 | MGC5508 |
| N90862 | 1.37131E−05 | 5.045E−07 | 3.28E−08 | 0.43576 | VAMP8 |
| N90866 | 2.63714E−07 | 8.227E−08 | 2.76E−08 | 0.304525 | CDW52 |
| N98667 | 1.37131E−05 | 8.661E−06 | 3.38E−07 | 0.367127 | KIAA1696 |
| R90942 | 1.37131E−05 | 5.009E−06 | 1.05E−05 | −0.17696 | ST6GALNACIV |
| S52028 | 2.10971E−06 | 5.045E−07 | 9.62E−08 | −0.81662 | CTH |
| S68134 | 0.000344298 | 7.048E−06 | 8.37E−07 | −1.64652 | CREM |
| S68134 | 0.000344298 | 7.048E−06 | 4.35E−06 | −2.47105 | CREM |
| S68271 | 0.000344298 | 7.048E−06 | 3.03E−06 | −2.07185 | CREM |
| S73591 | 1.37131E−05 | 1.511E−05 | 4.68E−06 | 0.414777 | VDUP1 |
| S76638 | 7.44428E−05 | 2.35E−05 | 7.47E−07 | −0.35416 | NFKB2 |
| S78187 | 7.44428E−05 | 1.613E−06 | 1.95E−05 | 0.203265 | CDC25B |
| S78771 | 0.000344298 | 5.915E−05 | 2.55E−06 | −0.31389 | BRD2 |
| S81914 | 0.000344298 | 7.048E−06 | 4.18E−07 | −1.59146 | IER3 |
| U02020 | 1.37131E−05 | 8.661E−06 | 1.37E−06 | −1.13863 | PBEF |
| U02570 | 1.37131E−05 | 2.813E−05 | 1.26E−06 | 0.432431 | ARHGAP1 |
| U03634 | 1.37131E−05 | 1.753E−05 | 1E−06 | −0.21467 | LBC |
| U04636 | 0.000344298 | 5.915E−05 | 2.81E−06 | −1.85123 | PTGS2 |
| U05681 | 7.44428E−05 | 5.226E−05 | 3.37E−06 | −0.35383 | BCL3 |
| U07563 | 7.44428E−05 | 2.35E−05 | 4.91E−07 | −0.25016 | ABL1 |
| U09937 | 1.83773E−09 | 4.157E−10 | 2.04E−09 | −1.21578 | PLAUR |
| U10117 | 7.44428E−05 | 1.511E−05 | 4.07E−06 | 0.563673 | SCYE1 |
| U11732 | 1.37131E−05 | 3.861E−06 | 3.04E−07 | −0.22574 | ETV6 |
| U12767 | 7.44428E−05 | 1.613E−06 | 2.84E−07 | −1.23483 | NR4A3 |
| U12767 | 0.000344298 | 7.048E−06 | 2.55E−07 | −2.13744 | NR4A3 |
| U13695 | 7.44428E−05 | 1.613E−06 | 1.11E−05 | 0.805607 | PMS1 |
| U15552 | 1.37131E−05 | 5.009E−06 | 1.67E−05 | −0.68094 | HSU15552 |
| U17760 | 0.000344298 | 7.048E−06 | 4.25E−06 | −0.84472 | LAMB3 |
| U18300 | 7.44428E−05 | 0.000129 | 2.43E−06 | 0.183171 | DDB2 |
| U20982 | 2.10971E−06 | 1.993E−06 | 1.2E−08 | −0.67125 | IGFBP4 |
| U24166 | 7.44428E−05 | 1.613E−06 | 7.52E−06 | −0.45293 | MAPRE1 |
| U28811 | 0.000344298 | 7.048E−06 | 1.33E−06 | 0.32855 | GLG1 |
| U29171 | 1.37131E−05 | 5.009E−06 | 1.1E−06 | −0.6032 | CSNK1D |
| U29175 | 1.37131E−05 | 8.661E−06 | 1.9E−06 | 0.266342 | SMARCA4 |
| U29185 | 2.10971E−06 | 7.732E−07 | 1.56E−07 | −1.08006 | PRNP |
| U29344 | 2.10971E−06 | 9.536E−07 | 2.35E−07 | −0.43842 | FASN |
| U29656 | 2.10971E−06 | 7.732E−07 | 7.52E−08 | 0.353186 | NME3 |
| U29656 | 7.44428E−05 | 0.000129 | 4.31E−06 | 0.471876 | NME3 |
| U32324 | 1.37131E−05 | 5.045E−07 | 3.21E−08 | 0.334966 | IL11RA |
| U33017 | 2.63714E−07 | 1.715E−07 | 5.2E−07 | 0.373581 | SLAM |
| U38847 | 7.44428E−05 | 2.35E−05 | 9.91E−07 | 0.222946 | TARBP1 |
| U41815 | 1.37131E−05 | 5.045E−07 | 2.16E−07 | −0.96931 | NUP98 |
| U43774 | 0.000344298 | 2.35E−05 | 8.8E−07 | −0.39938 | FCAR |
| U44839 | 2.10971E−06 | 9.536E−07 | 2.54E−07 | −0.97008 | USP11 |
| U47414 | 2.10971E−06 | 9.536E−07 | 2.31E−06 | 0.370736 | CCNG2 |
| U47927 | 2.57282E−08 | 2.829E−08 | 5.53E−09 | 0.545592 | USP5 |
| U48807 | 1.37131E−05 | 5.009E−06 | 4.97E−08 | −0.93178 | DUSP4 |
| U49187 | 7.44428E−05 | 1.511E−05 | 1.48E−06 | 0.671467 | C6orf32 |
| U49187 | 7.44428E−05 | 9.644E−05 | 3.53E−06 | 0.511392 | C6orf32 |
| U49844 | 7.44428E−05 | 7.048E−06 | 3.67E−07 | 0.47168 | ATR |
| U50527 | 1.37131E−05 | 5.009E−06 | 5.11E−06 | 0.416543 | |
| U50928 | 7.44428E−05 | 1.613E−06 | 4.72E−06 | 0.302213 | PKD2 |
| U51007 | 7.44428E−05 | 1.511E−05 | 1.49E−06 | 0.309996 | PSMD4 |
| U51205 | 1.37131E−05 | 5.045E−07 | 2.65E−07 | −0.76279 | COP9 |
| U51478 | 7.44428E−05 | 2.35E−05 | 6.1E−07 | −0.58 | ATP1B3 |
| U51920 | 2.10971E−06 | 1.329E−07 | 7.01E−08 | −0.28142 | SRP54 |
| U52960 | 2.10971E−06 | 1.613E−06 | 1.51E−07 | −0.84863 | SURB7 |
| U56998 | 0.000344298 | 7.048E−06 | 3.7E−06 | −0.74294 | CNK |
| U64197 | 1.83773E−09 | 1.838E−09 | 2.95E−10 | −0.62373 | SCYA20 |
| U65928 | 7.44428E−05 | 4.67E−05 | 2.85E−07 | 0.408918 | COPS5 |
| U66063 | 2.10971E−06 | 2.484E−06 | 4.7E−07 | 0.277185 | CAMK2G |
| U70735 | 1.37131E−05 | 8.661E−06 | 1.82E−06 | 0.249185 | MOV34-34KD |
| U72066 | 2.57282E−08 | 1.031E−08 | 4.33E−08 | −0.34482 | RBBP8 |
| U75968 | 2.10971E−06 | 1.993E−06 | 4.36E−06 | 0.139542 | DDX11 |
| U78107 | 8.54758E−11 | 3.691E−11 | 4.04E−12 | −0.43769 | NAPG |
| U78302 | 2.63714E−07 | 1.715E−07 | 2.41E−08 | 0.329878 | DECR1 |
| U78798 | 2.57282E−08 | 4.013E−09 | 1.11E−06 | −0.3172 | TRAF6 |
| U84007 | 7.44428E−05 | 1.613E−06 | 0.000235 | 0.236422 | AGL |
| U85245 | 7.44428E−05 | 1.613E−06 | 4.57E−07 | 0.365266 | PIP5K2B |
| U88629 | 0.000344298 | 4.67E−05 | 9.58E−07 | −0.32607 | ELL2 |
| U90917 | 1.37131E−05 | 1.613E−06 | 3.89E−07 | 0.433406 | FOXM1 |
| U91543 | 2.63714E−07 | 3.119E−07 | 2.01E−07 | 0.478678 | CHD3 |
| U91616 | 1.37131E−05 | 5.045E−07 | 1.27E−07 | −0.80419 | NFKBIE |
| U96876 | 7.44428E−05 | 1.613E−06 | 3.54E−06 | −0.45317 | INSIG1 |
| U97105 | 1.37131E−05 | 1.753E−05 | 6.56E−07 | 1.00615 | DPYSL2 |
| W28319 | 1.37131E−05 | 5.009E−06 | 1.5E−05 | 0.294631 | FBLN1 |
| W28612 | 1.37131E−05 | 5.009E−06 | 1.7E−06 | −0.25519 | |
| W28743 | 0.000344298 | 7.048E−06 | 2.78E−06 | −0.28926 | PP1628 |
| X00737 | 2.10971E−06 | 9.536E−07 | 5.21E−08 | −0.67074 | NP |
| X02152 | 1.37131E−05 | 5.045E−07 | 4.63E−08 | −0.75601 | LDHA |
| X04366 | 1.37131E−05 | 2.813E−05 | 5.11E−06 | 0.346076 | CAPN1 |
| X04500 | 2.63714E−07 | 1.715E−07 | 3.43E−10 | −2.12121 | IL1B |
| X06256 | 1.37131E−05 | 2.35E−05 | 4.89E−07 | −0.7357 | ITGA5 |
| X13403 | 7.44428E−05 | 5.915E−05 | 4.21E−07 | 0.146032 | POU2F1 |
| X15217 | 7.44428E−05 | 4.67E−05 | 3.77E−07 | −0.2371 | SKIL |
| X15218 | 8.54758E−11 | 8.548E−11 | 1.4E−10 | −1.41501 | SKI |
| X16396 | 0.000344298 | 0.0002051 | 3.27E−06 | −0.6151 | MTHFD2 |
| X16706 | 7.44428E−05 | 1.613E−06 | 1.23E−06 | −1.09747 | FOSL2 |
| X53586 | 1.37131E−05 | 8.661E−06 | 3.4E−07 | 0.51291 | ITGA6 |
| X58141 | 7.44428E−05 | 9.644E−05 | 1.75E−06 | 0.384254 | ADD1 |
| X61123 | 7.44428E−05 | 0.0001057 | 4.17E−07 | −1.15256 | BTG1 |
| X61498 | 7.44428E−05 | 1.613E−06 | 8.8E−07 | −0.49884 | NFKB2 |
| X62535 | 1.37131E−05 | 1.613E−06 | 5.68E−07 | 0.243937 | DGKA |
| X63368 | 2.10971E−06 | 5.045E−07 | 2.3E−08 | −0.55432 | DNAJB2 |
| X64330 | 7.44428E−05 | 7.048E−06 | 2.27E−06 | 0.297851 | ACLY |
| X66363 | 2.63714E−07 | 1.715E−07 | 6.53E−07 | −0.24505 | PCTK1 |
| X66436 | 0.000344298 | 8.556E−05 | 1.88E−06 | −0.26662 | |
| X66945 | 7.44428E−05 | 1.511E−05 | 1.91E−07 | −0.35494 | FGFR1 |
| X68452 | 2.57282E−08 | 4.013E−09 | 9.12E−11 | −0.26618 | CCND2 |
| X69392 | 2.63714E−07 | 1.329E−07 | 1.1E−08 | 0.297444 | RPL26 |
| X70218 | 1.37131E−05 | 3.06E−05 | 2.44E−06 | −0.74691 | PPP4C |
| X74039 | 1.83773E−09 | 4.157E−10 | 1.51E−10 | −0.67381 | PLAUR |
| X79882 | 1.37131E−05 | 5.045E−07 | 1.78E−07 | 0.520965 | MVP |
| X82153 | 7.44428E−05 | 1.613E−06 | 2.27E−06 | 0.47844 | CTSK |
| X82209 | 2.10971E−06 | 5.045E−07 | 1.37E−09 | −0.45281 | MN1 |
| X87949 | 7.44428E−05 | 1.613E−06 | 4.05E−07 | −0.54468 | HSPA5 |
| X98172 | 7.44428E−05 | 4.67E−05 | 5.29E−07 | 0.507556 | CASP8 |
| X99142 | 1.37131E−05 | 8.661E−06 | 1.24E−06 | −0.29773 | KRTHB6 |
| X99656 | 1.37131E−05 | 5.045E−07 | 1.68E−06 | −0.23553 | SH3GL1 |
| Y00630 | 2.57282E−08 | 3.695E−08 | 6.65E−09 | −2.38485 | SERPINB2 |
| Y08683 | 1.37131E−05 | 5.045E−07 | 4.71E−06 | 0.492738 | CPT1B |
| Y14768 | 1.37131E−05 | 5.045E−07 | 7.26E−08 | 0.248383 | LTB |
| Y18004 | 1.37131E−05 | 5.009E−06 | 4.19E−07 | −0.9465 | SCML2 |
| Z11697 | 1.37131E−05 | 5.045E−07 | 3.55E−06 | −1.21033 | CD83 |
| Z14000 | 0.000344298 | 0.0002051 | 3.91E−06 | −0.33734 | RING1 |
| Z24724 | 2.63714E−07 | 2.188E−08 | 5.96E−09 | −1.10426 | |
| Z32860 | 1.37131E−05 | 5.009E−06 | 7.81E−06 | 0.133192 | |
| Z93930 | 2.63714E−07 | 2.488E−07 | 2.42E−05 | −0.39839 | XBP1 |

TABLE III
Differential Gene Expression in acute MS relapse vs. remission

| | TNOM | Info | t-Test | Log Fold | |
|---|---|---|---|---|---|
| Identifier | PValue | PValue | PValue | Change | Symbol |
| AI828210 | 5.38E−06 | 5.38E−06 | 8.37E−06 | −0.18947 | KIAA0284 |
| D14710 | 6.73E−05 | 3.19E−05 | 2.89E−05 | −0.35496 | ATP5A1 |
| U46692 | 6.73E−05 | 3.19E−05 | 0.000284 | −0.49741 | CSTB |
| AF061261 | 6.73E−05 | 3.19E−05 | 3.22E−05 | −0.28274 | MBLL |
| U51712 | 6.73E−05 | 3.19E−05 | 0.003464 | −0.42775 | SMAP31 |
| AB014558 | 6.73E−05 | 4.25E−05 | 0.000473 | 0.694784 | CRY2 |
| AB007936 | 6.73E−05 | 4.25E−05 | 0.000958 | −0.25409 | KIAA0467 |
| AC002115 | 6.73E−05 | 4.25E−05 | 0.000147 | 0.622841 | MGC10433 |
| AF052160 | 6.73E−05 | 4.25E−05 | 0.000182 | −0.46468 | |
| S78085 | 0.000538 | 0.000104 | 0.000102 | −0.55064 | PDCD2 |
| AL096719 | 0.000538 | 0.000104 | 0.000089 | −0.22287 | PFN2 |
| U61234 | 0.000538 | 0.000104 | 0.000844 | 0.299182 | TBCC |
| X12451 | 0.000538 | 0.000251 | 0.000876 | 1.04444 | CTSL |
| M35531 | 0.000538 | 0.000251 | 0.000241 | −0.20303 | FUT1 |
| M64174 | 0.000538 | 0.000251 | 3.43E−05 | −0.5508 | JAK1 |
| AB018269 | 0.000538 | 0.000251 | 7.39E−05 | −0.18186 | KIAA0726 |
| R92331 | 0.000538 | 0.000251 | 0.000104 | 0.289994 | MT1E |
| U19487 | 0.000538 | 0.000251 | 0.001738 | −0.25888 | PTGER2 |
| AF040965 | 0.000538 | 0.000251 | 0.000775 | 0.48898 | RES4-25 |
| U07563 | 0.000538 | 0.000251 | 3.61E−05 | −0.16779 | RRP4 |
| L40377 | 0.000538 | 0.000251 | 0.009479 | 0.452416 | SERPINB8 |
| AL080234 | 0.000538 | 0.000251 | 0.000377 | −0.52631 | |
| AJ242015 | 0.003096 | 0.00039 | 0.013957 | 0.281618 | ADAM28 |
| D86324 | 0.003096 | 0.00039 | 0.001801 | −0.34728 | CMAH |
| M94065 | 0.003096 | 0.00039 | 0.002391 | −0.13976 | DHODH |
| AC004382 | 0.003096 | 0.00039 | 0.000121 | −0.20383 | DKFZP434K046 |
| X54326 | 0.003096 | 0.00039 | 0.002734 | −0.39559 | EPRS |
| W25921 | 0.003096 | 0.00039 | 9.41E−05 | −0.39027 | GNS |
| X92110 | 0.003096 | 0.00039 | 0.000103 | −1.00581 | HCGVIII-1 |
| W28589 | 0.003096 | 0.00039 | 0.000225 | −0.20949 | HSPD1 |
| S66213 | 0.003096 | 0.00039 | 0.000134 | −0.28606 | ITGA6 |
| AB011158 | 0.000538 | 0.00039 | 0.000047 | −0.163 | KIAA0586 |
| AB023209 | 0.003096 | 0.00039 | 0.003354 | −0.09151 | KIAA0992 |
| AF035940 | 0.003096 | 0.00039 | 0.008457 | 0.282437 | MAGOH |
| M31724 | 0.003096 | 0.00039 | 0.000671 | 0.569343 | PTPN1 |
| X74262 | 0.003096 | 0.00039 | 0.000062 | −0.37623 | RBBP4 |
| J05249 | 0.003096 | 0.00039 | 0.00045 | −0.52346 | RPA2 |
| M55531 | 0.003096 | 0.00039 | 0.023054 | −0.22329 | SLC2A5 |
| AI865431 | 0.003096 | 0.00039 | 0.00027 | 0.423067 | TNFRSF5 |
| W28203 | 0.003096 | 0.00039 | 0.007983 | −0.17484 | |
| W28667 | 0.003096 | 0.00039 | 0.000846 | −0.49488 | |
| D13628 | 0.000538 | 0.000529 | 0.034335 | −0.10398 | ANGPT1 |
| U03271 | 0.000538 | 0.000529 | 0.000286 | −0.1675 | CAPZB |
| U05259 | 0.000538 | 0.000529 | 0.003589 | 0.551328 | CD79A |
| L13278 | 0.000538 | 0.000529 | 7.27E−05 | −0.43636 | CRYZ |
| M91670 | 0.000538 | 0.000529 | 0.003472 | 0.600255 | E2-EPF |
| AB029030 | 0.000538 | 0.000529 | 0.000657 | −0.13458 | KIAA1107 |
| AF016098 | 0.000538 | 0.000529 | 0.000433 | −0.16189 | NRP2 |
| X76091 | 0.000538 | 0.000529 | 0.004691 | 0.161349 | RFX2 |
| U52191 | 0.000538 | 0.000529 | 0.00229 | 1.2356 | SMCY |
| AA203345 | 0.000538 | 0.000529 | 0.001228 | −0.50409 | STX16 |
| U96113 | 0.000538 | 0.000529 | 0.000394 | −0.41425 | WWP1 |
| AL050263 | 0.000538 | 0.000529 | 0.000224 | −0.15981 | |
| Z48579 | 0.000538 | 0.000799 | 0.000184 | −0.30836 | ADAM10 |
| M31452 | 0.000538 | 0.000799 | 0.002899 | −0.13022 | C4BPA |
| AC003107 | 0.000538 | 0.000799 | 0.000262 | −0.16818 | COMP |
| M91670 | 0.000538 | 0.000799 | 0.000792 | 0.41925 | E2-EPF |
| AB023235 | 0.000538 | 0.000799 | 0.001348 | −0.30138 | KIAA1018 |
| X89960 | 0.000538 | 0.000799 | 0.026837 | −0.35169 | MCSP |
| D55654 | 0.000538 | 0.000799 | 0.019331 | −0.2254 | MDH1 |
| U02683 | 0.000538 | 0.000799 | 0.030035 | −0.09324 | NRF1 |
| S90469 | 0.000538 | 0.000799 | 0.000785 | 0.23032 | POR |
| AF020543 | 0.000538 | 0.000799 | 0.004286 | −0.25061 | PPT2 |
| M34181 | 0.000538 | 0.000799 | 0.000055 | −0.5883 | PRKACB |
| AF095448 | 0.000538 | 0.000799 | 0.000588 | −0.24961 | RAI3 |
| AF027150 | 0.000538 | 0.000799 | 0.000979 | −0.16012 | SIP1 |
| X02344 | 0.000538 | 0.000799 | 0.000918 | 0.430531 | TUBB2 |
| X02344 | 0.000538 | 0.000799 | 0.002225 | 0.296682 | TUBB2 |
| AI701164 | 0.000538 | 0.000799 | 0.000115 | −0.23639 | UBE2G1 |
| U96113 | 0.000538 | 0.000799 | 9.77E−05 | −0.45711 | WWP1 |
| AF016052 | 0.000538 | 0.000799 | 0.001254 | −0.19092 | ZNF24 |
| U21551 | 0.003096 | 0.00103 | 0.000836 | 0.278219 | BCAT1 |
| X77794 | 0.003096 | 0.00103 | 3.72E−05 | −0.81938 | CCNG1 |
| AF070530 | 0.003096 | 0.00103 | 0.014908 | 0.276942 | CL24751 |
| AB002331 | 0.003096 | 0.00103 | 0.001714 | −0.17304 | DATF1 |
| AI004207 | 0.003096 | 0.00103 | 0.000762 | −0.1648 | FLJ00002 |
| L76200 | 0.003096 | 0.00103 | 0.000824 | 0.444479 | GUK1 |
| U26398 | 0.003096 | 0.00103 | 0.001182 | −0.29185 | INPP4A |
| U69883 | 0.003096 | 0.00103 | 0.007922 | 0.103614 | KCNN1 |
| M13452 | 0.003096 | 0.00103 | 0.000467 | 0.405856 | LMNA |
| AA126505 | 0.003096 | 0.00103 | 0.002 | −0.39781 | NCAM1 |
| U88620 | 0.003096 | 0.00103 | 0.007562 | −0.3532 | OGG1 |
| M33336 | 0.003096 | 0.00103 | 0.001568 | −0.26454 | PRKAR1A |
| AB015982 | 0.003096 | 0.00103 | 0.000382 | −0.27486 | PRKCN |
| H68340 | 0.003096 | 0.00103 | 0.001222 | 0.516352 | RNAHP |
| M28225 | 0.003096 | 0.00103 | 0.000686 | 1.0733 | SCYA2 |
| X97064 | 0.003096 | 0.00103 | 0.003207 | −0.19906 | SEC23A |
| X68560 | 0.003096 | 0.00103 | 0.007856 | 0.437567 | SP3 |
| AF064094 | 0.003096 | 0.00103 | 0.000287 | −0.19385 | TADA2L |
| AB007872 | 0.003096 | 0.00103 | 0.000119 | −0.20778 | ZNF264 |
| W28255 | 0.013622 | 0.001698 | 0.001407 | −0.24426 | 76P |
| AB007934 | 0.003096 | 0.001698 | 0.003182 | −0.24405 | ACF7 |
| AL049954 | 0.013622 | 0.001698 | 0.024193 | −0.25818 | AHCYL1 |
| U90546 | 0.003096 | 0.001698 | 0.000105 | −0.34074 | BTN3A2 |
| AL035291 | 0.013622 | 0.001698 | 0.007668 | 0.506107 | CH1 |
| AF031647 | 0.013622 | 0.001698 | 0.004755 | 0.257244 | COPS3 |
| M57888 | 0.003096 | 0.001698 | 0.004549 | −0.64384 | CTLA1 |
| AF000987 | 0.003096 | 0.001698 | 0.009455 | 0.247586 | EIF1AY |
| U55766 | 0.003096 | 0.001698 | 0.00066 | 0.795017 | HRB2 |
| L12002 | 0.013622 | 0.001698 | 0.005765 | −0.1942 | ITGA4 |
| D14661 | 0.013622 | 0.001698 | 0.011324 | 0.391267 | KIAA0105 |
| D63875 | 0.013622 | 0.001698 | 0.002192 | −0.36411 | KIAA0155 |
| AB018285 | 0.013622 | 0.001698 | 0.001545 | 0.550994 | KIAA0742 |
| AB023180 | 0.013622 | 0.001698 | 0.001642 | 0.253479 | KIAA0963 |
| AL080102 | 0.013622 | 0.001698 | 0.003651 | 0.435751 | KIAA1856 |
| M22637 | 0.013622 | 0.001698 | 0.003792 | −0.27794 | LYL1 |
| D85131 | 0.013622 | 0.001698 | 0.005126 | −0.12291 | MAZ |
| D37965 | 0.013622 | 0.001698 | 0.01111 | −0.09143 | PDGFRL |
| Y18207 | 0.003096 | 0.001698 | 0.003474 | −0.17238 | PPP1R3C |
| L49229 | 0.013622 | 0.001698 | 0.000336 | −0.36639 | RB1 |
| U77664 | 0.013622 | 0.001698 | 0.002354 | 0.193666 | RPP38 |
| AL040137 | 0.003096 | 0.001698 | 0.008384 | −0.23366 | SAP18 |
| D31764 | 0.013622 | 0.001698 | 0.01295 | −0.13299 | SNX17 |
| X57655 | 0.013622 | 0.001698 | 0.002476 | −0.17382 | SPINK2 |
| M19267 | 0.013622 | 0.001698 | 0.013582 | 0.262886 | TPM1 |
| M12959 | 0.013622 | 0.001698 | 0.003907 | −0.08942 | TRA@ |
| AA160724 | 0.013622 | 0.001698 | 0.005695 | 0.267002 | |
| U37122 | 0.003096 | 0.002135 | 0.000571 | −0.59281 | ADD3 |
| AA903720 | 0.003096 | 0.002135 | 0.002557 | 0.244618 | BAP29 |
| M93107 | 0.003096 | 0.002135 | 0.00187 | −0.19146 | BDH |
| M17754 | 0.003096 | 0.002135 | 0.010333 | −0.10769 | BN51T |
| X15882 | 0.003096 | 0.002135 | 0.0023 | 0.227769 | COL6A2 |
| D15057 | 0.003096 | 0.002135 | 0.002814 | −0.26776 | DAD1 |
| S62138 | 0.003096 | 0.002135 | 0.002442 | 1.1158 | DDIT3 |
| AB026436 | 0.003096 | 0.002135 | 0.011189 | 0.711919 | DUSP10 |
| W27152 | 0.003096 | 0.002135 | 0.009498 | −0.1614 | FLJ10569 |
| AB001106 | 0.003096 | 0.002135 | 0.002408 | 0.444617 | GMFB |
| D87120 | 0.003096 | 0.002135 | 0.00475 | 0.236706 | GS3786 |
| AI200373 | 0.003096 | 0.002135 | 0.003822 | −0.31066 | H2AFI |
| U15085 | 0.003096 | 0.002135 | 0.011743 | 0.328857 | HLA-DMB |
| U90549 | 0.003096 | 0.002135 | 0.001654 | −0.26437 | HMG17L3 |
| AI760162 | 0.003096 | 0.002135 | 0.001313 | −0.47775 | HT012 |
| AB018306 | 0.003096 | 0.002135 | 0.000371 | 0.316202 | KIAA0763 |
| D14696 | 0.003096 | 0.002135 | 0.016949 | 0.259239 | LAPTM4A |
| U23852 | 0.003096 | 0.002135 | 0.001207 | −0.2593 | LCK |
| U70735 | 0.003096 | 0.002135 | 0.0002 | −0.20846 | MOV34-34KD |
| X79865 | 0.003096 | 0.002135 | 0.0141 | 0.418466 | MRPL12 |
| AI547258 | 0.003096 | 0.002135 | 0.001223 | 0.267951 | MT2A |
| L40387 | 0.003096 | 0.002135 | 0.00038 | 0.211973 | OASL |
| AB019517 | 0.003096 | 0.002135 | 0.023004 | 0.219453 | PKIG |
| M58459 | 0.003096 | 0.002135 | 0.001362 | 1.46854 | RPS4Y |
| X57348 | 0.003096 | 0.002135 | 0.004255 | 0.22047 | SFN |
| M74558 | 0.003096 | 0.002135 | 0.001205 | 0.219185 | SIL |
| U34044 | 0.003096 | 0.002135 | 0.000831 | −0.21289 | SPS |
| U49928 | 0.003096 | 0.002135 | 0.000886 | −0.31189 | TAB1 |
| X05839 | 0.003096 | 0.002135 | 0.008747 | 0.214552 | TGFB1 |
| U16296 | 0.003096 | 0.002135 | 0.006585 | −0.14857 | TIAM1 |
| U63127 | 0.003096 | 0.002135 | 0.000538 | −0.38925 | TIC |
| U03397 | 0.003096 | 0.002135 | 0.005156 | −0.34157 | TNFRSF9 |
| M21624 | 0.003096 | 0.002135 | 0.001748 | −0.51878 | TRD@ |
| D83198 | 0.003096 | 0.002135 | 0.028975 | −0.17519 | YF13H12 |
| HG960-HT960 | 0.003096 | 0.002135 | 0.003089 | 0.145701 | |
| HG4724-HT5166 | 0.003096 | 0.002135 | 0.002446 | −0.25728 | |
| D00654 | 0.003096 | 0.004342 | 9.79E−05 | −0.1819 | ACTG2 |
| U54645 | 0.013622 | 0.004342 | 0.004228 | −0.25281 | AK2 |
| M93405 | 0.003096 | 0.004342 | 0.020651 | 0.126156 | ALDH6A1 |
| U73960 | 0.003096 | 0.004342 | 0.002279 | 0.555806 | ARL4 |
| U26455 | 0.003096 | 0.004342 | 0.006562 | −0.53911 | ATM |
| M33519 | 0.003096 | 0.004342 | 0.011169 | −0.33327 | BAT3 |
| U90028 | 0.003096 | 0.004342 | 0.000396 | −0.24971 | BICD1 |
| AB002384 | 0.003096 | 0.004342 | 0.002855 | −0.46941 | C6orf32 |
| M74093 | 0.003096 | 0.004342 | 0.000763 | −0.33022 | CCNE1 |
| AA203246 | 0.003096 | 0.004342 | 0.007014 | −0.16607 | CDC2L5 |
| X66358 | 0.013622 | 0.004342 | 0.007122 | −0.1886 | CDKL1 |
| U30872 | 0.003096 | 0.004342 | 0.001715 | −0.164 | CENPF |
| AB020675 | 0.013622 | 0.004342 | 0.002913 | −0.25056 | CNTNAP2 |
| M13207 | 0.013622 | 0.004342 | 0.01388 | 0.122241 | CSF2 |
| AA173896 | 0.013622 | 0.004342 | 0.008401 | 0.305133 | CYB5-M |
| L78267 | 0.003096 | 0.004342 | 0.04708 | 0.103949 | D15S226E |
| AL080120 | 0.013622 | 0.004342 | 0.001834 | −0.12922 | DKFZP564O0423 |
| U13896 | 0.013622 | 0.004342 | 0.020482 | −0.10291 | DLG1 |
| AF034970 | 0.013622 | 0.004342 | 0.010371 | −0.10568 | DOK2 |
| D12686 | 0.013622 | 0.004342 | 0.003493 | 0.170378 | EIF4G1 |
| AB002386 | 0.003096 | 0.004342 | 0.000131 | −0.39255 | EZH1 |
| M15059 | 0.003096 | 0.004342 | 0.002497 | 0.2061 | FCER2 |
| W27545 | 0.013622 | 0.004342 | 0.004445 | 0.379682 | FLJ20259 |
| M84443 | 0.003096 | 0.004342 | 0.000101 | −0.27085 | GALK2 |
| AF029777 | 0.013622 | 0.004342 | 0.001427 | −0.22426 | GCN5L2 |
| D63876 | 0.013622 | 0.004342 | 0.002737 | 0.396946 | GGA3 |
| AB020645 | 0.003096 | 0.004342 | 0.003907 | −0.37377 | GLS |
| U77948 | 0.003096 | 0.004342 | 0.000818 | −0.35677 | GTF2I |
| AF035555 | 0.003096 | 0.004342 | 0.018388 | −0.17666 | HADH2 |
| AF055001 | 0.003096 | 0.004342 | 0.010744 | 0.724714 | HERPUD1 |
| D32129 | 0.003096 | 0.004342 | 0.005364 | −0.13287 | HLA-A |
| AF043586 | 0.003096 | 0.004342 | 0.001047 | −0.30021 | IGL@ |
| U53831 | 0.013622 | 0.004342 | 0.01853 | 0.488267 | IRF7 |
| AB002344 | 0.003096 | 0.004342 | 0.001658 | 0.705775 | KIAA0346 |
| AI677689 | 0.013622 | 0.004342 | 0.004375 | −0.1411 | KIAA0685 |
| AB023153 | 0.003096 | 0.004342 | 0.04282 | −0.39134 | KIAA0936 |
| AB023226 | 0.003096 | 0.004342 | 0.000111 | −0.71413 | KIAA1009 |
| AI148772 | 0.013622 | 0.004342 | 0.03739 | 0.532454 | KYNU |
| AB006780 | 0.003096 | 0.004342 | 0.010236 | 0.178362 | LGALS3 |
| AL050405 | 0.003096 | 0.004342 | 0.008144 | 0.311843 | LOC51634 |
| L35253 | 0.013622 | 0.004342 | 0.001324 | −0.46397 | MAPK14 |
| R93527 | 0.013622 | 0.004342 | 0.000372 | 0.264207 | MT1H |
| AF108145 | 0.003096 | 0.004342 | 0.001206 | −0.14877 | MYLE |
| M96980 | 0.013622 | 0.004342 | 0.002106 | −0.16409 | MYT1 |
| S76638 | 0.013622 | 0.004342 | 0.04529 | 0.171344 | NFKB2 |
| D88674 | 0.013622 | 0.004342 | 0.045232 | 0.346415 | OAZIN |
| AL050353 | 0.013622 | 0.004342 | 0.016071 | −0.11979 | OIP2 |
| AL080119 | 0.003096 | 0.004342 | 0.001961 | −0.40821 | PAI-RBP1 |
| X76770 | 0.013622 | 0.004342 | 0.005011 | −0.10613 | PAPOLA |
| D11466 | 0.003096 | 0.004342 | 0.009752 | 0.738127 | PIGA |
| W28299 | 0.003096 | 0.004342 | 0.001225 | −0.17755 | PINK1 |
| U83981 | 0.003096 | 0.004342 | 0.014327 | 0.28747 | PPP1R15A |
| X14968 | 0.013622 | 0.004342 | 0.004727 | 0.105215 | PRKAR2A |
| M55284 | 0.003096 | 0.004342 | 0.003435 | −0.17401 | PRKCH |
| M15036 | 0.003096 | 0.004342 | 0.010965 | −0.25119 | PROS1 |
| Y00638 | 0.003096 | 0.004342 | 0.004977 | −0.30956 | PTPRC |
| Y00815 | 0.003096 | 0.004342 | 0.015344 | 0.116938 | PTPRF |
| M38258 | 0.003096 | 0.004342 | 0.009252 | −0.14193 | RARG |
| AF025654 | 0.003096 | 0.004342 | 0.002302 | −0.39122 | RNGTT |
| M60724 | 0.013622 | 0.004342 | 0.004732 | −0.22065 | RPS6KB1 |
| AB006202 | 0.013622 | 0.004342 | 0.003028 | −0.18268 | SDHD |
| AA890010 | 0.003096 | 0.004342 | 0.00546 | −0.21285 | SEC22L1 |
| X62822 | 0.003096 | 0.004342 | 0.039707 | −0.21593 | SIAT1 |
| L41680 | 0.003096 | 0.004342 | 0.001771 | −0.16486 | SIAT8D |
| X15217 | 0.003096 | 0.004342 | 0.007377 | 0.149306 | SKIL |
| L13857 | 0.003096 | 0.004342 | 0.005721 | −0.11073 | SOS1 |
| U09564 | 0.003096 | 0.004342 | 0.001203 | −0.27717 | SRPK1 |
| Z75330 | 0.013622 | 0.004342 | 0.031796 | −0.11359 | STAG1 |
| X92762 | 0.003096 | 0.004342 | 0.001021 | −0.27946 | TAZ |
| AF064090 | 0.003096 | 0.004342 | 0.006206 | 0.303013 | TNFSF14 |
| U47634 | 0.003096 | 0.004342 | 0.0057 | 0.278205 | TUBB4 |
| L27071 | 0.003096 | 0.004342 | 0.000732 | −0.39906 | TXK |
| D78514 | 0.003096 | 0.004342 | 0.000681 | −0.2599 | UBE2G1 |
| AF085807 | 0.003096 | 0.004342 | 0.005801 | 0.124457 | UPK1A |
| U66561 | 0.003096 | 0.004342 | 0.002542 | 0.448044 | ZNF184 |
| X78925 | 0.013622 | 0.004342 | 0.001898 | 0.351929 | ZNF267 |
| HG2510-HT2606 | 0.013622 | 0.004342 | 0.007016 | 0.179499 | |
| W27419 | 0.003096 | 0.004342 | 0.006325 | 0.341787 | |
| AF054589 | 0.003096 | 0.004342 | 0.030568 | −0.50762 | |
| H98552 | 0.003096 | 0.004342 | 0.017185 | −0.1057 | |
| AI056697 | 0.003096 | 0.004342 | 0.000329 | −0.20147 | |
| X00351 | 0.003096 | 0.005207 | 0.001506 | −0.12928 | ACTB |
| AF006082 | 0.003096 | 0.005207 | 0.002797 | −0.34587 | ACTR2 |
| Y09443 | 0.003096 | 0.005207 | 0.002286 | −0.17646 | AGPS |
| U22961 | 0.003096 | 0.005207 | 0.003092 | 0.147932 | ALB |
| AF002163 | 0.003096 | 0.005207 | 0.002447 | −0.37588 | AP3D1 |
| D87461 | 0.003096 | 0.005207 | 0.004809 | −0.26338 | BCL2L2 |
| AF013759 | 0.003096 | 0.005207 | 0.004946 | −0.18574 | CALU |
| L22005 | 0.003096 | 0.005207 | 0.006442 | 0.131869 | CDC34 |
| AL109689 | 0.003096 | 0.005207 | 0.013291 | −0.24945 | CGI-142 |
| U91543 | 0.003096 | 0.005207 | 0.014143 | −0.25258 | CHD3 |
| X82153 | 0.003096 | 0.005207 | 0.013882 | −0.31742 | CTSK |
| AJ001687 | 0.003096 | 0.005207 | 0.000224 | −0.64837 | D12S2489E |
| M13149 | 0.003096 | 0.005207 | 0.008717 | −0.13824 | HRG |
| Y10313 | 0.003096 | 0.005207 | 0.006846 | 0.464769 | IFRD1 |
| D63485 | 0.003096 | 0.005207 | 0.000985 | −0.31599 | IKKE |
| D87077 | 0.003096 | 0.005207 | 0.043072 | −0.21138 | KIAA0240 |
| AB007864 | 0.003096 | 0.005207 | 0.001569 | 0.256672 | KIAA0404 |
| X75346 | 0.003096 | 0.005207 | 0.001841 | 0.331699 | MAPKALPK2 |
| L07648 | 0.003096 | 0.005207 | 0.010594 | 0.226817 | MXI1 |
| AB028993 | 0.003096 | 0.005207 | 0.0247 | 0.133216 | NLGN1 |
| D45333 | 0.003096 | 0.005207 | 0.002104 | 0.302454 | PFDN1 |
| M65254 | 0.003096 | 0.005207 | 0.002619 | 0.262897 | PPP2R1B |
| M86852 | 0.003096 | 0.005207 | 0.004274 | 0.172251 | PXMP3 |
| X97795 | 0.003096 | 0.005207 | 0.021131 | −0.18349 | RAD54L |
| U14970 | 0.003096 | 0.005207 | 0.001894 | −0.1353 | RPS5 |
| X74570 | 0.003096 | 0.005207 | 0.00345 | 0.210049 | SIAT4C |
| X98248 | 0.003096 | 0.005207 | 0.010403 | −0.50617 | SORT1 |
| U17714 | 0.003096 | 0.005207 | 0.002081 | −0.19372 | ST13 |
| W28869 | 0.003096 | 0.005207 | 0.001369 | −0.38498 | TEGT |
| M12125 | 0.003096 | 0.005207 | 0.000178 | −0.09929 | TPM2 |
| L27071 | 0.003096 | 0.005207 | 0.003834 | −0.36074 | TXK |
| M60614 | 0.003096 | 0.005207 | 0.001757 | −0.25283 | WIT-1 |
| HG4074-HT4344 | 0.003096 | 0.005207 | 0.004175 | 0.589048 | |
| AL031846 | 0.003096 | 0.005207 | 0.004012 | −0.42132 | |
| HG1980-HT2023 | 0.003096 | 0.005207 | 0.002314 | 0.711234 | |
| AF022853 | 0.047678 | 0.006683 | 0.002056 | −0.30792 | ABCC1 |
| X02994 | 0.047678 | 0.006683 | 0.036598 | −0.12393 | ADA |
| D25304 | 0.047678 | 0.006683 | 0.002258 | −0.44746 | ARHGEF6 |
| M23115 | 0.047678 | 0.006683 | 0.016518 | −0.1243 | ATP2A2 |
| U87408 | 0.047678 | 0.006683 | 0.008628 | −0.33961 | B1 |
| AA135683 | 0.047678 | 0.006683 | 0.010045 | 0.6329 | BASP1 |
| M22491 | 0.047678 | 0.006683 | 0.020141 | −0.10386 | BMP3 |
| M28170 | 0.047678 | 0.006683 | 0.014303 | 0.280093 | CD19 |
| M16336 | 0.047678 | 0.006683 | 0.011755 | −0.19993 | CD2 |
| U37022 | 0.047678 | 0.006683 | 0.028135 | −0.06885 | CDK4 |
| U66469 | 0.047678 | 0.006683 | 0.004123 | 0.616896 | CGR19 |
| AI037867 | 0.047678 | 0.006683 | 0.009634 | −0.11973 | CKTSF1B1 |
| J03071 | 0.047678 | 0.006683 | 0.011153 | −0.23776 | CSH2 |
| M55265 | 0.047678 | 0.006683 | 0.01278 | −0.1479 | CSNK2A1 |
| M33317 | 0.047678 | 0.006683 | 0.014832 | −0.17753 | CYP2A7 |
| U37143 | 0.013622 | 0.006683 | 0.001908 | 0.171138 | CYP2J2 |
| AL049942 | 0.013622 | 0.006683 | 0.00076 | −0.20245 | DKFZP564F1422 |
| AL050015 | 0.013622 | 0.006683 | 0.008524 | −0.13959 | DKFZP564O243 |
| L35594 | 0.013622 | 0.006683 | 0.002806 | 0.216985 | ENPP2 |
| J03796 | 0.047678 | 0.006683 | 0.002596 | −0.28198 | EPB41 |
| AC002398 | 0.013622 | 0.006683 | 0.003226 | −0.27062 | F25965 |
| X15376 | 0.013622 | 0.006683 | 0.014388 | −0.15607 | GABRG2 |
| M90656 | 0.047678 | 0.006683 | 0.006961 | −0.15968 | GCLC |
| AF062006 | 0.013622 | 0.006683 | 0.001442 | 0.200117 | GPR49 |
| X61755 | 0.013622 | 0.006683 | 0.000491 | −0.19331 | HOXC5 |
| D21851 | 0.047678 | 0.006683 | 0.017915 | 0.153927 | KIAA0028 |
| AB007976 | 0.047678 | 0.006683 | 0.032427 | 0.228873 | KIAA0507 |
| AI871396 | 0.047678 | 0.006683 | 0.001957 | −0.48312 | KIAA0557 |
| AB020660 | 0.047678 | 0.006683 | 0.001858 | −0.27616 | KIAA0853 |
| X93595 | 0.047678 | 0.006683 | 0.026214 | 0.245064 | KIR3DL2 |
| AB002405 | 0.047678 | 0.006683 | 0.003681 | −0.19481 | LAK-4P |
| X07228 | 0.047678 | 0.006683 | 0.046458 | 0.113484 | LIPC |
| U50529 | 0.047678 | 0.006683 | 0.001977 | 0.310499 | LOC88523 |
| AF040963 | 0.047678 | 0.006683 | 0.012629 | 0.148739 | MAD4 |
| U59423 | 0.047678 | 0.006683 | 0.01126 | −0.1341 | MADH1 |
| U85430 | 0.013622 | 0.006683 | 0.000224 | −0.41454 | NFATC3 |
| X80878 | 0.047678 | 0.006683 | 0.010021 | −0.16096 | NFRKB |
| AF005043 | 0.013622 | 0.006683 | 0.00078 | −0.15296 | PARG |
| D49818 | 0.047678 | 0.006683 | 0.021976 | −0.10631 | PFKFB4 |
| M28393 | 0.013622 | 0.006683 | 0.003241 | −0.16419 | PRF1 |
| Y00062 | 0.047678 | 0.006683 | 0.011868 | −0.2415 | PTPRC |
| L07758 | 0.047678 | 0.006683 | 0.008943 | 0.201883 | PWP1 |
| U57094 | 0.047678 | 0.006683 | 0.014944 | −0.31108 | RAB27A |
| M35416 | 0.013622 | 0.006683 | 0.002789 | −0.41233 | RALB |
| X75042 | 0.047678 | 0.006683 | 0.003614 | 0.659166 | REL |
| AF038250 | 0.047678 | 0.006683 | 0.004198 | 0.395171 | SFRS3 |
| L27213 | 0.013622 | 0.006683 | 0.001014 | −0.13065 | SLC4A3 |
| Y09568 | 0.047678 | 0.006683 | 0.005799 | −0.3407 | SNAP23 |
| AA205857 | 0.013622 | 0.006683 | 0.00048 | 0.27495 | SNRPD3 |
| U07794 | 0.047678 | 0.006683 | 0.007632 | −0.20733 | TXK |
| J05428 | 0.013622 | 0.006683 | 0.00573 | −0.08342 | UGT2B7 |
| U09848 | 0.047678 | 0.006683 | 0.011538 | −0.26846 | ZNF36 |
| J00287 | 0.047678 | 0.006683 | 0.000953 | −0.28381 | |
| AB007882 | 0.013622 | 0.009369 | 0.006034 | −0.17275 | ADCY6 |
| AF072810 | 0.013622 | 0.009369 | 0.016137 | −0.32509 | BAZ1B |
| AB004066 | 0.013622 | 0.009369 | 0.00789 | 0.494455 | BHLHB2 |
| U37547 | 0.013622 | 0.009369 | 0.00478 | 0.544595 | BIRC2 |
| AB024704 | 0.013622 | 0.009369 | 0.010893 | −0.11914 | C20orf1 |
| AC004084 | 0.013622 | 0.009369 | 0.005491 | −0.17437 | CAPRI |
| L12691 | 0.013622 | 0.009369 | 0.018291 | −0.18848 | DEFA3 |
| L19161 | 0.013622 | 0.009369 | 0.001645 | −0.27253 | EIF2S3 |
| M82882 | 0.013622 | 0.009369 | 0.003966 | 0.478256 | ELF1 |
| X81625 | 0.013622 | 0.009369 | 0.003918 | 0.762544 | ETF1 |
| M15059 | 0.013622 | 0.009369 | 0.035106 | 0.313247 | FCER2 |
| AA284298 | 0.013622 | 0.009369 | 0.028745 | −0.12535 | FLJ22269 |
| U13044 | 0.013622 | 0.009369 | 0.017986 | −0.32813 | GABPA |
| Z12173 | 0.013622 | 0.009369 | 0.001297 | −0.32703 | GNS |
| U06631 | 0.013622 | 0.009369 | 0.003506 | −0.37935 | H326 |
| X75315 | 0.013622 | 0.009369 | 0.002959 | 1.06191 | HSRNASEB |
| AF064084 | 0.013622 | 0.009369 | 0.017927 | −0.10308 | ICMT |
| AB002330 | 0.013622 | 0.009369 | 0.048512 | −0.09076 | KIAA0332 |
| AB014569 | 0.013622 | 0.009369 | 0.010011 | 0.709572 | KIAA0669 |
| AI970189 | 0.013622 | 0.009369 | 0.001778 | 0.569801 | KIAA0997 |
| AB028960 | 0.013622 | 0.009369 | 0.001825 | −0.15403 | KIAA1037 |
| AJ005273 | 0.013622 | 0.009369 | 0.001449 | 0.379277 | KIN |
| L00352 | 0.013622 | 0.009369 | 0.004231 | 0.554465 | LDLR |
| X54304 | 0.013622 | 0.009369 | 0.000863 | −0.19567 | MLCB |
| AI693193 | 0.013622 | 0.009369 | 0.023978 | −0.25831 | MTX1 |
| AF047487 | 0.013622 | 0.009369 | 0.001312 | −0.33746 | NCK2 |
| AF037448 | 0.013622 | 0.009369 | 0.00743 | 0.204106 | NSAP1 |
| AF000152 | 0.013622 | 0.009369 | 0.025216 | −0.34592 | OS4 |
| U02882 | 0.013622 | 0.009369 | 0.017536 | 0.892321 | PDE4D |
| X89416 | 0.013622 | 0.009369 | 0.005129 | −0.1405 | PPP5C |
| U27516 | 0.013622 | 0.009369 | 0.004164 | −0.17553 | RAD52 |
| D23660 | 0.013622 | 0.009369 | 0.01215 | 0.149327 | RPL4 |
| AB016247 | 0.013622 | 0.009369 | 0.019879 | 0.416634 | SC5DL |
| U44754 | 0.013622 | 0.009369 | 0.005273 | 0.158396 | SNAPC1 |
| AI660929 | 0.013622 | 0.009369 | 0.000698 | −0.15764 | T1A-2 |
| X01060 | 0.013622 | 0.009369 | 0.005079 | 0.27369 | TFRC |
| J02973 | 0.013622 | 0.009369 | 0.006825 | 0.835338 | THBD |
| L41690 | 0.013622 | 0.009369 | 0.020209 | −0.32814 | TRADD |
| X89066 | 0.013622 | 0.009369 | 0.000396 | −0.2226 | TRPC1 |
| AB024327 | 0.013622 | 0.009369 | 0.031 | 0.260875 | UNRIP |
| AF033199 | 0.013622 | 0.009369 | 0.02842 | −0.1806 | ZNF204 |
| AL080123 | 0.013622 | 0.009369 | 0.018447 | 0.215445 | ZNF23 |
| AB007885 | 0.013622 | 0.009369 | 0.025803 | −0.22701 | ZNF262 |
| U40462 | 0.013622 | 0.009369 | 0.004101 | −0.29722 | ZNFN1A1 |
| HG3477-HT3670 | 0.013622 | 0.009369 | 0.00042 | −0.2367 | |
| L42324 | 0.013622 | 0.009369 | 0.015195 | 0.283048 | GPR18 |
| AA975427 | 0.013622 | 0.009369 | 0.002377 | −0.26992 | |
| AL049957 | 0.013622 | 0.009369 | 0.007809 | 0.133451 | |
| AL022398 | 0.013622 | 0.009369 | 0.017529 | −0.48579 | |
| HG2689-HT2785 | 0.013622 | 0.009369 | 0.029818 | 0.202486 | |
| AF034373 | 0.013622 | 0.014679 | 0.00591 | −0.26511 | A2LP |
| X83467 | 0.013622 | 0.014679 | 0.006111 | −0.25837 | ABCD3 |
| U41766 | 0.047678 | 0.014679 | 0.014363 | 0.473526 | ADAM9 |
| D67031 | 0.013622 | 0.014679 | 0.007826 | −0.4645 | ADD3 |
| U84011 | 0.013622 | 0.014679 | 0.012995 | −0.2499 | AGL |
| M74088 | 0.013622 | 0.014679 | 0.038601 | −0.16952 | APC |
| U67092 | 0.047678 | 0.014679 | 0.047381 | −0.10935 | ATM |
| AI033692 | 0.047678 | 0.014679 | 0.010056 | −0.2417 | BCRP1 |
| X92106 | 0.013622 | 0.014679 | 0.001377 | −0.33994 | BLMH |
| Z22535 | 0.047678 | 0.014679 | 0.021613 | −0.09832 | BMPR1A |
| X79067 | 0.047678 | 0.014679 | 0.0381 | 0.14954 | BRF1 |
| X86098 | 0.013622 | 0.014679 | 0.000807 | −0.34829 | BS69 |
| U72649 | 0.047678 | 0.014679 | 0.029157 | 0.227444 | BTG2 |
| X95592 | 0.047678 | 0.014679 | 0.014127 | 0.201273 | C1D |
| D78586 | 0.047678 | 0.014679 | 0.028845 | −0.05709 | CAD |
| D30742 | 0.047678 | 0.014679 | 0.028215 | 0.180381 | CAMK4 |
| AF035582 | 0.047678 | 0.014679 | 0.002042 | 0.530946 | CASK |
| U60521 | 0.047678 | 0.014679 | 0.002353 | 0.552837 | CASP9 |
| AL035079 | 0.013622 | 0.014679 | 0.00063 | −0.78567 | CAT |
| AF094481 | 0.047678 | 0.014679 | 0.024569 | 0.149973 | CGGBP1 |
| X83378 | 0.013622 | 0.014679 | 0.025603 | 0.133485 | CLCN6 |
| AB002332 | 0.047678 | 0.014679 | 0.003136 | −0.20009 | CLOCK |
| D13146 | 0.013622 | 0.014679 | 0.017967 | −0.13385 | CNP |
| S80864 | 0.013622 | 0.014679 | 0.011106 | −0.33164 | CYCL |
| D17530 | 0.047678 | 0.014679 | 0.005519 | −0.15234 | DBN1 |
| U87947 | 0.047678 | 0.014679 | 0.011279 | 0.222382 | EMP3 |
| M60459 | 0.047678 | 0.014679 | 0.010174 | −0.10156 | EPOR |
| AB018247 | 0.013622 | 0.014679 | 0.000348 | 0.423577 | FE65L2 |
| AB028973 | 0.013622 | 0.014679 | 0.046458 | −0.12088 | FLJ10883 |
| AL080172 | 0.047678 | 0.014679 | 0.02693 | −0.063 | FLJ21919 |
| AF032886 | 0.047678 | 0.014679 | 0.009814 | 0.232307 | FOXO3A |
| U00928 | 0.047678 | 0.014679 | 0.01307 | −0.0915 | FUS |
| M14660 | 0.047678 | 0.014679 | 0.011038 | 0.732462 | FUT10 |
| AI935146 | 0.047678 | 0.014679 | 0.044653 | 0.246267 | GALNT3 |
| U28811 | 0.047678 | 0.014679 | 0.007572 | −0.21558 | GLG1 |
| AF001903 | 0.013622 | 0.014679 | 0.001957 | −0.28636 | HADHSC |
| Y09306 | 0.047678 | 0.014679 | 0.045083 | −0.08024 | HIPK3 |
| AL022723 | 0.047678 | 0.014679 | 0.041021 | 0.165267 | HLA-G |
| M80469 | 0.013622 | 0.014679 | 0.037453 | −0.12099 | HLA-J |
| M16937 | 0.013622 | 0.014679 | 0.002262 | −0.13536 | HOXB7 |
| X98307 | 0.013622 | 0.014679 | 0.011852 | −0.0908 | HSHUR7SEQ |
| HG2855-HT2995 | 0.047678 | 0.014679 | 0.030595 | 0.16813 | HSP70 |
| X87949 | 0.047678 | 0.014679 | 0.028569 | 0.296273 | HSPA5 |
| W68830 | 0.013622 | 0.014679 | 0.007971 | −0.22855 | HSPC022 |
| D49410 | 0.047678 | 0.014679 | 0.040369 | 0.153358 | HUMIL3RA12 |
| AL049470 | 0.013622 | 0.014679 | 0.010492 | 0.283688 | HYPB |
| Y10659 | 0.047678 | 0.014679 | 0.024205 | −0.1217 | IL13RA1 |
| X52015 | 0.047678 | 0.014679 | 0.006637 | 0.417081 | IL1RN |
| AF047492 | 0.047678 | 0.014679 | 0.002557 | 0.25738 | IMPG1 |
| U96919 | 0.013622 | 0.014679 | 0.003221 | −0.19947 | INPP4A |
| U12897 | 0.013622 | 0.014679 | 0.002496 | −0.15016 | IPW |
| S62539 | 0.013622 | 0.014679 | 0.012982 | −0.20615 | IRS1 |
| AF029778 | 0.047678 | 0.014679 | 0.018006 | −0.14486 | JAG2 |
| W25934 | 0.047678 | 0.014679 | 0.016925 | 0.363279 | JTV1 |
| X56681 | 0.047678 | 0.014679 | 0.004935 | 0.713663 | JUND |
| M64934 | 0.047678 | 0.014679 | 0.003162 | −0.1823 | KEL |
| D86975 | 0.047678 | 0.014679 | 0.048475 | 0.163408 | KIAA0222 |
| AB020701 | 0.013622 | 0.014679 | 0.013946 | 0.283086 | KIAA0894 |
| AB023141 | 0.047678 | 0.014679 | 0.017326 | −0.33543 | KIAA0924 |
| AB023148 | 0.013622 | 0.014679 | 0.016218 | −0.27496 | KIAA0931 |
| AB023227 | 0.047678 | 0.014679 | 0.043542 | 0.316063 | KIAA1010 |
| AB028963 | 0.047678 | 0.014679 | 0.039194 | −0.12296 | KIAA1040 |
| AL080188 | 0.047678 | 0.014679 | 0.016745 | −0.10387 | KIAA1775 |
| AJ224162 | 0.013622 | 0.014679 | 0.002225 | −0.24337 | LAS |
| L25931 | 0.013622 | 0.014679 | 0.00482 | −0.2367 | LBR |
| AC004410 | 0.047678 | 0.014679 | 0.017457 | 0.210096 | LOC56928 |
| AB009462 | 0.047678 | 0.014679 | 0.012892 | 0.131673 | LRP3 |
| AF077820 | 0.013622 | 0.014679 | 0.003095 | −0.40005 | LRP5 |
| X59408 | 0.047678 | 0.014679 | 0.018321 | −0.3029 | MCP |
| L13773 | 0.013622 | 0.014679 | 0.002741 | −0.18297 | MLLT2 |
| X82209 | 0.047678 | 0.014679 | 0.010828 | 0.178564 | MN1 |
| X96401 | 0.013622 | 0.014679 | 0.001643 | 0.317165 | MNT |
| M30818 | 0.047678 | 0.014679 | 0.032832 | 0.292682 | MX2 |
| V00568 | 0.013622 | 0.014679 | 0.008535 | −0.58978 | MYC |
| D50692 | 0.013622 | 0.014679 | 0.043374 | −0.20783 | MYCBP |
| AB007191 | 0.013622 | 0.014679 | 0.022026 | −0.18098 | MYCBP |
| X17576 | 0.013622 | 0.014679 | 0.001641 | −0.26027 | NCK1 |
| X61498 | 0.013622 | 0.014679 | 0.006234 | 0.307667 | NFKB2 |
| AF052093 | 0.047678 | 0.014679 | 0.001318 | −0.31976 | NJMU-R1 |
| X00737 | 0.047678 | 0.014679 | 0.037385 | 0.219194 | NP |
| U02020 | 0.047678 | 0.014679 | 0.014866 | 0.650286 | PBEF |
| X66362 | 0.047678 | 0.014679 | 0.006159 | 0.137944 | PCTK3 |
| AF026086 | 0.047678 | 0.014679 | 0.006555 | −0.18222 | PEX1 |
| L25441 | 0.047678 | 0.014679 | 0.011907 | 0.146471 | PGGT1B |
| AL021366 | 0.013622 | 0.014679 | 0.002775 | 0.425217 | PHF1 |
| D85418 | 0.013622 | 0.014679 | 0.004449 | −0.31688 | PIGC |
| D30037 | 0.047678 | 0.014679 | 0.001579 | −0.21226 | PITPNB |
| AB006746 | 0.047678 | 0.014679 | 0.0356 | 0.189986 | PLSCR1 |
| AF054182 | 0.013622 | 0.014679 | 0.002098 | −0.54761 | PMPCB |
| S87759 | 0.013622 | 0.014679 | 0.007522 | 0.39052 | PPM1A |
| M13057 | 0.047678 | 0.014679 | 0.032523 | −0.19317 | PRH1 |
| M64992 | 0.047678 | 0.014679 | 0.047326 | 0.178696 | PSMA1 |
| X58288 | 0.047678 | 0.014679 | 0.002633 | 0.409542 | PTPRM |
| AD000092 | 0.047678 | 0.014679 | 0.028359 | 0.137917 | RAD23A |
| U79716 | 0.013622 | 0.014679 | 0.003409 | 0.195389 | RELN |
| U69198 | 0.047678 | 0.014679 | 0.048001 | 0.085316 | RFNG |
| AF117829 | 0.047678 | 0.014679 | 0.003668 | 0.377251 | RIPK2 |
| AF039029 | 0.047678 | 0.014679 | 0.002146 | −0.28622 | RNUT1 |
| AW021542 | 0.013622 | 0.014679 | 0.000677 | −0.29232 | SAP18 |
| U64197 | 0.047678 | 0.014679 | 0.021124 | 0.220476 | SCYA20 |
| AB023136 | 0.013622 | 0.014679 | 0.00288 | −0.10963 | SEC15B |
| AF055006 | 0.013622 | 0.014679 | 0.011241 | 0.238955 | SEC6 |
| Z46606 | 0.047678 | 0.014679 | 0.005778 | −0.1566 | SMARCA3 |
| L25270 | 0.047678 | 0.014679 | 0.002401 | −0.15644 | SMCX |
| M60618 | 0.013622 | 0.014679 | 0.006316 | 0.235838 | SP100 |
| AI739308 | 0.013622 | 0.014679 | 0.001861 | −0.57419 | SRP46 |
| U52960 | 0.047678 | 0.014679 | 0.02599 | 0.429086 | SURB7 |
| D50863 | 0.013622 | 0.014679 | 0.006582 | −0.13005 | TESK1 |
| D64015 | 0.013622 | 0.014679 | 0.007587 | −0.3629 | TIAL1 |
| AB001523 | 0.047678 | 0.014679 | 0.027565 | 0.164838 | TMEM1 |
| L21715 | 0.013622 | 0.014679 | 0.000862 | 0.309808 | TNNI2 |
| AF045583 | 0.047678 | 0.014679 | 0.043887 | −0.16757 | TULP3 |
| AJ001340 | 0.013622 | 0.014679 | 0.002396 | −0.17031 | U3-55K |
| AB015344 | 0.013622 | 0.014679 | 0.008107 | −0.31161 | UBQLN2 |
| J03824 | 0.013622 | 0.014679 | 0.005864 | −0.18849 | UROS |
| AF022789 | 0.047678 | 0.014679 | 0.006582 | 0.309267 | USP12 |
| U48801 | 0.013622 | 0.014679 | 0.003849 | −0.17743 | VEGFB |
| HG544-HT544 | 0.047678 | 0.014679 | 0.010549 | 0.454218 | |
| S66666 | 0.013622 | 0.014679 | 0.003364 | −0.14303 | |
| AI687419 | 0.047678 | 0.014679 | 0.039394 | −0.3657 | |
| W28800 | 0.047678 | 0.014679 | 0.004582 | 0.270831 | |
| AL080111 | 0.013622 | 0.014679 | 0.001378 | −0.36029 | |
| AF070536 | 0.047678 | 0.014679 | 0.006685 | 0.199364 | |
| AF070633 | 0.047678 | 0.014679 | 0.010142 | −0.1635 | |
| AF054998 | 0.013622 | 0.014679 | 0.007913 | −0.21157 | |
| HG3725-HT3981 | 0.047678 | 0.014679 | 0.027792 | −0.11953 | |
| HG1614-HT1614 | 0.013622 | 0.014679 | 0.006999 | −0.45233 | |
| M22324 | 0.013622 | 0.01669 | 0.009982 | 0.283293 | ANPEP |
| AC005955 | 0.013622 | 0.01669 | 0.004346 | 0.137324 | CEACAM4 |
| S68134 | 0.013622 | 0.01669 | 0.005372 | 1.92718 | CREM |
| S68271 | 0.013622 | 0.01669 | 0.009154 | 1.49785 | CREM |
| M24069 | 0.013622 | 0.01669 | 0.003022 | 0.249971 | CSDA |
| AF000984 | 0.013622 | 0.01669 | 0.004295 | 0.46432 | DBY |
| AF055917 | 0.013622 | 0.01669 | 0.015434 | 0.102855 | F2RL3 |
| U27333 | 0.013622 | 0.01669 | 0.012662 | 0.136047 | FUT6 |
| X89887 | 0.013622 | 0.01669 | 0.009728 | 0.152829 | HIRA |
| L42243 | 0.013622 | 0.01669 | 0.002638 | 0.218644 | IFNAR2 |
| AI950382 | 0.013622 | 0.01669 | 0.00744 | 0.601631 | KIAA0585 |
| AI950382 | 0.013622 | 0.01669 | 0.002126 | 0.519735 | KIAA0585 |
| U17760 | 0.013622 | 0.01669 | 0.044392 | 0.431131 | LAMB3 |
| L48692 | 0.013622 | 0.01669 | 0.041233 | 0.63409 | LOC56902 |
| X94232 | 0.013622 | 0.01669 | 0.016402 | 0.326694 | MAPRE2 |
| AA037278 | 0.013622 | 0.01669 | 0.01607 | 0.119411 | MGC10882 |
| L13740 | 0.013622 | 0.01669 | 0.007795 | 0.355688 | NR4A1 |
| U12767 | 0.013622 | 0.01669 | 0.011648 | 1.30268 | NR4A3 |
| D78579 | 0.013622 | 0.01669 | 0.005896 | 1.11766 | NR4A3 |
| X17042 | 0.013622 | 0.01669 | 0.015594 | 0.239796 | PRG1 |
| U48296 | 0.013622 | 0.01669 | 0.003124 | 0.864101 | PTP4A1 |
| M83221 | 0.013622 | 0.01669 | 0.012321 | 0.192956 | RELB |
| AF107463 | 0.013622 | 0.01669 | 0.009662 | 0.419254 | SPF30 |
| L47276 | 0.013622 | 0.01669 | 0.004673 | 0.194449 | TOP2A |
| X00734 | 0.013622 | 0.01669 | 0.010039 | 0.347307 | TUBB5 |
| X51521 | 0.013622 | 0.01669 | 0.010303 | 0.60161 | VIL2 |
| S54641 | 0.013622 | 0.01669 | 0.008483 | 0.183207 | ZNF124 |
| M91029 | 0.013622 | 0.022759 | 0.010686 | 0.450612 | AMPD2 |
| AB021638 | 0.136189 | 0.022759 | 0.024881 | −0.1126 | APBA3 |
| AL120559 | 0.013622 | 0.022759 | 0.004505 | 0.577915 | ARPP-19 |
| AF039656 | 0.013622 | 0.022759 | 0.006991 | 0.68481 | BASP1 |
| AB020623 | 0.047678 | 0.022759 | 0.009696 | 0.418826 | BCAS2 |
| X60201 | 0.013622 | 0.022759 | 0.011758 | −0.1576 | BDNF |
| U56637 | 0.047678 | 0.022759 | 0.008899 | −0.28102 | CAPZA1 |
| AW043690 | 0.047678 | 0.022759 | 0.031971 | 0.134862 | CCK |
| D13627 | 0.047678 | 0.022759 | 0.019298 | 0.203913 | CCT8 |
| U56998 | 0.013622 | 0.022759 | 0.024403 | 0.442545 | CNK |
| U71267 | 0.047678 | 0.022759 | 0.007233 | −0.13426 | CNOT4 |
| F27891 | 0.047678 | 0.022759 | 0.02847 | 0.119514 | COX6A2 |
| U78524 | 0.013622 | 0.022759 | 0.002554 | 0.353034 | DDXBP1 |
| AF043733 | 0.047678 | 0.022759 | 0.005645 | 0.22771 | DEDD |
| X64229 | 0.013622 | 0.022759 | 0.013033 | −0.20244 | DEK |
| AL050284 | 0.047678 | 0.022759 | 0.002819 | 0.232244 | DKFZP586M1019 |
| L05147 | 0.013622 | 0.022759 | 0.021168 | 0.111752 | DUSP3 |
| U15642 | 0.013622 | 0.022759 | 0.013339 | 0.474421 | E2F5 |
| U31556 | 0.047678 | 0.022759 | 0.011303 | 0.335871 | E2F5 |
| AC004262 | 0.047678 | 0.022759 | 0.004968 | −0.25642 | EMR2 |
| AA181196 | 0.047678 | 0.022759 | 0.009459 | −0.10534 | FLJ11712 |
| U74612 | 0.013622 | 0.022759 | 0.014802 | −0.18783 | FOXM1 |
| W28281 | 0.013622 | 0.022759 | 0.011042 | 0.813742 | GABARAPL1 |
| AI183417 | 0.013622 | 0.022759 | 0.011016 | 0.117979 | GABPB1 |
| L13720 | 0.013622 | 0.022759 | 0.014471 | −0.1601 | GAS6 |
| X15722 | 0.013622 | 0.022759 | 0.029451 | −0.19175 | GSR |
| Y07595 | 0.013622 | 0.022759 | 0.003113 | −0.20996 | GTF2H4 |
| L43821 | 0.047678 | 0.022759 | 0.005863 | −0.20401 | HEF1 |
| L10379 | 0.013622 | 0.022759 | 0.02006 | −0.15961 | HRIHFB2206 |
| X99209 | 0.013622 | 0.022759 | 0.021333 | −0.14942 | HRMT1L1 |
| X77956 | 0.013622 | 0.022759 | 0.009598 | 0.591031 | ID1 |
| AL021707 | 0.013622 | 0.022759 | 0.004161 | 1.79061 | KIAA0063 |
| AB007896 | 0.013622 | 0.022759 | 0.006273 | −0.41247 | KIAA0436 |
| AB014528 | 0.047678 | 0.022759 | 0.001992 | −0.31837 | KIAA0628 |
| AB014607 | 0.013622 | 0.022759 | 0.000764 | −0.15753 | KIAA0707 |
| AB018290 | 0.013622 | 0.022759 | 0.034506 | −0.28703 | KIAA0747 |
| AB018337 | 0.013622 | 0.022759 | 0.008466 | −0.41118 | KIAA0794 |
| AB023161 | 0.013622 | 0.022759 | 0.018461 | −0.15095 | KIAA0944 |
| AB023202 | 0.013622 | 0.022759 | 0.005879 | −0.19156 | KIAA0985 |
| U80743 | 0.013622 | 0.022759 | 0.000544 | −0.30322 | KIAA1498 |
| X13794 | 0.047678 | 0.022759 | 0.018671 | −0.12764 | LDHB |
| Z34975 | 0.013622 | 0.022759 | 0.012256 | −0.29089 | LDLC |
| AI341656 | 0.047678 | 0.022759 | 0.021482 | −0.26002 | LIM |
| X87342 | 0.013622 | 0.022759 | 0.006652 | −0.23382 | LLGL2 |
| U29671 | 0.047678 | 0.022759 | 0.001133 | −0.2617 | MAP3K1 |
| Z14138 | 0.013622 | 0.022759 | 0.00408 | 0.81232 | MAP3K8 |
| AI743606 | 0.013622 | 0.022759 | 0.00269 | −0.19764 | MEL |
| AF052183 | 0.013622 | 0.022759 | 0.002151 | −0.19631 | MGC2722 |
| AL050356 | 0.013622 | 0.022759 | 0.002743 | −0.42417 | MINPP1 |
| AF041081 | 0.013622 | 0.022759 | 0.019282 | −0.21627 | MN7 |
| U59302 | 0.013622 | 0.022759 | 0.003859 | 0.280175 | NCOA1 |
| W28360 | 0.013622 | 0.022759 | 0.016633 | 0.272057 | NCUBE1 |
| U97198 | 0.013622 | 0.022759 | 0.001352 | −0.20163 | NLP_1 |
| AA194159 | 0.013622 | 0.022759 | 0.004614 | −0.40044 | PEX10 |
| U38964 | 0.013622 | 0.022759 | 0.004912 | −0.23793 | PMS2L8 |
| D38498 | 0.013622 | 0.022759 | 0.003965 | −0.58306 | PMS2L9 |
| AA996066 | 0.013622 | 0.022759 | 0.003514 | −0.21994 | PMS2L9 |
| AB029028 | 0.013622 | 0.022759 | 0.027753 | −0.29778 | RAP140 |
| AA402524 | 0.047678 | 0.022759 | 0.005359 | −0.11564 | RBM9 |
| U79745 | 0.013622 | 0.022759 | 0.00409 | 0.777629 | SLC16A6 |
| X98332 | 0.013622 | 0.022759 | 0.002282 | −0.20078 | SLC22A1 |
| D42045 | 0.013622 | 0.022759 | 0.006867 | −0.19726 | SNM1 |
| M76231 | 0.013622 | 0.022759 | 0.009942 | 0.13899 | SPR |
| U76366 | 0.013622 | 0.022759 | 0.015416 | −0.09378 | TCOF1 |
| U09087 | 0.013622 | 0.022759 | 0.00607 | −0.26017 | TMPO |
| AF049140 | 0.047678 | 0.022759 | 0.011115 | −0.21894 | UBE2V2 |
| AF038962 | 0.047678 | 0.022759 | 0.007219 | −0.44337 | VDAC3 |
| D84145 | 0.013622 | 0.022759 | 0.002923 | 0.574155 | WS-3 |
| Y09723 | 0.047678 | 0.022759 | 0.001292 | 0.234149 | ZNF151 |
| AL049991 | 0.013622 | 0.022759 | 0.007094 | 0.245237 | |
| AL050148 | 0.013622 | 0.022759 | 0.013128 | −0.26398 | |
| AI014538 | 0.013622 | 0.022759 | 0.003507 | −0.15278 | |
| AI732885 | 0.047678 | 0.022759 | 0.043886 | −0.10293 | |
| U14573 | 0.013622 | 0.022759 | 0.04259 | −0.11614 | |
| U82987 | 0.013622 | 0.024606 | 0.00343 | −0.17272 | BBC3 |
| L12168 | 0.013622 | 0.024606 | 0.007944 | −0.22028 | CAP |
| V00571 | 0.013622 | 0.024606 | 0.005873 | 0.132015 | CRH |
| AL022398 | 0.013622 | 0.024606 | 0.015005 | −0.44535 | DJ434O14.3 |
| AL080081 | 0.013622 | 0.024606 | 0.007659 | 0.548836 | DNAJB9 |
| X85116 | 0.013622 | 0.024606 | 0.039531 | −0.24601 | EPB72 |
| AJ007669 | 0.013622 | 0.024606 | 0.019404 | −0.23162 | FANCG |
| AW024285 | 0.013622 | 0.024606 | 0.007049 | 0.311562 | FLJ12443 |
| W27666 | 0.013622 | 0.024606 | 0.009544 | −0.25685 | FLJ14393 |
| AA908993 | 0.013622 | 0.024606 | 0.015356 | −0.12684 | FLJ14393 |
| U90917 | 0.013622 | 0.024606 | 0.016602 | −0.23386 | FOXM1 |
| AF017445 | 0.013622 | 0.024606 | 0.025525 | −0.33517 | FPGT |
| AJ238764 | 0.013622 | 0.024606 | 0.030667 | 0.197763 | GNE |
| J04501 | 0.013622 | 0.024606 | 0.007821 | −0.23523 | GYS1 |
| X56841 | 0.013622 | 0.024606 | 0.022605 | −0.23469 | HLA-E |
| M63438 | 0.013622 | 0.024606 | 0.005389 | −0.75873 | IGKC |
| S66213 | 0.013622 | 0.024606 | 0.011362 | −0.09802 | ITGA6 |
| AB007870 | 0.013622 | 0.024606 | 0.002855 | 0.657213 | KIAA0410 |
| N29665 | 0.013622 | 0.024606 | 0.008301 | −0.49779 | KIAA0618 |
| AB018353 | 0.013622 | 0.024606 | 0.033864 | −0.3542 | KIAA0810 |
| D10522 | 0.013622 | 0.024606 | 0.028464 | 0.22227 | MACS |
| AF004709 | 0.013622 | 0.024606 | 0.018118 | −0.09931 | MAPK13 |
| W28275 | 0.013622 | 0.024606 | 0.005871 | −0.27591 | MGC11061 |
| AF087020 | 0.013622 | 0.024606 | 0.032064 | −0.13544 | MPZL1 |
| U61981 | 0.013622 | 0.024606 | 0.012069 | −0.20932 | MSH3 |
| U90942 | 0.013622 | 0.024606 | 0.004002 | 0.179029 | MYO5A |
| D50370 | 0.013622 | 0.024606 | 0.008327 | −0.11492 | NAP1L3 |
| U91512 | 0.013622 | 0.024606 | 0.00493 | 0.549889 | NINJ1 |
| AF069987 | 0.013622 | 0.024606 | 0.007336 | −0.16953 | NIT1 |
| U37689 | 0.013622 | 0.024606 | 0.007097 | −0.17369 | POLR2H |
| L19067 | 0.013622 | 0.024606 | 0.006406 | 0.148517 | RELA |
| X13482 | 0.013622 | 0.024606 | 0.016873 | 0.241998 | SNRPA1 |
| D16827 | 0.013622 | 0.024606 | 0.004314 | −0.16954 | SSTR5 |
| AB011420 | 0.013622 | 0.024606 | 0.030791 | 0.171669 | STK17A |
| L39060 | 0.013622 | 0.024606 | 0.026797 | −0.24028 | TAF1A |
| AB011169 | 0.013622 | 0.024606 | 0.004017 | −0.24355 | TEB4 |
| U69108 | 0.013622 | 0.024606 | 0.024472 | −0.17595 | TRAF5 |
| AB011004 | 0.013622 | 0.024606 | 0.00265 | 1.03158 | UAP1 |
| AB014584 | 0.013622 | 0.024606 | 0.028525 | −0.1337 | UBE4B |
| HG3914-HT4184 | 0.013622 | 0.024606 | 0.025854 | −0.12454 | |
| Z32860 | 0.013622 | 0.024606 | 0.002037 | −0.11041 | |
| U25849 | 0.013622 | 0.024606 | 0.00632 | −0.43498 | |
| AF052100 | 0.013622 | 0.024606 | 0.006718 | −0.2297 | |
| X59268 | 0.013622 | 0.024606 | 0.003586 | 0.479423 | GTF2B |
| AF007142 | 0.013622 | 0.024606 | 0.01864 | −0.34584 | |
| AI312646 | 0.013622 | 0.024606 | 0.027596 | −0.14991 | |
| AL022318 | 0.047678 | 0.028192 | 0.049355 | −0.11704 | APOBEC1L |
| M30704 | 0.047678 | 0.028192 | 0.00926 | 0.279668 | AREG |
| AF001307 | 0.047678 | 0.028192 | 0.018109 | −0.12594 | ARNT |
| AB020680 | 0.047678 | 0.028192 | 0.007009 | 0.227256 | BAG5 |
| AF018631 | 0.047678 | 0.028192 | 0.008344 | −0.13689 | BTD |
| D64110 | 0.047678 | 0.028192 | 0.022809 | 0.398412 | BTG3 |
| Z11697 | 0.047678 | 0.028192 | 0.024131 | 0.750492 | CD83 |
| M31516 | 0.047678 | 0.028192 | 0.021562 | 0.517068 | DAF |
| AF000982 | 0.047678 | 0.028192 | 0.025357 | 0.29808 | DDX3 |
| L77566 | 0.047678 | 0.028192 | 0.01243 | 0.178957 | DGSI |
| AL096725 | 0.047678 | 0.028192 | 0.007381 | 0.436688 | DKFZP434B103 |
| AL080201 | 0.047678 | 0.028192 | 0.044829 | −0.11576 | DKFZP434F162 |
| AL050286 | 0.047678 | 0.028192 | 0.004267 | −0.22397 | DKFZP586A011 |
| Y13350 | 0.047678 | 0.028192 | 0.015562 | 0.137002 | DNAJA2 |
| AJ223333 | 0.047678 | 0.028192 | 0.013836 | −0.17437 | DNMT2 |
| L34075 | 0.047678 | 0.028192 | 0.013848 | −0.25236 | FRAP1 |
| D31766 | 0.047678 | 0.028192 | 0.029287 | −0.09623 | GNPI |
| Z80776 | 0.047678 | 0.028192 | 0.002034 | 0.143491 | H2AFG |
| K03183 | 0.047678 | 0.028192 | 0.040298 | 0.163306 | HUMCGBBA3 |
| X57025 | 0.047678 | 0.028192 | 0.009135 | 0.437394 | IGF1 |
| X56681 | 0.047678 | 0.028192 | 0.012885 | 0.423181 | JUND |
| AB007916 | 0.047678 | 0.028192 | 0.00772 | −0.45744 | KIAA0447 |
| AI672098 | 0.047678 | 0.028192 | 0.014331 | 0.160649 | KIAA0934 |
| AB029020 | 0.047678 | 0.028192 | 0.035285 | −0.3101 | KIAA1097 |
| W27233 | 0.047678 | 0.028192 | 0.019918 | −0.24802 | KIDINS220 |
| AL049341 | 0.047678 | 0.028192 | 0.001943 | −0.3086 | LOC57209 |
| AL049422 | 0.047678 | 0.028192 | 0.028823 | 0.264129 | LOC84549 |
| AF010193 | 0.047678 | 0.028192 | 0.003729 | 0.927225 | MADH7 |
| AF007134 | 0.047678 | 0.028192 | 0.009151 | −0.1209 | MAPK8IP1 |
| L04731 | 0.047678 | 0.028192 | 0.025599 | −0.07236 | MLL |
| AB014547 | 0.047678 | 0.028192 | 0.023787 | −0.15831 | MTMR4 |
| U91616 | 0.047678 | 0.028192 | 0.018681 | 0.377931 | NFKBIE |
| X75918 | 0.047678 | 0.028192 | 0.019713 | 1.21948 | NR4A2 |
| AL049842 | 0.047678 | 0.028192 | 0.022698 | 0.201258 | NUFIP1 |
| U57843 | 0.047678 | 0.028192 | 0.011796 | −0.13969 | PIK3CD |
| S76965 | 0.047678 | 0.028192 | 0.012413 | 0.426208 | PKIA |
| AL023553 | 0.047678 | 0.028192 | 0.00321 | −0.15608 | PMM1 |
| M93425 | 0.047678 | 0.028192 | 0.019899 | −0.36854 | PTPN12 |
| AF044968 | 0.047678 | 0.028192 | 0.006887 | 0.121898 | PVRL2 |
| M28211 | 0.047678 | 0.028192 | 0.050065 | −0.08518 | RAB4 |
| AF083255 | 0.047678 | 0.028192 | 0.021248 | −0.27368 | RNAHP |
| U04897 | 0.047678 | 0.028192 | 0.013893 | 0.278167 | RORA |
| AL031228 | 0.047678 | 0.028192 | 0.020491 | −0.22382 | SACM2L |
| Y08262 | 0.047678 | 0.028192 | 0.008184 | −0.34195 | SCA2 |
| AF000652 | 0.047678 | 0.028192 | 0.001533 | 0.415218 | SDCBP |
| D31891 | 0.047678 | 0.028192 | 0.00536 | −0.18144 | SETDB1 |
| X66079 | 0.047678 | 0.028192 | 0.008707 | 0.129642 | SPIB |
| Z96932 | 0.047678 | 0.028192 | 0.013622 | −0.14003 | SSNA1 |
| D43642 | 0.047678 | 0.028192 | 0.015302 | −0.26409 | TCFL1 |
| D50919 | 0.047678 | 0.028192 | 0.016207 | −0.23972 | TRIM14 |
| X01703 | 0.047678 | 0.028192 | 0.004339 | 0.386096 | TUBA3 |
| AF022375 | 0.047678 | 0.028192 | 0.015198 | 0.503607 | VEGF |
| AF062346 | 0.047678 | 0.028192 | 0.014763 | 0.455053 | ZNF216 |
| J04755 | 0.047678 | 0.028192 | 0.014444 | 0.302274 | |
| AA524802 | 0.047678 | 0.028192 | 0.036226 | −0.24775 | |
| AL096749 | 0.047678 | 0.028192 | 0.017041 | 0.106309 | |
| M21259 | 0.047678 | 0.028192 | 0.025927 | 0.18378 | |
| X61587 | 0.047678 | 0.037364 | 0.024028 | 0.222788 | ARHG |
| J04027 | 0.047678 | 0.037364 | 0.019918 | 0.336927 | ATP2B1 |
| W28091 | 0.047678 | 0.037364 | 0.016936 | −0.1567 | BBS4 |
| U03106 | 0.047678 | 0.037364 | 0.004064 | 0.915096 | CDKN1A |
| AL049924 | 0.047678 | 0.037364 | 0.001517 | −0.23208 | DKFZP547G1110 |
| L19161 | 0.047678 | 0.037364 | 0.006578 | −0.49859 | EIF2S3 |
| AF052123 | 0.136189 | 0.037364 | 0.013524 | −0.24445 | FLJ10814 |
| AA522530 | 0.047678 | 0.037364 | 0.038021 | 0.413536 | FLJ20500 |
| AJ011001 | 0.047678 | 0.037364 | 0.016436 | −0.63045 | GPR56 |
| U50079 | 0.047678 | 0.037364 | 0.013178 | −0.37546 | HDAC1 |
| AI796944 | 0.047678 | 0.037364 | 0.011597 | 0.216392 | HIS1 |
| S82986 | 0.047678 | 0.037364 | 0.006441 | −0.20652 | HOXC6 |
| AB011173 | 0.047678 | 0.037364 | 0.01376 | −0.26283 | KIAA0601 |
| AB023160 | 0.047678 | 0.037364 | 0.029467 | −0.23276 | KIAA0943 |
| AJ001685 | 0.047678 | 0.037364 | 0.015208 | −0.48906 | KLRC3 |
| AJ000673 | 0.047678 | 0.037364 | 0.009021 | −0.38103 | KLRD1 |
| AB002450 | 0.047678 | 0.037364 | 0.003391 | −0.37426 | LOC51014 |
| U68385 | 0.047678 | 0.037364 | 0.007651 | −0.16327 | MEIS3 |
| AI688516 | 0.047678 | 0.037364 | 0.017859 | −0.15146 | NDUFA2 |
| W28770 | 0.047678 | 0.037364 | 0.005269 | −0.16121 | NP25 |
| L41827 | 0.047678 | 0.037364 | 0.011308 | 0.139896 | NRG1 |
| X84373 | 0.047678 | 0.037364 | 0.00725 | 0.77533 | NRIP1 |
| M25897 | 0.047678 | 0.037364 | 0.025747 | −0.41462 | PF4 |
| U50062 | 0.047678 | 0.037364 | 0.018154 | 0.19401 | RIPK1 |
| AJ011785 | 0.047678 | 0.037364 | 0.017907 | −0.07616 | SIX6 |
| X70683 | 0.047678 | 0.037364 | 0.0155 | −0.10219 | SOX4 |
| AL035699 | 0.047678 | 0.037364 | 0.006561 | −0.15185 | TBPL1 |
| D15050 | 0.047678 | 0.037364 | 0.016133 | 0.990791 | TCF8 |
| AF017146 | 0.047678 | 0.037364 | 0.002975 | −0.20652 | TOP3B |
| U54996 | 0.047678 | 0.037364 | 0.00691 | −0.17359 | ZW10 |
| HG4234-HT4504 | 0.047678 | 0.037364 | 0.003258 | −0.13985 | |
| X04500 | 0.047678 | 0.058634 | 0.02228 | 0.857952 | IL1B |

TABLE IV
Differential Gene Expression in MOG-reactive T-cells-MS vs.
Healthy

| | | | | Fold | Pvalue |
|---|---|---|---|---|---|
| Identifier | Symbol | Name | Function | Change | t-test |
| Up | | | | | |
| regulated | | | | | |
| M35878 | IGFBP3 | insulin-like growth | modulate IGF activity | 5.8 | 0.03 |
| | | factor binding protein 3 | | | |
| AB002318 | KIAA0320 | KIAA0320 protein | | 2.4 | 0.05 |
| AF024710 | VEGF | vascular endothelial | endothelial cell | 2.3 | 0.02 |
| | | growth factor | proliferation | | |
| AA628946 | KHSRP | KH-type splicing | mRNA processing | 2.2 | 0.01 |
| | | regulatory protein | | | |
| L42374 | PPP2R5B | protein phosphatase 2, | protein phosphatase | 2.1 | 0.05 |
| | | regulatory subunit B | | | |
| U54644 | TUB | tubby (mouse) | may be a | 1.8 | 0.01 |
| | | homolog | transcription factor | | |
| AB023167 | KIAA0950 | lifeguard | Apoptosis | 1.8 | 0.006 |
| X62654 | CD63 | CD63 antigen | growth regulation | 1.8 | 0.03 |
| | | (melanoma 1 antigen) | | | |
| H98552 | | cDNA | | 1.8 | 0.01 |
| | | DKFZp586I0523 | | | |
| AL050395 | MOF | member of MYST | histone acetyl | 1.7 | 0.03 |
| | | acetyl transferases | transferases | | |
| L27213 | SLC4A3 | solute carrier family 4, | inorganic anion | 1.7 | 0.01 |
| | | anion exchange 3 | exchanger | | |
| AF014837 | M6A | putative | Transcription factor | 1.6 | 0.05 |
| | | methyltransferase | | | |
| AB014537 | KIAA0637 | KIAA0637 gene | Apoptosis | 1.5 | 0.003 |
| | | product | | | |
| D13969 | ZNF144 | zinc finger protein 144 | DNA-Binding protein | 1.5 | 0.04 |
| | | (Mel-18) | | | |
| AJ012590 | H6PD | hexose-6-phosphate | Oxidoreductase | 1.5 | 0.04 |
| | | dehydrogenase | | | |
| M13995 | BCL2 | B-cell CLL/lymphoma 2 | Apoptosis | 1.5 | 0.03 |
| AI760801 | | chromosome 19, | | 1.5 | 0.009 |
| | | cosmid R31180 | | | |
| AI660963 | MAP3K12 | mitogen-activated | Transferase | 1.5 | 0.02 |
| | | protein 3 kinase 12 | cytoplasmic | | |
| Down | | | | | |
| regulated | | | | | |
| D45248 | PSME2 | proteasome activator | Protein degradation | −1.5 | 0.04 |
| | | subunit 2 (PA28 beta) | | | |
| W28612 | | ESTs | | −1.5 | 0.02 |
| Z46389 | VASP | vasodilator-stimulated | Signal transduction | −1.6 | 0.02 |
| | | phosphoprotein | | | |
| AA152202 | FLJ14639 | hypothetical protein | | −1.6 | 0.02 |
| | | FLJ14639 | | | |
| AF080561 | RBM14 | RNA binding motif | RNA binding protein | −1.7 | 0.03 |
| | | protein 14 | | | |
| D50922 | KIAA0132 | Kelch-like ECH- | ECH-associated | −1.7 | 0.03 |
| | | associated protein 1 | protein 1 | | |
| AF025441 | OIP5 | Opa-interacting protein 5 | | −1.8 | 0.04 |
| AF080227 | EED | embryonic ectoderm | transcriptional | −1.8 | 0.04 |
| | | development | repressor | | |
| D87957 | RQCD1 | required for cell | sex differentiation | −1.9 | 0.03 |
| | | differentiation | | | |
| X61498 | NFKB2 | nuclear factor of kappa | expression of | −1.9 | 0.05 |
| | | light polypeptide | inflammatory genes | | |
| | | Bcells | | | |
| X52425 | IL4R | interleukin 4 receptor | receptor signalling | −2 | 0.04 |
| | | | protein | | |
| L08069 | DNAJA1 | DnaJ (Hsp40) | protein folding and | −2 | 0.04 |
| | | homolog, subfamily A, | transport | | |
| | | member 1 | | | |
| AF071504 | STX11 | syntaxin 11 | protein transport | −2.1 | 0.03 |
| M11717 | HSPA1A | heat shock 70 kD | heat shock response | −2.2 | 0.03 |
| | | protein 1A | | | |
| M59830 | HSPA1B | heat shock 70 kD | heat shock response | −2.2 | 0.03 |
| | | protein 1B | | | |
| M16441 | TNF | Human tumor necrosis | Inflammatory | −2.3 | 0.05 |
| | | factor | response | | |
| D89077 | SLA | Src-like-adapter | | −2.4 | 0.05 |
| U77949 | CDC6 | cell division cycle 6, S. cerevisiae | DNA replication | −2.5 | 0.02 |
| | | homolog | checkpoint | | |
| D38549 | KIAA0068 | KIAA0068 protein | | −2.5 | 0.01 |
| L23959 | TFDP1 | transcription factor Dp-1 | cycle progression G1 | −2.5 | 0.01 |
| | | | to S-phase | | |
| L78833 | BRCA1 | Breast cancer | | −2.7 | 0.04 |
| | | susceptibility gene | | | |
| M63193 | ECGF1 | endothelial cell growth | stimulates | −2.8 | 0.01 |
| | | factor 1 | angiogenesis | | |
| AF035625 | STK11 | serine/threonine kinase | Peutz-Jeghers | −2.9 | 0.04 |
| | | 11 | syndrome | | |
| J04130 | SCYA4 | small inducible | Cell-to-cell signalling | −2.9 | 0.05 |
| | | cytokine A4 | | | |
| X93086 | BLVRA | biliverdin reductase A | biliverdin reductase | −4 | 0.03 |

TABLE V
Differential Gene Expression in Probable MS vs. Healthy

| | | | t-Test | Log Fold | |
|---|---|---|---|---|---|
| Identification | TNOM PValue | Info PValue | PValue | Change | Gene Symbol |
| NM_018049.1 | 0.000233 | 0.000233 | 2.46E−05 | 0.438337 | FLJ10297 |
| NM_005886.1 | 0.000233 | 0.000233 | 0.000553 | 0.35972 | KATNB1 |
| NM_000161.1 | 0.000233 | 0.000233 | 0.000297 | −0.48848 | GCH1 |
| NM_001539.1 | 0.000233 | 0.000233 | 0.000144 | −0.58017 | DNAJA1 |
| AF349571.1 | 0.004202 | 0.004202 | 0.000274 | 1.78925 | HBA1 |
| M25079.1 | 0.004202 | 0.004202 | 0.000247 | 1.59503 | HBB |
| V00489 | 0.004202 | 0.004202 | 0.000268 | 1.54947 | |
| BC005931.1 | 0.004202 | 0.004202 | 0.000296 | 1.48707 | HBA2 |
| T50399 | 0.004202 | 0.004202 | 0.000275 | 1.43533 | HBA2 |
| NM_024567.1 | 0.004202 | 0.004202 | 0.002206 | 1.42146 | FLJ21616 |
| AF105974.1 | 0.004202 | 0.004202 | 0.001086 | 1.3896 | HBA1 |
| NM_000558.2 | 0.004202 | 0.004202 | 0.000707 | 1.3348 | HBA1 |
| AI133353 | 0.004202 | 0.004202 | 0.000897 | 1.29746 | HBG2 |
| AF059180 | 0.004202 | 0.004202 | 0.000309 | 1.29355 | |
| AF349114.1 | 0.004202 | 0.004202 | 0.000163 | 1.27511 | HBB |
| BE547674 | 0.004202 | 0.004202 | 0.002947 | 0.636619 | |
| NM_012452.1 | 0.004202 | 0.004202 | 0.000541 | 0.570818 | TNFRSF13B |
| AA314406 | 0.004202 | 0.001401 | 0.002013 | 0.520631 | TRAP95 |
| NM_015909.1 | 0.004202 | 0.001401 | 0.000398 | 0.501733 | NAG |
| NM_006868.1 | 0.004202 | 0.004202 | 0.002711 | 0.49862 | RAB31 |
| BC000305.1 | 0.004202 | 0.004202 | 0.006921 | 0.475733 | CASP6 |
| L77566 | 0.004202 | 0.001401 | 0.00033 | 0.446293 | DGSI |
| BF971416 | 0.004202 | 0.004202 | 0.002405 | 0.410742 | DKFZP586N0721 |
| BE879367 | 0.004202 | 0.004202 | 0.00056 | 0.382484 | AKAP2 |
| NM_001640.2 | 0.004202 | 0.001401 | 0.004091 | 0.36631 | APEH |
| BC001808.1 | 0.004202 | 0.004202 | 0.012997 | 0.347043 | NM23-H6 |
| AL049539 | 0.004202 | 0.004202 | 0.010738 | 0.2822 | KIAA0255 |
| BC000580.1 | 0.004202 | 0.001401 | 0.014166 | 0.270658 | PH-4 |
| NM_012151.2 | 0.004202 | 0.001401 | 0.000685 | 0.267664 | F8A |
| BC004423.1 | 0.004202 | 0.004202 | 0.012194 | 0.249144 | TNRC5 |
| NM_004890.1 | 0.004202 | 0.001401 | 0.010332 | 0.134439 | SPAG7 |
| AB029040 | 0.004202 | 0.004202 | 0.010819 | −0.14172 | KIAA1117 |
| NM_025160.1 | 0.004202 | 0.001401 | 0.004986 | −0.21141 | FLJ21016 |
| AW162015 | 0.004202 | 0.004202 | 0.301224 | −0.24766 | ZNF143 |
| NM_005574.2 | 0.004202 | 0.004202 | 0.107595 | −0.24939 | LMO2 |
| NM_014670.1 | 0.004202 | 0.004202 | 0.025534 | −0.27225 | BZW1 |
| AL117643.1 | 0.004202 | 0.004202 | 0.046495 | −0.27766 | |
| AA628948 | 0.004202 | 0.001401 | 0.000319 | −0.28951 | ADSS |
| AF251062.1 | 0.004202 | 0.001401 | 0.000435 | −0.30924 | LOC84549 |
| AL564683 | 0.004202 | 0.004202 | 0.018258 | −0.44633 | CEBPB |
| NM_014999.1 | 0.004202 | 0.004202 | 0.001021 | −0.47304 | RAB21 |
| NM_017723.1 | 0.004202 | 0.004202 | 0.02759 | −0.49548 | FLJ20245 |
| NM_003264.1 | 0.004202 | 0.004202 | 0.002592 | −0.49551 | TLR2 |
| AF062347.1 | 0.004202 | 0.004202 | 0.00529 | −0.51432 | ZNF216 |
| NM_004556.1 | 0.004202 | 0.004202 | 0.001134 | −0.53489 | NFKBIE |
| U92014.1 | 0.004202 | 0.004202 | 0.003064 | −0.59511 | |
| NM_014778.1 | 0.004202 | 0.004202 | 0.001106 | −0.65156 | KIAA0410 |
| NM_015384.1 | 0.004202 | 0.004202 | 0.002943 | −0.68193 | IDN3 |
| AK022513.1 | 0.004202 | 0.004202 | 0.001326 | −0.68416 | DUSP10 |
| NM_003246.1 | 0.004202 | 0.001401 | 0.000162 | −1.44745 | THBS1 |
| AI812030 | 0.004202 | 0.001401 | 7.42E−05 | −1.51098 | THBS1 |
| NM_000559.1 | 0.035714 | 0.035247 | 0.006202 | 1.93991 | HBG1 |
| NM_000184.1 | 0.035714 | 0.04225 | 0.009164 | 1.67513 | HBG2 |
| NM_005564.1 | 0.035714 | 0.04225 | 0.010814 | 1.03322 | LCN2 |
| AF274863.1 | 0.035714 | 0.00747 | 0.007719 | 0.952868 | SEC31B-1 |
| NM_002288.2 | 0.035714 | 0.015406 | 0.010689 | 0.834313 | LAIR2 |
| M87789.1 | 0.035714 | 0.04225 | 0.058518 | 0.79032 | IGHG3 |
| NM_005764.1 | 0.035714 | 0.04225 | 0.053679 | 0.789147 | DD96 |
| AK000168.1 | 0.035714 | 0.04225 | 0.038176 | 0.763766 | KIAA1919 |
| NM_020037.1 | 0.035714 | 0.035247 | 0.061379 | 0.746143 | ABCC3 |
| AF103529.1 | 0.035714 | 0.035247 | 0.015284 | 0.736268 | |
| AV698647 | 0.035714 | 0.035247 | 0.017411 | 0.616137 | IGLJ3 |
| AI357539 | 0.035714 | 0.04225 | 0.00558 | 0.601843 | MGC4126 |
| NM_015935.1 | 0.035714 | 0.035247 | 0.021683 | 0.593818 | CGI-01 |
| D38535 | 0.035714 | 0.04225 | 0.016025 | 0.571168 | ITIH4 |
| AA723370 | 0.035714 | 0.04225 | 0.039917 | 0.570325 | LOC51011 |
| AF227968.1 | 0.035714 | 0.00747 | 0.005308 | 0.566516 | SH2B |
| X12530.1 | 0.035714 | 0.04225 | 0.056335 | 0.559172 | MS4A1 |
| AI348935 | 0.035714 | 0.035247 | 0.014129 | 0.546773 | CALR |
| NM_003422.1 | 0.035714 | 0.04225 | 0.006737 | 0.536815 | ZNF42 |
| NM_015559.1 | 0.035714 | 0.04225 | 0.028795 | 0.536316 | SETBP1 |
| NM_013378.1 | 0.035714 | 0.035247 | 0.005191 | 0.532564 | VPREB3 |
| NM_004912.1 | 0.035714 | 0.015406 | 0.003037 | 0.52082 | CCM1 |
| NM_006230.1 | 0.035714 | 0.015406 | 0.007471 | 0.517979 | POLD2 |
| NM_006235.1 | 0.035714 | 0.035247 | 0.021726 | 0.515263 | POU2AF1 |
| AL037557 | 0.035714 | 0.00747 | 0.002786 | 0.511724 | POLR2I |
| NM_014703.1 | 0.035714 | 0.00747 | 0.006709 | 0.4938 | KIAA0800 |
| NM_015670.1 | 0.035714 | 0.035247 | 0.045708 | 0.464156 | SENP3 |
| AA643304 | 0.035714 | 0.015406 | 0.004712 | 0.459694 | |
| AI948503 | 0.035714 | 0.04225 | 0.017964 | 0.45625 | ABCC4 |
| BC002807.1 | 0.035714 | 0.04225 | 0.100759 | 0.455683 | MS4A1 |
| AF123539.1 | 0.035714 | 0.035247 | 0.028286 | 0.454603 | HTCD37 |
| AA149644 | 0.035714 | 0.00747 | 0.02413 | 0.450082 | JAM3 |
| BC000585.1 | 0.035714 | 0.04225 | 0.033113 | 0.44368 | SLC21A11 |
| AB044806.1 | 0.035714 | 0.04225 | 0.007766 | 0.433985 | KCNH2 |
| U37025 | 0.035714 | 0.035247 | 0.046971 | 0.427213 | SULT1A1 |
| NM_020166.2 | 0.035714 | 0.035247 | 0.006318 | 0.423798 | MCCC1 |
| NM_002876.1 | 0.035714 | 0.035247 | 0.026644 | 0.414867 | RAD51C |
| NM_002387.1 | 0.035714 | 0.035247 | 0.01092 | 0.409481 | MCC |
| NM_005816.1 | 0.035714 | 0.04225 | 0.103051 | 0.407348 | TACTILE |
| H95263 | 0.035714 | 0.035247 | 0.069113 | 0.406766 | |
| NM_003146.1 | 0.035714 | 0.035247 | 0.003146 | 0.405309 | SSRP1 |
| NM_003550.1 | 0.035714 | 0.035247 | 0.054105 | 0.403851 | MAD1L1 |
| AK022494.1 | 0.035714 | 0.00747 | 0.001189 | 0.397073 | RAB3GAP |
| NM_006400.2 | 0.035714 | 0.035247 | 0.021072 | 0.396297 | DCTN2 |
| NM_006012.1 | 0.035714 | 0.035247 | 0.015241 | 0.394779 | CLPP |
| NM_014921.1 | 0.035714 | 0.035247 | 0.014463 | 0.394308 | LEC2 |
| NM_025056.1 | 0.035714 | 0.00747 | 0.01912 | 0.393739 | FLJ23185 |
| NM_003573.1 | 0.035714 | 0.04225 | 0.026053 | 0.393337 | LTBP4 |
| NM_000132.2 | 0.035714 | 0.035247 | 0.005292 | 0.392442 | F8 |
| AF031824.1 | 0.035714 | 0.035247 | 0.190243 | 0.389129 | CST7 |
| NM_001841.1 | 0.035714 | 0.035247 | 0.07115 | 0.387067 | CNR2 |
| NM_018391.1 | 0.035714 | 0.035247 | 0.138584 | 0.386982 | FLJ23277 |
| U79248.1 | 0.035714 | 0.00747 | 0.007475 | 0.386419 | |
| NM_024332.1 | 0.035714 | 0.04225 | 0.039154 | 0.386198 | C6.1A |
| BF510692 | 0.035714 | 0.04225 | 0.046782 | 0.385324 | PAX5 |
| AA243774 | 0.035714 | 0.035247 | 0.050456 | 0.381631 | MMP24 |
| AL121964 | 0.035714 | 0.035247 | 0.107681 | 0.373759 | MAP3K7 |
| L25275.1 | 0.035714 | 0.035247 | 0.020684 | 0.372971 | SULT1A3 |
| AB018289.1 | 0.035714 | 0.035247 | 0.002286 | 0.37263 | KIAA0746 |
| NM_000294.1 | 0.035714 | 0.035247 | 0.011432 | 0.367693 | PHKG2 |
| BC001906.1 | 0.035714 | 0.035247 | 0.107947 | 0.366899 | MTX1 |
| NM_000651.1 | 0.035714 | 0.04225 | 0.047953 | 0.365471 | CR1 |
| NM_001667.1 | 0.035714 | 0.00747 | 0.009661 | 0.362105 | ARL2 |
| AI133727 | 0.035714 | 0.00747 | 0.018354 | 0.358884 | ZAP |
| BC002873.1 | 0.035714 | 0.035247 | 0.048711 | 0.358052 | DKFZP564J0123 |
| NM_004178.2 | 0.035714 | 0.035247 | 0.007313 | 0.356459 | TARBP2 |
| BG532929 | 0.035714 | 0.035247 | 0.037215 | 0.356254 | SSB |
| NM_018094.1 | 0.035714 | 0.035247 | 0.014302 | 0.351314 | GSPT2 |
| AC004531 | 0.035714 | 0.00747 | 0.01714 | 0.350445 | DDX28 |
| NM_001981.1 | 0.035714 | 0.035247 | 0.010776 | 0.347051 | EPS15 |
| AB020689.1 | 0.035714 | 0.035247 | 0.024594 | 0.346253 | KIAA0882 |
| NM_001055.1 | 0.035714 | 0.035247 | 0.056416 | 0.344937 | SULT1A1 |
| NM_022067.1 | 0.035714 | 0.035247 | 0.001332 | 0.337713 | FLJ12707 |
| NM_000195.1 | 0.035714 | 0.00747 | 0.014156 | 0.3312 | HPS1 |
| NM_022914.1 | 0.035714 | 0.00747 | 0.167735 | 0.331082 | 24432 |
| NM_003627.1 | 0.035714 | 0.035247 | 0.062759 | 0.330371 | POV1 |
| NM_022060.1 | 0.035714 | 0.035247 | 0.010717 | 0.328122 | FLJ12816 |
| BF446180 | 0.035714 | 0.035247 | 0.02537 | 0.326347 | PDCD2 |
| U28169.1 | 0.035714 | 0.035247 | 0.0891 | 0.326218 | SULT1A2 |
| AF316873.1 | 0.035714 | 0.035247 | 0.022727 | 0.3257 | PINK1 |
| NM_017615.1 | 0.035714 | 0.035247 | 0.087717 | 0.325056 | FLJ20003 |
| NM_015853.1 | 0.035714 | 0.035247 | 0.021537 | 0.321089 | LOC51035 |
| NM_018449.1 | 0.035714 | 0.035247 | 0.01461 | 0.318475 | UBAP2 |
| NM_007056.1 | 0.035714 | 0.035247 | 0.013827 | 0.318086 | SWAP2 |
| AV702994 | 0.035714 | 0.035247 | 0.010766 | 0.316138 | LOC51668 |
| AK021884.1 | 0.035714 | 0.04225 | 0.016862 | 0.315879 | NPEPPS |
| U64898.1 | 0.035714 | 0.035247 | 0.012705 | 0.309446 | NRD1 |
| AI431902 | 0.035714 | 0.035247 | 0.026163 | 0.307321 | FLJ13491 |
| NM_003689.1 | 0.035714 | 0.04225 | 0.03366 | 0.306281 | AKR7A2 |
| BE791629 | 0.035714 | 0.00747 | 0.039522 | 0.304821 | CGTHBA |
| NM_016194.1 | 0.035714 | 0.035247 | 0.060249 | 0.303474 | GNB5 |
| NM_014965.1 | 0.035714 | 0.035247 | 0.013547 | 0.298042 | KIAA1042 |
| NM_003363.1 | 0.035714 | 0.035247 | 0.018393 | 0.295239 | USP4 |
| U88964 | 0.035714 | 0.035247 | 0.019349 | 0.294427 | ISG20 |
| BC001782.1 | 0.035714 | 0.035247 | 0.059026 | 0.293156 | GAS2L1 |
| BC004361.1 | 0.035714 | 0.04225 | 0.084538 | 0.292509 | PSCD2 |
| NM_017840.1 | 0.035714 | 0.035247 | 0.003929 | 0.290472 | MRPL16 |
| NM_006321.1 | 0.035714 | 0.015406 | 0.006688 | 0.288723 | ARIH2 |
| AI341234 | 0.035714 | 0.035247 | 0.007356 | 0.284699 | CORO1B |
| N20923 | 0.035714 | 0.035247 | 0.020271 | 0.280552 | FYN |
| L42531.1 | 0.035714 | 0.035247 | 0.008554 | 0.280023 | |
| AK000818.1 | 0.035714 | 0.035247 | 0.02226 | 0.277695 | FLJ20811 |
| NM_000633.1 | 0.035714 | 0.035247 | 0.044639 | 0.276897 | BCL2 |
| BE551347 | 0.035714 | 0.035247 | 0.209003 | 0.276406 | FLJ13052 |
| AK000161.1 | 0.035714 | 0.04225 | 0.016752 | 0.276103 | FLJ20154 |
| AI798908 | 0.035714 | 0.04225 | 0.015969 | 0.274921 | KIAA0226 |
| NM_005111.1 | 0.035714 | 0.035247 | 0.01405 | 0.273732 | CRYZL1 |
| NM_024551.1 | 0.035714 | 0.035247 | 0.00372 | 0.272684 | FLJ21432 |
| BC006214.1 | 0.035714 | 0.00747 | 0.006244 | 0.268704 | IRO039700 |
| AI123527 | 0.035714 | 0.04225 | 0.105392 | 0.268349 | KIAA0092 |
| NM_004379.1 | 0.035714 | 0.035247 | 0.047229 | 0.267796 | CREB1 |
| AA643304 | 0.035714 | 0.035247 | 0.039678 | 0.258201 | |
| NM_013417.1 | 0.035714 | 0.035247 | 0.047087 | 0.257738 | IARS |
| AK025432.1 | 0.035714 | 0.035247 | 0.051871 | 0.257456 | KIAA0564 |
| AB028960 | 0.035714 | 0.04225 | 0.040942 | 0.254827 | KIAA1037 |
| NM_000048.1 | 0.035714 | 0.04225 | 0.038931 | 0.254447 | ASL |
| NM_002808.1 | 0.035714 | 0.035247 | 0.023966 | 0.250129 | PSMD2 |
| NM_001054.1 | 0.035714 | 0.035247 | 0.062598 | 0.248696 | SULT1A2 |
| NM_005428.2 | 0.035714 | 0.035247 | 0.007185 | 0.248439 | VAV1 |
| NM_022758.1 | 0.035714 | 0.04225 | 0.011489 | 0.246401 | FLJ22195 |
| AY009128.1 | 0.035714 | 0.035247 | 0.084938 | 0.246257 | NIFU |
| AB017004.1 | 0.035714 | 0.035247 | 0.079567 | 0.244954 | PMS2L8 |
| NM_000249.1 | 0.035714 | 0.04225 | 0.021274 | 0.243441 | MLH1 |
| U51007.1 | 0.035714 | 0.035247 | 0.042753 | 0.242223 | |
| BC002640.1 | 0.035714 | 0.035247 | 0.074751 | 0.240603 | |
| NM_016284.1 | 0.035714 | 0.00747 | 0.001929 | 0.240076 | KIAA1007 |
| NM_002414.1 | 0.035714 | 0.035247 | 0.063998 | 0.239013 | MIC2 |
| BC000212.1 | 0.035714 | 0.035247 | 0.021052 | 0.237577 | GTF3C2 |
| NM_004398.2 | 0.035714 | 0.035247 | 0.040656 | 0.235252 | DDX10 |
| NM_024713.1 | 0.035714 | 0.035247 | 0.048887 | 0.234927 | FLJ22557 |
| NM_002810.1 | 0.035714 | 0.035247 | 0.038558 | 0.234593 | PSMD4 |
| NM_030580.1 | 0.035714 | 0.035247 | 0.031263 | 0.233466 | MGC10520 |
| AB007896.1 | 0.035714 | 0.035247 | 0.211816 | 0.231563 | KIAA0436 |
| NM_003954.1 | 0.035714 | 0.04225 | 0.051916 | 0.230862 | MAP3K14 |
| NM_025207.1 | 0.035714 | 0.035247 | 0.020823 | 0.230015 | PP591 |
| NM_016323.1 | 0.035714 | 0.035247 | 0.051393 | 0.228764 | LOC51191 |
| NM_016069.1 | 0.035714 | 0.04225 | 0.132766 | 0.223618 | Magmas |
| NM_013349.1 | 0.035714 | 0.035247 | 0.028748 | 0.223073 | SPUF |
| NM_000884.1 | 0.035714 | 0.035247 | 0.064347 | 0.222421 | IMPDH2 |
| BG167570 | 0.035714 | 0.04225 | 0.108243 | 0.219652 | DKFZp762N1910 |
| NM_004551.1 | 0.035714 | 0.00747 | 0.06025 | 0.217427 | NDUFS3 |
| BG231932 | 0.035714 | 0.04225 | 0.079649 | 0.210447 | CLN2 |
| NM_017851.1 | 0.035714 | 0.035247 | 0.036308 | 0.209956 | FLJ20509 |
| NM_006519.1 | 0.035714 | 0.035247 | 0.032991 | 0.202387 | TCTEL1 |
| AF032900.1 | 0.035714 | 0.035247 | 0.174549 | 0.200739 | COQ7 |
| AL535380 | 0.035714 | 0.035247 | 0.265205 | 0.198073 | BTG1 |
| AW118862 | 0.035714 | 0.035247 | 0.02173 | 0.193753 | RREB1 |
| NM_000382.1 | 0.035714 | 0.035247 | 0.27948 | 0.193509 | ALDH3A2 |
| NM_024419.1 | 0.035714 | 0.035247 | 0.164883 | 0.190623 | PGS1 |
| NM_003904.1 | 0.035714 | 0.035247 | 0.245676 | 0.190422 | ZNF259 |
| AI928526 | 0.035714 | 0.00747 | 0.036861 | 0.185624 | JTV1 |
| NM_024581.1 | 0.035714 | 0.035247 | 0.230867 | 0.185323 | FLJ13942 |
| AF085357.1 | 0.035714 | 0.035247 | 0.110175 | 0.184965 | FLOT1 |
| NM_004475.1 | 0.035714 | 0.035247 | 0.072642 | 0.180483 | FLOT2 |
| AF334103.1 | 0.035714 | 0.00747 | 0.009664 | 0.17511 | GU2 |
| NM_017829.1 | 0.035714 | 0.035247 | 0.110207 | 0.174515 | CECR5 |
| NM_004214.3 | 0.035714 | 0.04225 | 0.016835 | 0.157902 | FIBP |
| NM_017704.1 | 0.035714 | 0.04225 | 0.16159 | 0.157672 | FLJ20189 |
| NM_003592.1 | 0.035714 | 0.035247 | 0.038652 | 0.146241 | CUL1 |
| AI537887 | 0.035714 | 0.035247 | 0.467375 | 0.139355 | EPB72 |
| NM_023935.1 | 0.035714 | 0.035247 | 0.049119 | 0.125305 | C20orf116 |
| BG398414 | 0.035714 | 0.035247 | 0.286856 | 0.123085 | RPA1 |
| NM_016243.1 | 0.035714 | 0.035247 | 0.279995 | 0.121894 | LOC51706 |
| NM_012199.1 | 0.035714 | 0.035247 | 0.093241 | 0.118547 | EIF2C1 |
| AK024029.1 | 0.035714 | 0.04225 | 0.450393 | 0.11646 | MAP-1 |
| NM_004848.1 | 0.035714 | 0.035247 | 0.486492 | 0.113516 | ICB-1 |
| AF144638.1 | 0.035714 | 0.035247 | 0.255571 | 0.10089 | SGPL1 |
| D86062.1 | 0.035714 | 0.035247 | 0.532398 | 0.084417 | C21orf33 |
| NM_000655.2 | 0.035714 | 0.035247 | 0.535745 | 0.081167 | SELL |
| NM_018643.1 | 0.035714 | 0.035247 | 0.870775 | 0.057399 | TREM1 |
| NM_018326.1 | 0.035714 | 0.035247 | 0.929375 | 0.035048 | HIMAP4 |
| NM_005371.2 | 0.035714 | 0.035247 | 0.876737 | 0.025127 | METTL1 |
| NM_007002.1 | 0.035714 | 0.035247 | 0.911541 | 0.010422 | ADRM1 |
| NM_004723.1 | 0.035714 | 0.035247 | 0.975685 | −0.00562 | ARHGEF2 |
| U31501 | 0.035714 | 0.035247 | 0.724549 | −0.0658 | FXR2 |
| NM_005338.3 | 0.035714 | 0.04225 | 0.126911 | −0.0661 | HIP1 |
| AB006589.1 | 0.035714 | 0.035247 | 0.00542 | −0.10655 | ESR2 |
| AA868754 | 0.035714 | 0.035247 | 0.304519 | −0.10746 | KIAA0650 |
| AU144792 | 0.035714 | 0.035247 | 0.008623 | −0.11362 | |
| AF320999.1 | 0.035714 | 0.035247 | 0.289096 | −0.11449 | RTN4 |
| NM_013229.1 | 0.035714 | 0.035247 | 0.529181 | −0.13735 | APAF1 |
| NM_018690.1 | 0.035714 | 0.04225 | 0.261146 | −0.14482 | APOB48R |
| D42055.1 | 0.035714 | 0.04225 | 0.007978 | −0.14841 | NEDD4 |
| BF968633 | 0.035714 | 0.035247 | 0.135003 | −0.14873 | RNF4 |
| AK026678.1 | 0.035714 | 0.035247 | 0.00833 | −0.15056 | STAG2 |
| NM_014671.1 | 0.035714 | 0.035247 | 0.392979 | −0.15386 | KIAA0010 |
| NM_030979.1 | 0.035714 | 0.035247 | 0.087494 | −0.15652 | PABPC3 |
| BG429214 | 0.035714 | 0.035247 | 0.273519 | −0.15766 | |
| NM_006892.1 | 0.035714 | 0.035247 | 0.001922 | −0.15828 | DNMT3B |
| NM_018975.1 | 0.035714 | 0.035247 | 0.042202 | −0.16723 | RAP1 |
| AL137335.1 | 0.035714 | 0.035247 | 0.306422 | −0.17292 | RANBP7 |
| NM_014016.1 | 0.035714 | 0.035247 | 0.398036 | −0.17365 | SACM1L |
| NM_012198.1 | 0.035714 | 0.035247 | 0.391555 | −0.17556 | GCA |
| NM_024586.1 | 0.035714 | 0.04225 | 0.011965 | −0.19298 | OSBPL9 |
| N64643 | 0.035714 | 0.035247 | 0.16498 | −0.19313 | KIAA0625 |
| NM_005951.1 | 0.035714 | 0.035247 | 0.156965 | −0.1942 | MT1H |
| NM_002264.1 | 0.035714 | 0.035247 | 0.138195 | −0.1949 | |
| AF182415.1 | 0.035714 | 0.04225 | 0.325959 | −0.19495 | RBM8A |
| BE674061 | 0.035714 | 0.035247 | 0.015036 | −0.20133 | PIN4 |
| NM_004973.2 | 0.035714 | 0.00747 | 0.103071 | −0.20162 | JMJ |
| U58852.1 | 0.035714 | 0.035247 | 0.510508 | −0.20606 | NPAT |
| NM_005565.2 | 0.035714 | 0.035247 | 0.037541 | −0.2105 | LCP2 |
| NM_004941.1 | 0.035714 | 0.035247 | 0.229189 | −0.21215 | DDX8 |
| U02297.1 | 0.035714 | 0.035247 | 0.252672 | −0.21782 | SELPLG |
| NM_002940.1 | 0.035714 | 0.035247 | 0.112373 | −0.22731 | ABCE1 |
| AL550657 | 0.035714 | 0.035247 | 0.069403 | −0.23303 | BSG |
| BG387770 | 0.035714 | 0.035247 | 0.032984 | −0.2362 | MGC32104 |
| AL050205.1 | 0.035714 | 0.04225 | 0.352078 | −0.23748 | LOC113251 |
| NM_016653.1 | 0.035714 | 0.035247 | 0.003387 | −0.23765 | ZAK |
| AA742237 | 0.035714 | 0.035247 | 0.120935 | −0.23853 | BAT2 |
| NM_021183.1 | 0.035714 | 0.035247 | 0.069121 | −0.24239 | LOC57826 |
| AB014527.1 | 0.035714 | 0.035247 | 0.005636 | −0.24315 | CLASP2 |
| AF091086.1 | 0.035714 | 0.035247 | 0.124853 | −0.24621 | CL640 |
| NM_006748.1 | 0.035714 | 0.04225 | 0.141473 | −0.24728 | SLA |
| NM_025238.1 | 0.035714 | 0.035247 | 0.046507 | −0.24841 | BTBD1 |
| NM_018638.2 | 0.035714 | 0.035247 | 0.074405 | −0.24942 | EKI1 |
| NM_002913.1 | 0.035714 | 0.04225 | 0.092197 | −0.24967 | |
| NM_002863.1 | 0.035714 | 0.035247 | 0.034567 | −0.25494 | PYGL |
| AF226044.1 | 0.035714 | 0.035247 | 0.023966 | −0.25679 | SNRK |
| NM_016217.1 | 0.035714 | 0.035247 | 0.01673 | −0.25733 | LOC51696 |
| AF084943.1 | 0.035714 | 0.035247 | 0.024841 | −0.26011 | MINPP1 |
| N22548 | 0.035714 | 0.04225 | 0.03686 | −0.26164 | ROCK1 |
| AF033850.1 | 0.035714 | 0.035247 | 0.110532 | −0.26338 | PLD2 |
| NM_014445.1 | 0.035714 | 0.00747 | 0.007058 | −0.26858 | SERP1 |
| NM_016196.1 | 0.035714 | 0.035247 | 0.014278 | −0.27109 | KIAA0682 |
| NM_012252.1 | 0.035714 | 0.035247 | 0.020625 | −0.27124 | TFEC |
| W72082 | 0.035714 | 0.035247 | 0.180599 | −0.27284 | C1QR1 |
| NM_016166.1 | 0.035714 | 0.035247 | 0.071782 | −0.27429 | PIAS1 |
| NM_022470.1 | 0.035714 | 0.035247 | 0.072884 | −0.27655 | WIG1 |
| NM_030797.1 | 0.035714 | 0.035247 | 0.039197 | −0.27728 | DKFZP566A1524 |
| NM_002199.2 | 0.035714 | 0.035247 | 0.250656 | −0.27789 | IRF2 |
| BC003360.1 | 0.035714 | 0.035247 | 0.02171 | −0.27851 | DDX18 |
| NM_004504.2 | 0.035714 | 0.035247 | 0.020834 | −0.27873 | HRB |
| NM_012072.2 | 0.035714 | 0.035247 | 0.118494 | −0.27892 | C1QR1 |
| NM_018230.1 | 0.035714 | 0.035247 | 0.071301 | −0.28019 | NUP133 |
| NM_002727.1 | 0.035714 | 0.04225 | 0.038559 | −0.28438 | PRG1 |
| BC005338.1 | 0.035714 | 0.035247 | 0.114062 | −0.28524 | CAPZA2 |
| U60521.1 | 0.035714 | 0.04225 | 0.066643 | −0.29174 | CASP9 |
| AW188198 | 0.035714 | 0.035247 | 0.005126 | −0.29178 | TNFAIP6 |
| BE908931 | 0.035714 | 0.035247 | 0.017009 | −0.29572 | |
| U64661 | 0.035714 | 0.04225 | 0.030982 | −0.29704 | |
| AL021395 | 0.035714 | 0.04225 | 0.02084 | −0.29857 | |
| NM_015176.1 | 0.035714 | 0.035247 | 0.059688 | −0.299 | KIAA0483 |
| NM_002857.1 | 0.035714 | 0.04225 | 0.04761 | −0.29944 | PXF |
| U70451.1 | 0.035714 | 0.035247 | 0.003458 | −0.30169 | MYD88 |
| NM_018042.1 | 0.035714 | 0.035247 | 0.020209 | −0.30409 | FLJ10260 |
| AL049265.1 | 0.035714 | 0.04225 | 0.136618 | −0.30472 | |
| NM_024081.1 | 0.035714 | 0.035247 | 0.020267 | −0.30513 | TMG4 |
| AI796169 | 0.035714 | 0.035247 | 0.023959 | −0.31104 | GATA3 |
| AA160522 | 0.035714 | 0.035247 | 0.056044 | −0.31114 | UBE3A |
| AL136621.1 | 0.035714 | 0.04225 | 0.06859 | −0.31538 | ZNF198 |
| NM_003051.1 | 0.035714 | 0.035247 | 0.028776 | −0.3171 | SLC16A1 |
| AW572909 | 0.035714 | 0.035247 | 0.027025 | −0.31713 | KIAA0874 |
| NM_017782.1 | 0.035714 | 0.04225 | 0.017104 | −0.32078 | FLJ20360 |
| AK001821.1 | 0.035714 | 0.04225 | 0.013182 | −0.32145 | MGC4170 |
| AW001847 | 0.035714 | 0.035247 | 0.204195 | −0.32259 | APLP2 |
| BF196931 | 0.035714 | 0.04225 | 0.003706 | −0.3226 | ZFP95 |
| AJ223333.1 | 0.035714 | 0.035247 | 0.023593 | −0.32279 | DNMT2 |
| NM_005213.1 | 0.035714 | 0.04225 | 0.216305 | −0.32285 | CSTA |
| AF142419.1 | 0.035714 | 0.04225 | 0.013772 | −0.33425 | QKI |
| NM_020375.1 | 0.035714 | 0.035247 | 0.07666 | −0.33473 | C12orf5 |
| NM_021970.1 | 0.035714 | 0.04225 | 0.054987 | −0.33505 | MAP2K1IP1 |
| AK023816.1 | 0.035714 | 0.035247 | 0.136952 | −0.34214 | |
| NM_012238.3 | 0.035714 | 0.00747 | 0.018308 | −0.3438 | SIRT1 |
| AF205218.1 | 0.035714 | 0.035247 | 0.033088 | −0.34674 | NS1-BP |
| NM_001660.2 | 0.035714 | 0.035247 | 0.020387 | −0.35015 | ARF4 |
| NM_001196.1 | 0.035714 | 0.015406 | 0.020544 | −0.35511 | BID |
| NM_002970.1 | 0.035714 | 0.035247 | 0.064201 | −0.35676 | SAT |
| AC074331 | 0.035714 | 0.035247 | 0.01515 | −0.35767 | |
| M75715.1 | 0.035714 | 0.035247 | 0.010802 | −0.3577 | ETF1 |
| NM_018657.2 | 0.035714 | 0.04225 | 0.021807 | −0.3681 | MYNN |
| NM_003370.1 | 0.035714 | 0.035247 | 0.056143 | −0.37617 | VASP |
| AI761561 | 0.035714 | 0.035247 | 0.084393 | −0.37861 | HK2 |
| NM_002657.2 | 0.035714 | 0.035247 | 0.043402 | −0.39004 | PLAGL2 |
| NM_004565.1 | 0.035714 | 0.035247 | 0.086212 | −0.39025 | PEX14 |
| AK023837.1 | 0.035714 | 0.04225 | 0.083366 | −0.39056 | KIAA1025 |
| AL117354 | 0.035714 | 0.00747 | 0.011916 | −0.39754 | LOC50999 |
| NM_001356.2 | 0.035714 | 0.035247 | 0.029134 | −0.41337 | DDX3 |
| NM_018573.1 | 0.035714 | 0.015406 | 0.00529 | −0.41757 | PRO1068 |
| NM_030799.1 | 0.035714 | 0.035247 | 0.075235 | −0.42088 | SMAP-5 |
| AA524053 | 0.035714 | 0.04225 | 0.031361 | −0.42442 | |
| NM_002748.1 | 0.035714 | 0.035247 | 0.023266 | −0.42563 | MAPK6 |
| NM_002053.1 | 0.035714 | 0.035247 | 0.053201 | −0.43747 | GBP1 |
| AB023227.1 | 0.035714 | 0.04225 | 0.003343 | −0.43985 | KIAA1010 |
| AW193511 | 0.035714 | 0.035247 | 0.012709 | −0.44652 | HIS1 |
| AW272611 | 0.035714 | 0.04225 | 0.024277 | −0.44899 | TMPO |
| AI671747 | 0.035714 | 0.00747 | 0.02288 | −0.45263 | MISS |
| AI688580 | 0.035714 | 0.035247 | 0.035918 | −0.45484 | SURB7 |
| NM_002502.1 | 0.035714 | 0.035247 | 0.107712 | −0.45745 | NFKB2 |
| NM_004267.1 | 0.035714 | 0.04225 | 0.030352 | −0.47177 | CHST2 |
| X15132.1 | 0.035714 | 0.035247 | 0.041452 | −0.47259 | SOD2 |
| NM_012093.1 | 0.035714 | 0.035247 | 0.011606 | −0.47474 | AK5 |
| D26067.1 | 0.035714 | 0.035247 | 0.003438 | −0.47644 | KIAA0033 |
| NM_001166.2 | 0.035714 | 0.04225 | 0.015252 | −0.48334 | BIRC2 |
| NM_016545.1 | 0.035714 | 0.035247 | 0.029826 | −0.48723 | IER5 |
| NM_021122.2 | 0.035714 | 0.035247 | 0.070882 | −0.49855 | FACL2 |
| NM_017936.1 | 0.035714 | 0.035247 | 0.008293 | −0.5016 | FLJ20707 |
| NM_000574.1 | 0.035714 | 0.035247 | 0.022743 | −0.50532 | DAF |
| AL050144.1 | 0.035714 | 0.015406 | 0.000822 | −0.52839 | ZNF363 |
| NM_005346.2 | 0.035714 | 0.035247 | 0.10824 | −0.5359 | HSPA1B |
| NM_022725.1 | 0.035714 | 0.035247 | 0.023814 | −0.53779 | FANCF |
| AI348010 | 0.035714 | 0.035247 | 0.226116 | −0.54348 | |
| AI927993 | 0.035714 | 0.035247 | 0.054067 | −0.54478 | OSBP |
| BE327172 | 0.035714 | 0.035247 | 0.091317 | −0.54925 | JUN |
| AI741876 | 0.035714 | 0.00747 | 0.025182 | −0.57505 | |
| NM_003107.1 | 0.035714 | 0.035247 | 0.078087 | −0.59709 | SOX4 |
| BE383139 | 0.035714 | 0.035247 | 0.00951 | −0.60058 | RARA |
| NM_018398.1 | 0.035714 | 0.035247 | 0.016221 | −0.603 | CACNA2D3 |
| NM_000201.1 | 0.035714 | 0.035247 | 0.036915 | −0.62554 | ICAM1 |
| NM_002229.1 | 0.035714 | 0.035247 | 0.129802 | −0.64436 | JUNB |
| NM_021960.1 | 0.035714 | 0.04225 | 0.024176 | −0.66914 | MCL1 |
| NM_016010.1 | 0.035714 | 0.00747 | 0.015684 | −0.68307 | LOC51101 |
| NM_004417.2 | 0.035714 | 0.035247 | 0.043377 | −0.68458 | DUSP1 |
| NM_025195.1 | 0.035714 | 0.035247 | 0.055882 | −0.68638 | C8FW |
| NM_004418.2 | 0.035714 | 0.035247 | 0.306591 | −0.68934 | DUSP2 |
| AB017493.1 | 0.035714 | 0.015406 | 0.010224 | −0.6982 | COPEB |
| AF064824.1 | 0.035714 | 0.035247 | 0.010071 | −0.70109 | RIPK2 |
| NM_005354.2 | 0.035714 | 0.035247 | 0.04394 | −0.70667 | JUND |
| NM_006469.1 | 0.035714 | 0.035247 | 0.006453 | −0.71493 | NS1-BP |
| NM_006290.1 | 0.035714 | 0.035247 | 0.155375 | −0.73437 | TNFAIP3 |
| AI339541 | 0.035714 | 0.035247 | 0.039838 | −0.76402 | JUND |
| AF087853.1 | 0.035714 | 0.035247 | 0.076647 | −0.77217 | GADD45B |
| AL031602 | 0.035714 | 0.035247 | 0.015158 | −0.78504 | |
| BF575213 | 0.035714 | 0.035247 | 0.007144 | −0.78651 | |
| M68956.1 | 0.035714 | 0.00747 | 0.001933 | −0.79718 | MARCKS |
| NM_004907.1 | 0.035714 | 0.035247 | 0.006503 | −0.81053 | ETR101 |
| AW083357 | 0.035714 | 0.035247 | 0.009893 | −0.81405 | IL1RN |
| AF153820.1 | 0.035714 | 0.035247 | 0.004403 | −0.82757 | KCNJ2 |
| AI608725 | 0.035714 | 0.035247 | 0.010119 | −0.83319 | ICAM1 |
| NM_000958.1 | 0.035714 | 0.015406 | 0.003112 | −0.86354 | PTGER4 |
| AA083483 | 0.035714 | 0.035247 | 0.012228 | −0.88452 | FTH1 |
| NM_002664.1 | 0.035714 | 0.00747 | 0.000855 | −0.88664 | PLEK |
| AL031602 | 0.035714 | 0.015406 | 0.00038 | −0.88673 | |
| W27419 | 0.035714 | 0.015406 | 0.000361 | −0.95575 | |
| NM_002852.1 | 0.035714 | 0.035247 | 0.001716 | −0.97365 | PTX3 |
| NM_001964.1 | 0.035714 | 0.035247 | 0.068287 | −0.99045 | EGR1 |
| AF078077.1 | 0.035714 | 0.035247 | 0.017058 | −1.003 | GADD45B |
| NM_015714.1 | 0.035714 | 0.035247 | 0.210858 | −1.05996 | G0S2 |
| BC004490.1 | 0.035714 | 0.035247 | 0.068201 | −1.06388 | FOS |
| AI738896 | 0.035714 | 0.035247 | 0.071526 | −1.09453 | TNFAIP3 |
| AW973834 | 0.035714 | 0.035247 | 0.030817 | −1.09468 | |
| NM_004895.1 | 0.035714 | 0.035247 | 0.004992 | −1.10724 | CIAS1 |
| U08839.1 | 0.035714 | 0.035247 | 0.030968 | −1.1245 | PLAUR |
| BC005020.1 | 0.035714 | 0.035247 | 0.022893 | −1.13801 | PPIF |
| NM_005627.1 | 0.035714 | 0.035247 | 0.010834 | −1.16132 | SGK |
| NM_015675.1 | 0.035714 | 0.035247 | 0.022251 | −1.16822 | GADD45B |
| AI433595 | 0.035714 | 0.015406 | 0.002395 | −1.17663 | PLEK |
| NM_002135.1 | 0.035714 | 0.035247 | 0.009942 | −1.19934 | NR4A1 |
| NM_003407.1 | 0.035714 | 0.035247 | 0.0028 | −1.30448 | ZFP36 |
| NM_004233.1 | 0.035714 | 0.035247 | 0.045128 | −1.33091 | CD83 |
| NM_001432.1 | 0.035714 | 0.035247 | 0.001942 | −1.33633 | EREG |
| NM_002228.2 | 0.035714 | 0.035247 | 0.007227 | −1.34352 | JUN |
| NM_004049.1 | 0.035714 | 0.035247 | 0.004927 | −1.41895 | BCL2A1 |
| U83981 | 0.035714 | 0.035247 | 0.005806 | −1.46885 | PPP1R15A |
| NM_006018.1 | 0.035714 | 0.035247 | 0.002094 | −1.50671 | HM74 |
| BG491844 | 0.035714 | 0.035247 | 0.011957 | −1.61438 | JUN |
| BC002646.1 | 0.035714 | 0.035247 | 0.002438 | −1.64136 | JUN |
| NM_000963.1 | 0.035714 | 0.035247 | 0.025772 | −1.65759 | PTGS2 |
| AY029180.1 | 0.035714 | 0.035247 | 0.011082 | −1.69399 | PLAUR |
| NM_014330.2 | 0.035714 | 0.035247 | 0.003245 | −1.74665 | PPP1R15A |
| NM_003897.1 | 0.035714 | 0.035247 | 0.002981 | −1.89968 | IER3 |
| M57731.1 | 0.035714 | 0.035247 | 0.001811 | −1.9703 | CXCL2 |
| NM_000584.1 | 0.035714 | 0.035247 | 0.030747 | −2.54298 | IL8 |
| NM_000576.1 | 0.035714 | 0.035247 | 0.000992 | −2.66025 | IL1B |
| M15330 | 0.035714 | 0.035247 | 0.001505 | −2.71142 | IL1B |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identifies by a GenBank accession number mentioned in this specification are herein incorporated in their entirety by reference into the specification, to the same extent as if each individual publication, patent, patent application or sequence was specifically and individually indicated to be incorporated herein by reference. In addition, citation or identification of any reference in this application shall not be construed as an admission that such reference is available as prior art to the present invention.

1. A method of diagnosing a subject with multiple sclerosis, the method comprising determining a level of expression of at least one gene selected from the group consisting of the genes listed in Tables I-V in a sample obtained from the subject, wherein a substantial difference between said level of expression of said gene in said sample obtained from said subject and a normal expression level of said gene is an indication that the subject is afflicted with multiple sclerosis.

2. The method of claim 1, wherein said normal expression level of said at least one gene is determined by measuring said level of expression of said gene in at least one control sample obtained from at least one healthy individual.

3. The method of claim 2, wherein said sample includes peripheral blood mononuclear cells.

4. The method of claim 1, wherein said substantial difference is a difference statistically significant at a confidence level of p=0.05 as determined by at least one test selected from the group consisting of a t-test, a TNoM and an INFO score.

5. The method of claim 1, wherein said level of expression of said at least one gene is determined by quantifying a level of a protein product thereof in said sample.

6. The method of claim 5, wherein quantifying a level of said protein is effected using a reagent which specifically binds with said protein.

7. The method of claim 6, wherein said reagent comprises an antibody or fragments thereof.

8. The method of claim 1, wherein said at least one gene is selected from the genes listed in Table I.

9. The method of claim 1, wherein said at least one gene is selected from the genes listed in Table II.

10. The method of claim 1, wherein said at least one gene is selected from the genes listed in Table III.

11. The method of claim 1, wherein said at least one gene is selected from the genes listed in Table IV.

12. The method of claim 1, wherein said at least one gene is selected from the genes listed in Table V.

13. The method of claim 1, wherein the level of expression of said at least one gene in said sample is determined by detecting the presence in said sample of a transcribed polynucleotide or portion thereof.

14. The method of claim 13, wherein said transcribed polynucleotide is mRNA.

15. The method of claim 13, wherein said transcribed polynucleotide or portion thereof is detected via a labeled probe which specifically hybridizes with said transcribed polynucleotide or portion thereof.

16. The method of claim 1, wherein said sample from a subject is T cells, and said at least one gene is selected from the genes listed in Table IV and whereas said normal expression of said gene is T-cell expression.

17. The method of claim 16, wherein said substantial difference is at least a 1.5 fold change.

18. The method of claim 1, wherein said at least one gene comprises at least 10 genes each independently selected from the group consisting of the genes listed in Tables I-V.

19. The method of claim 1, wherein said at least one gene comprises at least 50 genes each independently selected from the group consisting of the genes listed in Tables I-V.

20. The method of claim 1, wherein said at least one gene comprises at least 100 genes each independently selected from the group consisting of the genes listed in Tables I-V.

21. The method of claim 1, wherein said at least one gene comprises at least 250 genes each independently selected from the group consisting of the genes listed in Tables I-V.

22. The method of claim 1, wherein said at least one gene comprises at least 500 genes each independently selected from the group consisting of the genes listed in Tables I-V.

23. The method of claim 1, wherein said at least one gene comprises at least 750 genes each independently selected from the group consisting of the genes listed in Tables I-V.

24. The method of claim 1, wherein said at least one gene comprises at least 1000 genes each independently selected from the group consisting of the genes listed in Tables I-V.

25. The method of claim 1, wherein said at least one gene comprises at least 1200 genes each independently selected from the group consisting of the genes listed in Tables I-V.

26. A method of diagnosing a subject with multiple sclerosis, the method comprising the step of determining a level of expression of each of the genes listed in Tables I-V in a sample obtained from the subject, wherein a substantial difference between expression levels of said genes in said sample obtained from said subject and normal expression levels of said genes is an indication that the subject is afflicted with multiple sclerosis.

27. The method of claim 26, wherein said normal expression levels of said genes is determined by measuring said level of expression of said genes in at least one control sample obtained from at least one healthy individual.

28. The method of claim 26, wherein said sample includes peripheral blood mononuclear cells.

29. The method of claim 26, wherein said substantial difference is a difference statistically significant at a confidence level of p=0.05 as determined by at least one test selected from the group consisting of a t-test, a TNoM and an INFO score.

30. The method of claim 26, wherein said level of expression of said genes is determined by quantifying a level of a protein product thereof in said sample.

31. The method of claim 30, wherein quantifying a level of said protein is effected using a reagent which specifically binds with said protein.

32. The method of claim 31, wherein said reagent comprises an antibody or fragments thereof.

33. The method of claim 26, wherein the level of expression of said genes in said sample is determined by detecting the presence in said sample of a transcribed polynucleotide or portion thereof.

34. The method of claim 33, wherein said transcribed polynucleotide is mRNA.

35. The method of claim 34, wherein said transcribed polynucleotide or portion thereof is detected via a labeled probe which specifically hybridizes with said transcribed polynucleotide or portion thereof.

36. A method of monitoring a state of multiple sclerosis in a subject, the method comprising monitoring a level of expression of at least one gene selected from the group consisting of the genes listed in Tables I-V over a predetermined time period, wherein substantial difference between the levels of expression of said at least one gene over said predetermined time period indicates a change in a state of the multiple sclerosis in the subject.

37. The method of claim 36, wherein monitoring said level of expression of at least one gene over said predetermined time period is effected by periodically obtaining a sample from the individual and determining said level of expression of said at least one gene in said sample.

38. The method of claim 37, wherein said sample includes peripheral blood mononuclear cells.

39. The method of claim 36, wherein said substantial difference is a difference statistically significant at a confidence level of p=0.05 as determined by at least one test selected from the group consisting of a t-test, a TNoM and an INFO score.

40. The method of claim 36, wherein said level of expression of said at least one gene is determined by quantifying a level of a protein product thereof in said sample.

41. The method of claim 36, wherein quantifying a level of said protein is effected using a reagent which specifically binds with said protein.

42. The method of claim 41, wherein said reagent comprises an antibody or fragments thereof.

43. The method of claim 36, wherein said at least one gene is selected from the genes listed in Table I.

44. The method of claim 36, wherein said at least one gene is selected from the genes listed in Table II.

45. The method of claim 36, wherein said at least one gene is selected from the genes listed in Table III.

46. The method of claim 36, wherein said at least one gene is selected from the genes listed in Table IV.

47. The method of claim 36, wherein said at least one gene is selected from the genes listed in Table V.

48. The method of claim 36, wherein the level of expression of said at least one gene in said sample is determined by detecting the presence in said sample of a transcribed polynucleotide or portion thereof.

49. The method of claim 48, wherein said transcribed polynucleotide is mRNA.

50. The method of claim 48, wherein said transcribed polynucleotide or portion thereof is detected via a labeled probe which specifically hybridizes with said transcribed polynucleotide or portion thereof.

51. The method of claim 36, wherein said sample from a subject is T cells, and said at least one gene is selected from the genes listed in Table IV and whereas said normal expression of said gene is T-cell expression.

52. The method of claim 51, wherein said substantial difference is at least a 1.5 fold change.

53. The method of claim 36, wherein said at least one gene comprises at least 10 genes each independently selected from the group consisting of the genes listed in Tables I-V.

54. The method of claim 36, wherein said at least one gene comprises at least 50 genes each independently selected from the group consisting of the genes listed in Tables I-V.

55. The method of claim 36, wherein said at least one gene comprises at least 100 genes each independently selected from the group consisting of the genes listed in Tables I-V.

56. The method of claim 36, wherein said at least one gene comprises at least 250 genes each independently selected from the group consisting of the genes listed in Tables I-V.

57. The method of claim 36, wherein said at least one gene comprises at least 500 genes each independently selected from the group consisting of the genes listed in Tables I-V.

58. The method of claim 36, wherein said at least one gene comprises at least 750 genes each independently selected from the group consisting of the genes listed in Tables I-V.

59. The method of claim 36, wherein said at least one gene comprises at least 1000 genes each independently selected from the group consisting of the genes listed in Tables I-V.

60. The method of claim 36, wherein said at least one gene comprises at least 1200 genes each independently selected from the group consisting of the genes listed in Tables I-V.

61. A method of assessing the efficacy of a treatment regimen on multiple sclerosis in a subject, the method comprising determining a level of expression of at least one gene selected from the group consisting of the genes listed in Tables I-V in samples obtained from the subject prior to, and following exposure to the treatment regimen, wherein a substantial difference in the expression level of said at least one gene between said samples is an indication that the treatment regimen is efficacious in treating multiple sclerosis in said subject.

62. The method of claim 61, wherein said treatment regimen is administering at least one test compound for inhibiting multiple sclerosis.

63. The method of claim 61, wherein said treatment regimen is an environmental condition.

64. The method of claim 61, wherein said samples include peripheral blood mononuclear cells.

65. The method of claim 61, wherein said substantial difference is a difference statistically significant at a confidence level of p=0.05 as determined by at least one test selected from the group consisting of a t-test, a TNoM and an INFO score.

66. The method of claim 61, wherein said level of expression of said at least one gene is determined by quantifying a level of a protein product thereof in said sample.

67. The method of claim 66, wherein quantifying a level of said protein is effected using a reagent which specifically binds with said protein.

68. The method of claim 67, wherein said reagent comprises an antibody or fragments thereof.

69. The method of claim 61, wherein said at least one gene is selected from the genes listed in Table I.

70. The method of claim 61, wherein said at least one gene is selected from the genes listed in Table II.

71. The method of claim 61, wherein said at least one gene is selected from the genes listed in Table III.

72. The method of claim 61, wherein said at least one gene is selected from the genes listed in Table IV.

73. The method of claim 61, wherein said at least one gene is selected from the genes listed in Table V.

74. The method of claim 61, wherein the level of expression of said at least one gene in said samples is determined by detecting the presence in said samples of a transcribed polynucleotide or portion thereof.

75. The method of claim 74, wherein said transcribed polynucleotide is mRNA.

76. The method of claim 74, wherein said transcribed polynucleotide or portion thereof is detected via a labeled probe which specifically hybridizes with said transcribed polynucleotide or portion thereof.

77. The method of claim 61, wherein said at least one gene comprises at least 10 genes each independently selected from the group consisting of the genes listed in Tables I-V.

78. The method of claim 61, wherein said at least one gene comprises at least 50 genes each independently selected from the group consisting of the genes listed in Tables I-V.

79. The method of claim 61, wherein said at least one gene comprises at least 100 genes each independently selected from the group consisting of the genes listed in Tables I-V.

80. The method of claim 61, wherein said at least one gene comprises at least 250 genes each independently selected from the group consisting of the genes listed in Tables I-V.

81. The method of claim 61, wherein said at least one gene comprises at least 500 genes each independently selected from the group consisting of the genes listed in Tables I-V.

82. The method of claim 61, wherein said at least one gene comprises at least 750 genes each independently selected from the group consisting of the genes listed in Tables I-V.

83. The method of claim 61, wherein said at least one gene comprises at least 1000 genes each independently selected from the group consisting of the genes listed in Tables I-V.

84. The method of claim 61, wherein said at least one gene comprises at least 1200 genes each independently selected from the group consisting of the genes listed in Tables I-V.

85. A kit for diagnosing multiple sclerosis in a subject, the kit comprising components suitable for determining expression levels of at least one gene selected from the group of genes listed in Tables I-V.

86. The kit of claim 85, wherein said reagents include at least one polynucleotide sequence selected capable of specifically hybridizing with an transcription product of said at least one gene and reagents for detecting and optionally quantifying a complex formed from said at least one polynucleotide sequence and said transcription product.

87. The kit of claim 85, wherein said reagents include at least one antibody selected capable of specifically binding a polypeptide product of said at least one gene and reagents for detecting and optionally quantifying a complex formed from said at least one antibody and said polypeptide product.

88. The kit of claim 85, wherein said at least one gene is selected from the genes listed in Table I.

89. The kit of claim 85, wherein said at least one gene is selected from the genes listed in Table II.

90. The kit of claim 85, wherein said at least one gene is selected from the genes listed in Table III.

91. The method of claim 88, wherein said at least one gene is selected from the genes listed in Table IV.

92. The method of claim 85, wherein said at least one gene is selected from the genes listed in Table V.

93. The kit of claim 85, wherein the kit further comprises packaging material identifying the kit as useful from diagnosing MS.

94. A polynucleotide array comprising at least 10 and no more than 1500 polynucleotide sequences, wherein each of said sequences is selected capable of hybridizing with a transcription product of a polynucleotide sequence of a gene selected from the group of genes listed in Tables I-V.

95. The polynucleotide array of claim 94, wherein said array is selected having polynucleotide sequences capable of diagnosing subjects suspected of suffering from multiple sclerosis.

96. The polynucleotide array of claim 94, wherein said array is selected having polynucleotide sequences capable of diagnosing subjects suspected of suffering from probable multiple sclerosis.

97. The polynucleotide array of claim 94, wherein said array is selected capable of diagnosing subjects suspected of suffering from primary progressive multiple sclerosis.

98. The polynucleotide array of claim 94, wherein said array is selected capable of diagnosing subjects suspected of suffering from relapsing multiple sclerosis.

99. The polynucleotide array of claim 94, wherein said gene is selected from the genes listed in Table I.

100. The polynucleotide array of claim 94, wherein said gene is selected from the genes listed in Table II.

101. The polynucleotide array of claim 94, wherein said gene is selected from the genes listed in Table III.

102. The polynucleotide array of claim 94, wherein said gene is selected from the genes listed in Table IV.

103. The polynucleotide array of claim 94, wherein said gene is selected from the genes listed in Table V.

104. An array comprising at least 10 and no more than 1500 antibodies or antibody fragments each capable of specifically binding a protein product of a gene selected from the group of genes listed in Tables I-V.

105. The array of claim 104, wherein said array is selected having antibodies or antibody fragments capable of diagnosing subjects suspected of suffering from multiple sclerosis.

106. The array of claim 104, wherein said array is selected having antibodies or antibody fragments capable of diagnosing subjects suspected of suffering from probable multiple sclerosis.

107. The array of claim 104, wherein said array is selected capable of diagnosing subjects suspected of suffering from primary progressive multiple sclerosis.

108. The array of claim 104, wherein said array is selected capable of diagnosing subjects suspected of suffering from relapsing multiple sclerosis.

109. The array of claim 104, wherein said gene is selected from the genes listed in Table I.

110. The array of claim 104, wherein said gene is selected from the genes listed in Table II.

111. The array of claim 104, wherein said gene is selected from the genes listed in Table III.

112. The array of claim 104, wherein said gene is selected from the genes listed in Table IV.

113. The array of claim 104, wherein said gene is selected from the genes listed in Table V.
